(19) United States
(12) Patent Application Publication
(10) Pub. No.: US 2025/0134573 A1
(43) Pub. Date: May 1, 2025

(54) MULTI-APPLICATOR SYSTEM AND METHOD FOR BODY CONTOURING

(71) Applicant: Zeltiq Aesthetics, Inc., Pleasanton, CA (US)

(72) Inventors: Mark William Baker, Livermore, CA (US); Joseph Coakley, Dublin, CA (US); George Frangineas, JR., Fremont, CA (US); Russell Lee Gauthier, Pleasanton, CA (US); Roy Christopher Gomes, San Ramon, CA (US); Clive Heke, Pleasanton, CA (US); Kurt Arthur Kordes, Livermore, CA (US); Mark Allen McCall, Berkeley, CA (US); William P. Pennybacker, Livermore, CA (US); Austin Root, Pleasanton, CA (US); Ralph Edward Saunders, San Ramon, CA (US); Teri L. Stivers, Livermore, CA (US); Peter Yee, San Ramon, CA (US); Tamara Hilton, Prescott, AZ (US)

(21) Appl. No.: 18/787,749

(22) Filed: Jul. 29, 2024

Related U.S. Application Data
(63) Continuation of application No. 17/402,354, filed on Aug. 13, 2021, now abandoned.
(60) Provisional application No. 63/065,946, filed on Aug. 14, 2020.

(51) Int. Cl. A61B 18/02; A61B 18/00

(52) U.S. Cl. CPC A61B 18/02; A61B 2018/00464; A61B 2018/00714; A61B 2018/00738; A61B 2018/00964; A61B 2018/0256

(57) ABSTRACT

Systems, methods, and devices for treating a subject are described herein. In some embodiments, an applicator for selectively affecting a subject's subcutaneous tissue is provided. The applicator can include: a housing; a treatment cup mounted in the housing, wherein the treatment cup defines a tissue-receiving cavity and includes a temperature-controlled surface; at least one thermal device coupled to the treatment cup and configured to receive energy via a flexible connector coupled to the applicator and to cool the temperature-controlled surface; an at least one vacuum port coupled to the treatment cup and configured to provide a vacuum to draw the subject's tissue into the tissue-receiving cavity and against at least a portion of a treatment area of the temperature-controlled surface to selectively damage and/or reduce the subject's subcutaneous tissue.

### CROSS-REFERENCE TO RELATED APPLICATION

This application claims priority to and the benefit of U.S. Provisional Patent Application No. 63/065,946, filed Aug. 14, 2020, entitled “MULTI-APPLICATOR SYSTEM AND METHOD FOR BODY CONTOURING,” which is incorporated herein by reference in its entirety.

### INCORPORATION BY REFERENCE OF APPLICATIONS AND PATENTS

The following commonly assigned U.S. Patent Applications and U.S. Patents are incorporated herein by reference in their entireties:
U.S. Patent Publication No. 2008/0287839 entitled “METHOD OF ENHANCED REMOVAL OF HEAT FROM SUBCUTANEOUS LIPID-RICH CELLS AND TREATMENT APPARATUS HAVING AN ACTUATOR”;
U.S. Pat. No. 6,032,6175 entitled “FREEZING METHOD FOR CONTROLLED REMOVAL OF FATTY TISSUE BY LIPOSUCTION”;
U.S. Patent Publication No. 2007/0255362 entitled “CRYOPROTECTANT FOR USE WITH A TREATMENT DEVICE FOR IMPROVED COOLING OF SUBCUTANEOUS LIPID-RICH CELLS”;
U.S. Pat. No. 7,854,754 entitled “COOLING DEVICE FOR REMOVING HEAT FROM SUBCUTANEOUS LIPID-RICH CELLS”;
U.S. Pat. No. 8,337,539 entitled “COOLING DEVICE FOR REMOVING HEAT FROM SUBCUTANEOUS LIPID-RICH CELLS”;
U.S. Patent Publication No. 2008/0077201 entitled “COOLING DEVICES WITH FLEXIBLE SENSORS”;
U.S. Pat. No. 9,132,031 entitled “COOLING DEVICE HAVING A PLURALITY OF CONTROLLABLE COOLING ELEMENTS TO PROVIDE A PREDETERMINED COOLING PROFILE”;
U.S. Patent Publication No. 2009/0118722, filed Oct. 31, 2007, entitled “METHOD AND APPARATUS FOR COOLING SUBCUTANEOUS LIPID-RICH CELLS OR TISSUE”;
U.S. Patent Publication No. 2009/0018624 entitled “LIMITING USE OF DISPOSABLE SYSTEM PATIENT PROTECTION DEVICES”;
U.S. Pat. No. 8,523,927 entitled “SYSTEM FOR TREATING LIPID-RICH REGIONS”;
U.S. Patent Publication No. 2009/0018625 entitled “MANAGING SYSTEM TEMPERATURE TO REMOVE HEAT FROM LIPID-RICH REGIONS”;
U.S. Patent Publication No. 2009/0018627 entitled “SECURE SYSTEM FOR REMOVING HEAT FROM LIPID-RICH REGIONS”;
U.S. Patent Publication No. 2009/0018626 entitled “USER INTERFACES FOR A SYSTEM THAT REMOVES HEAT FROM LIPID-RICH REGIONS”;
U.S. Pat. No. 6,041,787 entitled “USE OF CRYOPROTECTIVE AGENT COMPOUNDS DURING CRYOSURGERY”;
U.S. Pat. No. 8,285,390 entitled “MONITORING THE COOLING OF SUBCUTANEOUS LIPID-RICH CELLS, SUCH AS THE COOLING OF ADIPOSE TISSUE”;
U.S. Pat. No. 8,275,442 entitled “TREATMENT PLANNING SYSTEMS AND METHODS FOR BODY CONTOURING APPLICATIONS”;
U.S. patent application Ser. No. 12/275,002 entitled “APPARATUS WITH HYDROPHILIC RESERVOIRS FOR COOLING SUBCUTANEOUS LIPID-RICH CELLS”;
U.S. patent application Ser. No. 12/275,014 entitled “APPARATUS WITH HYDROPHOBIC FILTERS FOR REMOVING HEAT FROM SUBCUTANEOUS LIPID-RICH CELLS”;
U.S. Pat. No. 8,603,073 entitled “SYSTEMS AND METHODS WITH INTERRUPT/RESUME CAPABILITIES FOR COOLING SUBCUTANEOUS LIPID-RICH CELLS”;
U.S. Pat. No. 8,192,474 entitled “TISSUE TREATMENT METHODS”;
U.S. Pat. No. 8,702,774 entitled “DEVICE, SYSTEM AND METHOD FOR REMOVING HEAT FROM SUBCUTANEOUS LIPID-RICH CELLS”;
U.S. Pat. No. 8,676,338 entitled “COMBINED MODALITY TREATMENT SYSTEMS, METHODS AND APPARATUS FOR BODY CONTOURING APPLICATIONS”;
U.S. Pat. No. 9,314,368 entitled “HOME-USE APPLICATORS FOR NON-INVASIVELY REMOVING HEAT FROM SUBCUTANEOUS LIPID-RICH CELLS VIA PHASE CHANGE COOLANTS, AND ASSOCIATED DEVICES, SYSTEMS AND METHODS”;
U.S. Publication No. 2011/0238051 entitled “HOME-USE APPLICATORS FOR NON-INVASIVELY REMOVING HEAT FROM SUBCUTANEOUS LIPID-RICH CELLS VIA PHASE CHANGE COOLANTS, AND ASSOCIATED DEVICES, SYSTEMS AND METHODS”;
U.S. Publication No. 2012/02317023 entitled “DEVICES, APPLICATION SYSTEMS AND METHODS WITH LOCALIZED HEAT FLUX ZONES FOR REMOVING HEAT FROM SUBCUTANEOUS LIPID-RICH CELLS”;
U.S. Pat. No. 9,545,523 entitled “MULTI-MODALITY TREATMENT SYSTEMS, METHODS AND APPARATUS FOR ALTERING SUBCUTANEOUS LIPID-RICH TISSUE”;
U.S. Patent Publication No. 2014/0277302 entitled “TREATMENT SYSTEMS WITH FLUID MIXING SYSTEMS AND FLUID-COOLED APPLICATORS AND METHODS OF USING THE SAME”;
U.S. Pat. No. 9,132,031 entitled “COOLING DEVICE HAVING A PLURALITY OF CONTROLLABLE COOLING ELEMENTS TO PROVIDE A PREDETERMINED COOLING PROFILE”;
U.S. Pat. No. 8,285,390 entitled “MONITORING THE COOLING OF SUBCUTANEOUS LIPID-RICH CELLS, SUCH AS THE COOLING OF ADIPOSE TISSUE”;
U.S. Patent Publication No. 2016/0054101 entitled “TREATMENT SYSTEMS, SMALL VOLUME APPLICATORS, AND METHODS FOR TREATING SUBMENTAL TISSUE”;
U.S. Patent Publication No. 2018/0310950 entitled “SHALLOW SURFACE CRYOTHERAPY APPLICATORS AND RELATED TECHNOLOGY”;
U.S. Patent Publication No. 2020/0038234 entitled “METHODS, DEVICES, AND SYSTEMS FOR IMPROVING SKIN CHARACTERISTICS”; and
U.S. patent application Ser. No. 16/557,814 entitled COMPOSITIONS, TREATMENT SYSTEMS, AND METHODS FOR FRACTIONALLY FREEZING TISSUE.”

### TECHNICAL FIELD

The present disclosure relates generally to cryotherapy treatment systems and applicators.

### BACKGROUND

Excess body fat, or adipose tissue, may be present at various locations of a subject's body and may detract from personal appearance. Aesthetic improvement of the human body often involves the selective removal of adipose tissue located at the abdomen, thighs, buttocks, knees, submental region, face, and arms, as well as other locations. Invasive procedures (e.g., liposuction), however, tend to be associated with relative high costs, long recovery times, and increased risk of complications. Injection of drugs for reducing adipose tissue can cause significant swelling, bruising, pain, numbness, and/or induration.

Conventional non-invasive treatments for reducing adipose tissue often include regular exercise, application of topical agents, use of weight-loss drugs, dieting, or a combination of these treatments. One drawback of these non-invasive treatments is that they may not be effective or even possible under certain circumstances. For example, when a person is physically injured or ill, regular exercise may not be an option. Topical agents and orally administered weight-loss drugs are not an option if, as another example, they cause an undesirable reaction, such as an allergic or negative reaction. Additionally, non-invasive treatments may be ineffective for selectively reducing specific regions of adiposity, such as localized adipose tissue along the hips, abdomen, thighs, or the like.

### BRIEF DESCRIPTION OF THE DRAWINGS

In the drawings, identical reference numbers identify similar elements or acts.

FIG. 1A is a partially schematic, isometric view of a treatment system for non-invasively affecting target regions of a subject in accordance with an embodiment of the technology.

FIG. 1B is a schematic cross-sectional view of an applicator taken along line 1B-1B of FIG. 1A.

FIG. 1C is a schematic cross-sectional view of a connector taken along line 1C-1C of FIG. 1A.

FIG. 2A is a schematic block diagram illustrating components of a treatment system configured in accordance with embodiments of the present technology.

FIG. 2B is a schematic diagram of a cooling system of the treatment system of FIG. 2A.

FIG. 2C is a schematic diagram of a vacuum system of the treatment system of FIG. 2A.

FIGS. 3A-3I illustrate a vacuum applicator configured in accordance with embodiments of the present technology.

FIGS. 4A-4C illustrate a vacuum applicator configured in accordance with embodiments of the present technology.

FIGS. 5A and 5B illustrate a vacuum applicator configured in accordance with embodiments of the present technology.

FIGS. 6A and 6B illustrate a vacuum applicator configured in accordance with embodiments of the present technology.

FIGS. 7A and 7B illustrate a vacuum applicator configured in accordance with embodiments of the present technology.

FIGS. 8A and 8B illustrate a vacuum applicator configured in accordance with embodiments of the present technology.

FIGS. 9A-9D illustrate an applicator with a gel trap configured in accordance with embodiments of the present technology.

FIGS. 9E-9G illustrate the gel trap of FIGS. 9A-9D in accordance with embodiments of the present technology.

FIGS. 10A-10I illustrate a non-vacuum applicator configured in accordance with embodiments of the present technology.

FIGS. 11A and 11B illustrate tiled thermal devices suitable for use with non-vacuum applicators in accordance with embodiments of the present technology.

FIGS. 12A-18B illustrate applicator templates that can be used to select an applicator in accordance with embodiments of the present technology.

FIGS. 19A-19K illustrate a connector and associated components configured in accordance with embodiments of the present technology.

FIGS. 20A and 20B illustrate a cleaning cap in accordance with embodiments of the present technology.

FIG. 20C is a cross-sectional view of the cleaning cap of FIGS. 20A and 20B coupled to an applicator in accordance with embodiments of the present technology.

FIGS. 21A and 21B illustrate an applicator and connector assembly in accordance with embodiments of the present technology.

FIGS. 22A-22C illustrate a control unit configured in accordance with embodiments of the present technology.

FIG. 23 is a flowchart of a method for treating a subject in accordance with embodiments of the present technology.

FIG. 24 is a schematic block diagram illustrating subcomponents of a controller in accordance with embodiments of the present technology.

### DETAILED DESCRIPTION

The present disclosure describes treatment systems, applicators, and methods for affecting targeted sites. Several embodiments are directed to treatment systems having one or more of the following features:
(a) a plurality of different applicators that can be rapidly connected and/or disconnected from the system, thus allowing the applicators to be exchanged with each other as appropriate to tailor the treatment to a particular patient and/or treatment region; individual applicators can have treatment surfaces shaped to provide better contact with the skin surface and improve patient comfort;
(b) a cooling unit configured to provide faster and more efficient cooling of the tissue via the applicator(s);
(c) one or more vacuum units configured to provide more rapid and responsive application of vacuum pressure via the applicator(s) with little or no overshoot and/or undershoot;
(d) a control unit housing the electronic components for controlling and monitoring the treatment procedure;
(e) a connector configured to releasably couple to the applicators and/or the control unit to allow for rapid and simple interchange of system components, and also to facilitate cleaning and storage; and/or
(f) additional components and accessories, such as removable gel traps, applicator templates, cleaning caps, cards with security features and/or treatment profile information, etc.

Several of the details set forth below are provided to describe the following examples and methods in a manner sufficient to enable a person skilled in the relevant art to practice, make, and use them. Several of the details and advantages described below, however, may not be necessary to practice certain examples and methods of the technology. Additionally, the technology may include other examples and methods that are within the scope of the technology but are not described in detail.

Some aspects of the technology are directed to an applicator for selectively affecting a subject's subcutaneous tissue. The applicator can include a housing and a treatment cup mounted in the housing. The treatment cup can define a tissue-receiving cavity and include a temperature-controlled surface. The applicator can also include at least one thermal device coupled to the treatment cup and configured to receive energy via a flexible connector coupled to the applicator and to cool the temperature-controlled surface. The applicator can further include at least one vacuum port coupled to the treatment cup and configured to provide a vacuum to draw the subject's tissue into the tissue-receiving cavity and against at least a portion of a treatment area of the temperature-controlled surface to selectively damage and/or reduce the subject's subcutaneous tissue. The applicator can have one or more of the following: (a) a ratio of the treatment area to weight greater than or equal to 5 square inches per lb, or (b) a ratio of the treatment area to tissue-draw depth greater than or equal to 8 inches.

In another aspect, the present technology includes an apparatus for treating a subject's tissue. The apparatus includes at least one heat-exchanger plate having a cooling surface and at least one thermal unit thermally contacting the at least one heat-exchanger plate. The apparatus also includes a thermal feathering feature extending along at least portion of a perimeter of the at least one heat-exchanger plate. The thermal feathering feature can be in thermal contact with the at least one thermal unit such that a peripheral cooling surface of the thermal feathering feature is warmer than the cooling surface so that the subject's tissue directly underlying the peripheral cooling surface is damaged or reduced but to a lesser extent than the subject's targeted tissue directly below and cooled by the cooling surface.

In a further aspect, the present technology includes a kit for treating a subject's tissue. The kit includes plurality of applicators, each applicator including a treatment cup defining a tissue-receiving cavity and having a temperature-controlled surface configured to cool and selectively reduce the subject's tissue. At least some of the applicators can have different dimensions to treat differently-sized treatment sites. The kit also includes a connector configured to operably couple a single applicator to a control unit of a treatment system. Each applicator can include an interconnect section configured to releasably couple the applicator to the connector.

In yet another aspect, the present technology includes a treatment system for cooling and selectively affecting a subject's tissue. The treatment system can include at least one applicator including a treatment cup configured to be in thermal communication with the subject's tissue, and a control unit operably coupled to the at least one applicator. The control unit can include a cooling unit configured to cool the treatment cup of the at least one applicator, and at least one vacuum unit configured to apply a vacuum unit to the subject's tissue via the treatment cup. The at least one vacuum unit can be configured to reach a target vacuum pressure with at least one of (a) an amount of overshoot that is no more than 10% of the target pressure or (b) an amount of undershoot that is no more than 10% of the target pressure.

In still another aspect, the present technology includes a gel trap for fluidically coupling a vacuum line to a tissue-receiving cavity of an applicator. The gel trap includes a container configured to capture gel, and at least one sealing member configured to sealingly engage the applicator to fluidically couple the vacuum line to a vacuum port of the applicator such that the container captures gel drawn out of the tissue-receiving cavity while allowing air flow between the tissue-receiving cavity and the vacuum line to hold a subject's tissue in the tissue-receiving cavity.

Some of the embodiments disclosed herein can be for cosmetically beneficial alterations of target regions. Some cosmetic procedures may be for the sole purpose of altering a target region to conform to a cosmetically desirable look, feel, size, shape and/or other desirable cosmetic characteristic or feature. Accordingly, at least some embodiments of the cosmetic procedures can be performed without providing an appreciable therapeutic effect (e.g., no therapeutic effect). For example, some cosmetic procedures may not include restoration of health, physical integrity, or the physical well-being of a subject. The cosmetic methods can target subcutaneous regions to change a human subject's appearance and can include, for example, procedures performed on a subject's submental region, abdomen, hips, legs, arms, face, neck, ankle region, or the like. In other embodiments, however, cosmetically desirable treatments may have therapeutic outcomes (whether intended or not), such as psychological benefits, alteration of body hormone levels (by the reduction of adipose tissue), etc.

Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, stages, or characteristics may be combined in any suitable manner in one or more examples of the technology.

The headings provided herein are for convenience only and are not intended to limit or interpret the scope or meaning of the technology.

### A. Overview of the Technology

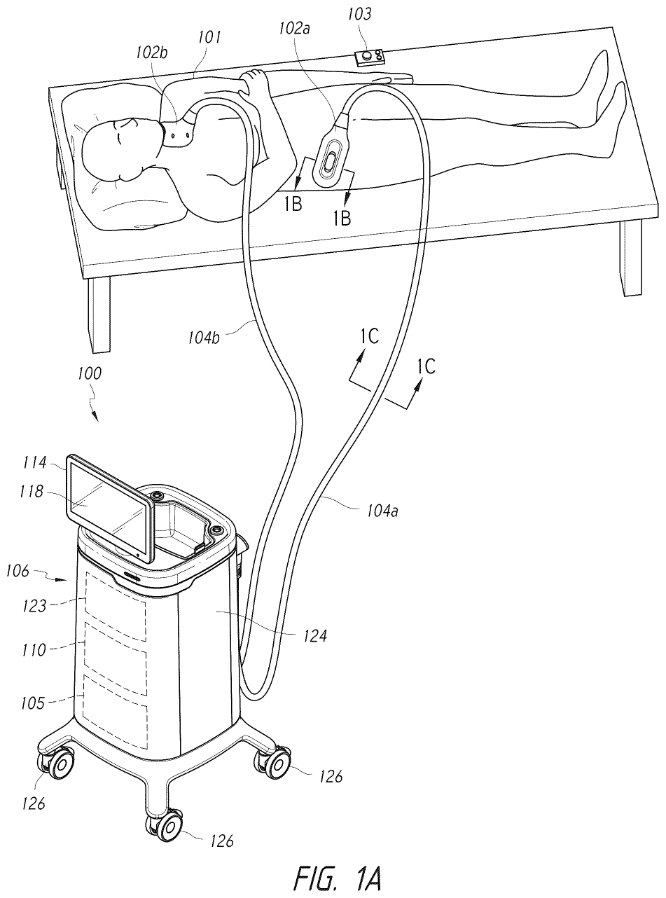

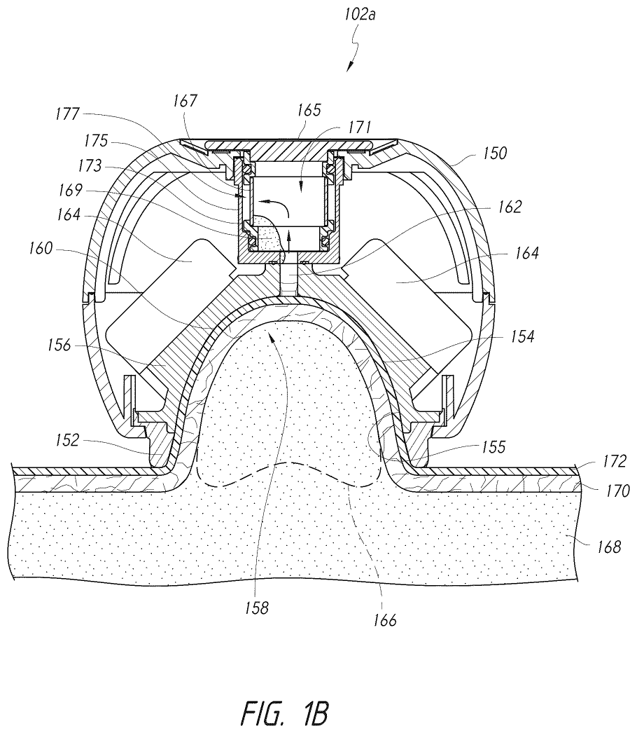

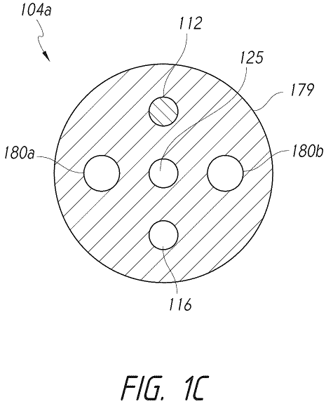

FIGS. 1A-1C and the following discussion provide a brief, general description of a treatment system 100 in accordance with some embodiments of the technology. Referring first to FIG. 1A, the treatment system 100 can be a temperature-controlled system for exchanging heat with a subject 101 and can include at least one non-invasive tissue-cooling apparatus in the form of a cooling cup applicator (“applicator”) configured to selectively cool tissue to affect targeted tissue, structures, or the like. In the illustrated embodiment, the treatment system 100 includes a first applicator 102a and a second applicator 102b (collectively, “applicators 102”). The first applicator 102a is positioned along the subject's hip and the second applicator 102b is positioned under the subject's chin. Each of the applicators 102 can draw a vacuum to provide suitable thermal contact with the subject's skin to cool subcutaneous adipose tissue. Each applicator 102 is configured to facilitate a high amount of thermal contact with the subject's skin by minimizing, limiting, or substantially eliminating air gaps at the applicator/tissue interface. The entire skin surface of the retained volume of tissue can be cooled for efficient treatment. Each applicator 102 can have a relatively shallow tissue-receiving chamber to avoid or limit pop offs (e.g., when an applicator pops off the subject due to a vacuum leak), air gaps, excess stretching of tissue, pooling of blood, rupturing of blood vessels, patient discomfort, and so forth.

The applicators 102 can be used to perform medical treatments to provide therapeutic effects and/or cosmetic procedures for cosmetically beneficial effects. Without being bound by theory, selective effects of cooling are believed to result in, for example, membrane disruption, cell shrinkage, disabling, disrupting, damaging, destroying, removing, killing, and/or other methods of lipid-rich cell alteration. Such alteration is believed to stem from one or more mechanisms acting alone or in combination. It is thought that such mechanism(s) trigger an apoptotic cascade, which is believed to be the dominant form of lipid-rich cell death by non-invasive cooling. In any of these embodiments, the effect of tissue cooling can be the selective reduction of lipid-rich cells by a desired mechanism of action, such as apoptosis, lipolysis, or the like. In some procedures, the applicators 102 can cool the skin surface and/or targeted tissue to cooling temperature in a range of from about −25° C. to about 20° C. In other embodiments, the cooling temperatures can be from about −20° C. to about 10° C., from about −18° C. to about 5° C., from about −15° C. to about 5° C., or from about −15° C. to about 0° C. In further embodiments, the cooling temperatures can be equal to or less than −5° C., −10° C., −15° C., or in yet another embodiment, from about −15° C. to about −25° C. Other cooling temperatures and temperature ranges can be used.

Apoptosis, also referred to as “programmed cell death”, is a genetically-induced death mechanism by which cells self-destruct without incurring damage to surrounding tissues. An ordered series of biochemical events induce cells to morphologically change. These changes include cellular blebbing, loss of cell membrane asymmetry and attachment, cell shrinkage, chromatin condensation and chromosomal DNA fragmentation. Injury via an external stimulus, such as cold exposure, is one mechanism that can induce cellular apoptosis in cells. Nagle, W. A., Soloff, B. L., Moss, A. J. Jr., Henle, K. J. “Cultured Chinese Hamster Cells Undergo Apoptosis After Exposure to Cold but Nonfreezing Temperatures” Cryobiology 27, 439-451 (1990).

One aspect of apoptosis, in contrast to cellular necrosis (a traumatic form of cell death causing local inflammation), is that apoptotic cells express and display phagocytic markers on the surface of the cell membrane, thus marking the cells for phagocytosis by macrophages. As a result, phagocytes can engulf and remove the dying cells (e.g., the lipid-rich cells) without eliciting an immune response. Temperatures that elicit these apoptotic events in lipid-rich cells may contribute to long-lasting and/or permanent reduction and reshaping of subcutaneous adipose tissue.

One mechanism of apoptotic lipid-rich cell death by cooling is believed to involve localized crystallization of lipids within the adipocytes at temperatures that do not induce crystallization in non-lipid-rich cells. The crystallized lipids may selectively injure these cells, inducing apoptosis (and may also induce necrotic death if the crystallized lipids damage or rupture the bi-lipid membrane of the adipocyte). Another mechanism of injury involves the lipid phase transition of those lipids within the cell's bi-lipid membrane, which results in membrane disruption or dysfunction, thereby inducing apoptosis. This mechanism is well-documented for many cell types and may be active when adipocytes, or lipid-rich cells, are cooled. Mazur, P., “Cryobiology: the Freezing of Biological Systems” Science, 68:939-949 (1970); Quinn, P. J., “A Lipid Phase Separation Model of Low Temperature Damage to Biological Membranes” Cryobiology, 22:128-147 (1985); Rubinsky, B., “Principles of Low Temperature Preservation” Heart Failure Reviews, 8, 277-284 (2003). Other possible mechanisms of adipocyte damage, described in U.S. Pat. No. 8,192,474, relate to ischemia/reperfusion injury that may occur under certain conditions when such cells are cooled as described herein. For instance, during treatment by cooling as described herein, the targeted adipose tissue may experience a restriction in blood supply and thus be starved of oxygen due to isolation as a result of applied pressure, cooling which may affect vasoconstriction in the cooled tissue, or the like. In addition to the ischemic damage caused by oxygen starvation and the buildup of metabolic waste products in the tissue during the period of restricted blood flow, restoration of blood flow after cooling treatment may additionally produce reperfusion injury to the adipocytes due to inflammation and oxidative damage that is known to occur when oxygenated blood is restored to tissue that has undergone a period of ischemia. This type of injury may be accelerated by exposing the adipocytes to an energy source (via, e.g., thermal, electrical, chemical, mechanical, acoustic, or other means) or otherwise increasing the blood flow rate in connection with or after cooling treatment as described herein. Increasing vasoconstriction in such adipose tissue by, e.g., various mechanical means (e.g., application of pressure or massage), chemical means or certain cooling conditions, as well as the local introduction of oxygen radical-forming compounds to stimulate inflammation and/or leukocyte activity in adipose tissue may also contribute to accelerating injury to such cells. Other yet-to-be understood mechanisms of injury may exist.

In addition to the apoptotic mechanisms involved in lipid-rich cell death, local cold exposure is also believed to induce lipolysis (i.e., fat metabolism) of lipid-rich cells and has been shown to enhance existing lipolysis which serves to further increase the reduction in subcutaneous lipid-rich cells. Vallerand, A. L., Zamecnik. J., Jones, P. J. H., Jacobs, I. “Cold Stress Increases Lipolysis, FFA Ra and TG/FFA Cycling in Humans” Aviation, Space and Environmental Medicine 70, 42-50 (1999).

One expected advantage of the foregoing techniques is that the subcutaneous lipid-rich cells in the target region can be reduced generally without collateral damage to non-lipid-rich cells in the same region. In general, lipid-rich cells can be affected at low temperatures that do not affect non-lipid-rich cells. As a result, lipid-rich cells, such as those associated with highly localized adiposity (e.g., adiposity along the abdomen, submental adiposity, submandibular adiposity, facial adiposity, etc.), can be affected while non-lipid-rich cells (e.g., myocytes) in the same generally region are not damaged. The unaffected non-lipid-rich cells can be located underneath lipid-rich cells (e.g., cells deeper than a subcutaneous layer of fat), in the dermis, in the epidermis, and/or at other locations.

In some procedures, the treatment system 100 can remove heat from underlying tissue through the upper layers of tissue and create a thermal gradient with the coldest temperatures near the cooling surface, or surfaces, of the applicators 102 (i.e., the temperature of the upper layer(s) of the skin can be lower than that of the targeted underlying target cells). It may be challenging to reduce the temperature of the targeted cells low enough to be destructive to these target cells (e.g., induce apoptosis, cell death, etc.) while also maintaining the temperature of the upper and surface skin cells high enough so as to be protective (e.g., non-destructive). The temperature difference between these two thresholds can be small (e.g., approximately, 5° C. to about 20° C., less than 5° C., less than 10° C., less than 15° C., less than 20° C., etc.). Protection of the overlying cells (e.g., typically water-rich dermal and epidermal skin cells) from freeze damage during dermatological and related aesthetic procedures that involve sustained exposure to cold temperatures may include improving the freeze tolerance and/or freeze avoidance of these skin cells by using, for example, cryoprotectants for inhibiting or preventing such freeze damage.

If an inadvertent skin freeze occurs, tissue can be rapidly rewarmed as soon as practicable after a skin freeze event has occurred to limit, reduce, or prevent damage and adverse side effects associated with the skin freeze event. After skin freezing begins, tissue can be rapidly warmed as soon as possible to minimize or limit damage to tissue, such as the epidermis. In some procedures, skin tissue is partially or completely intentionally frozen for a predetermined period of time and then warmed. According to one embodiment, an applicator can warm shallow tissue using, for example, thermoelectric elements in the device. Thermoelectric elements can include Peltier devices capable of operating to establish a desired temperature (or temperature profile) along the surface. In other embodiments, the applicator outputs energy to warm tissue. For example, the applicator can have electrodes that output radiofrequency energy for warming tissue. In some procedures, the tissue can be warmed at a rate of about 1° C./s, 2° C./s, 2.5° C./s, 3° C./s, 5° C./s, or other rate selected to thaw frozen tissue after the tissue has been partially or completely frozen for about 10 seconds, 30 seconds, 1 minute, 2 minutes, 5 minutes, 10 minutes, or other suitable length of time. If the subject 101 experiences discomfort (e.g., discomfort associated with skin freezing, excessive tissue draw, etc.), the subject 101 can use a notifier device 103 to summon the operator, clinician, physician, etc. In some embodiments, when the subject 101 presses a button of the notifier device 103, a healthcare worker is notified via a mobile device, such as a pager, a smartphone, etc. The healthcare worker can evaluate the subject 101 during and after warming of tissue. The system 100 can also perform additional monitoring in response to notifications to identify and monitor adverse events. The notifier device 103 can also include buttons for two-way communication (e.g., two-way talking via a local network or a wide area network), indicating discomfort level, or the like.

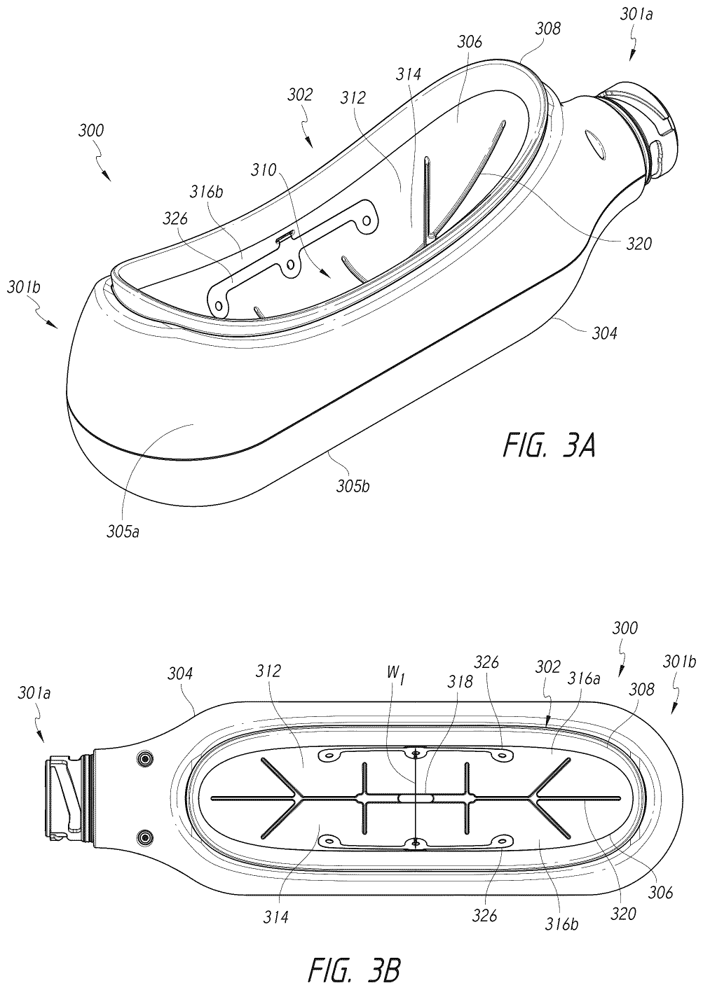

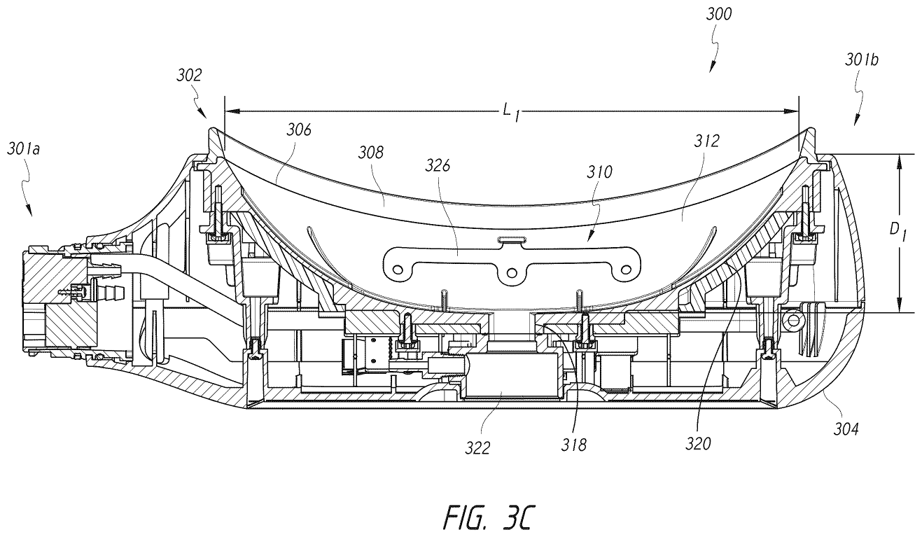

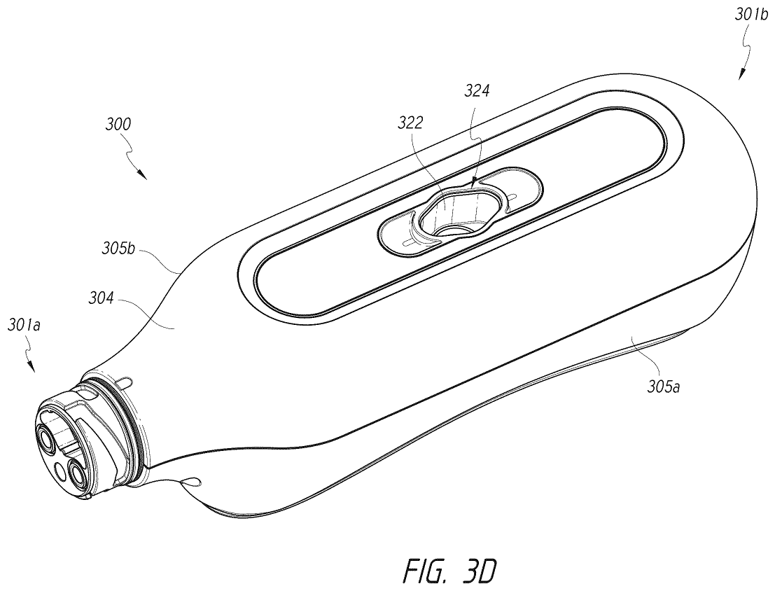

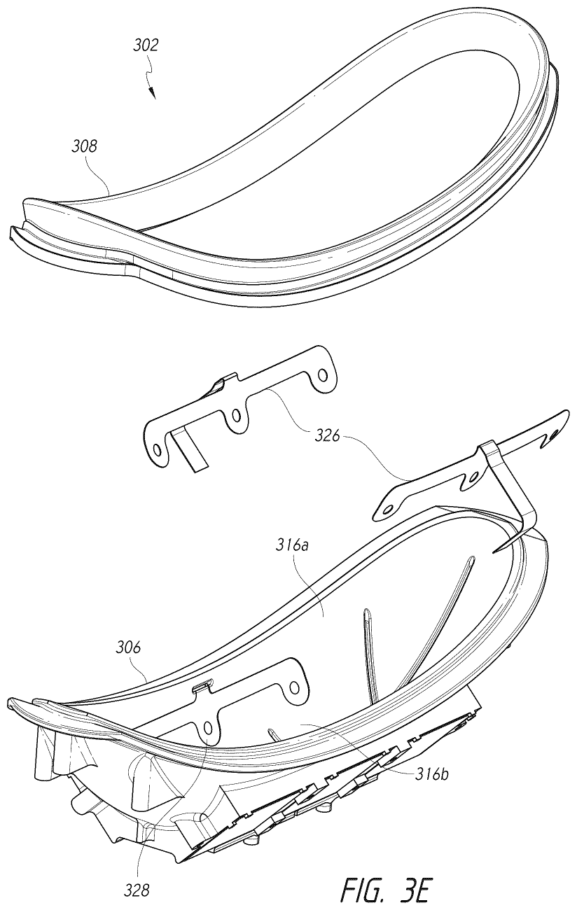

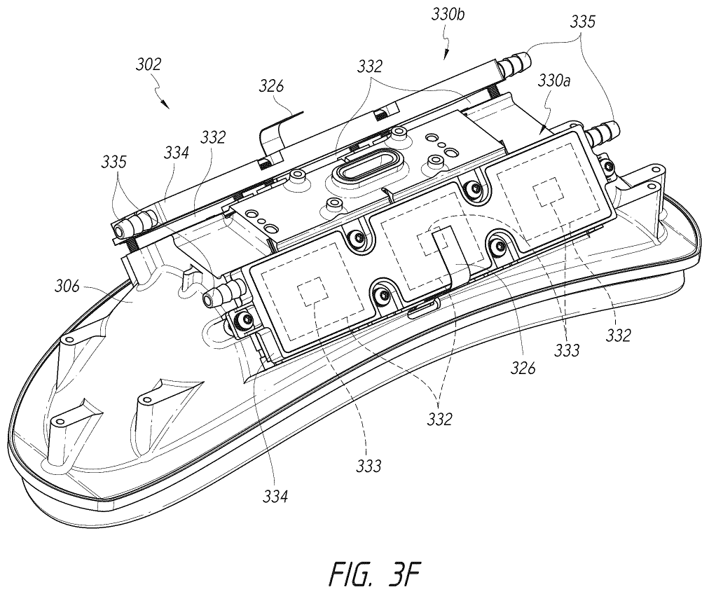

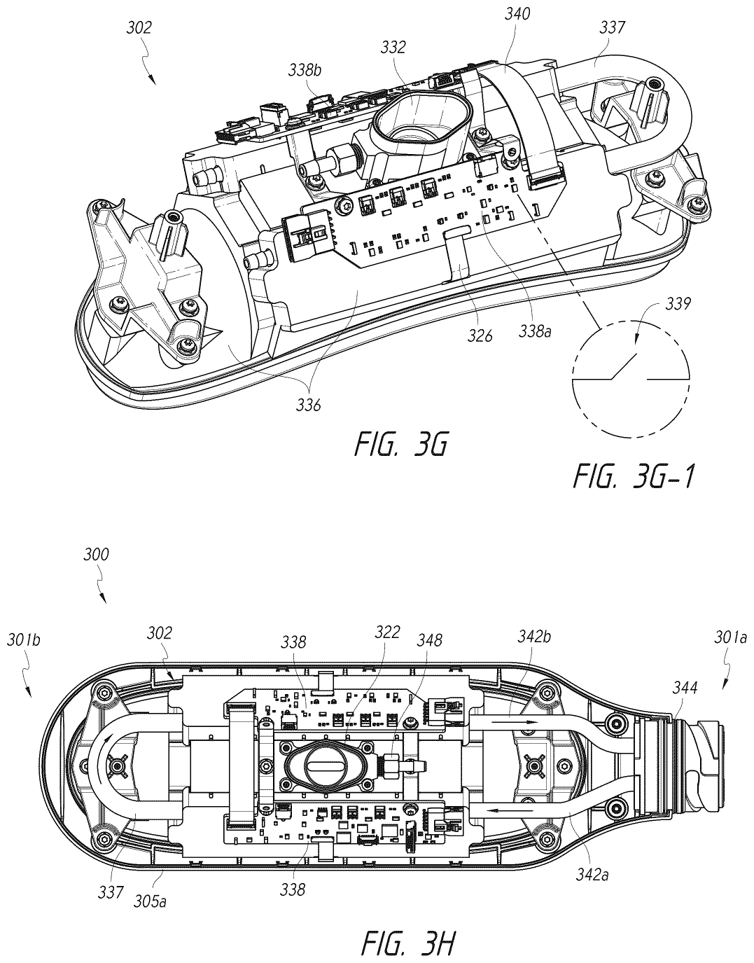

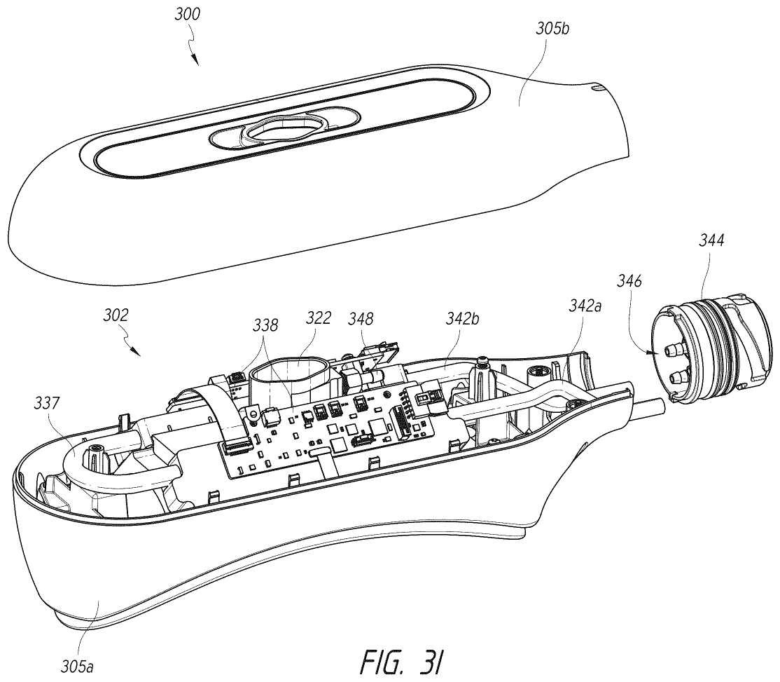

Although the illustrated applicators 102 of FIG. 1A are positioned along the hip and submental region, in other embodiments, the applicators 102 can also be positioned to treat tissue at the thighs, arms, buttock, abdomen, submandibular region, neck region, or other target regions. The applicators 102 can reduce localized adipose tissue along the abdomen, hips, submental region, or the like. It will be appreciated that the applicators 102 disclosed herein can be placed at other locations along the patient's body and the orientation of the applicator 102 can be selected to facilitate a relatively close fit. Additional examples of applicators are described in detail below in connection with FIGS. 3A-10I.

FIG. 1B is a schematic cross-sectional view of the first applicator 102a of FIG. 1A. The applicator 102a includes a housing 150 and a contoured lip or sealing element 152. The sealing element 152 can conform closely to contours of the subject's body to sealingly engage a skin surface 155. The housing 150 can support a cup 156 defining a tissue-receiving cavity 158 for holding tissue. The cup 156 can include a temperature-controlled surface 160 and a vacuum port 162. Suction can be applied to the patient's tissue via the vacuum port 162 to draw the skin surface 155 into contact with the temperature-controlled surface 160.

If a liner or gel pad (not shown) is used with the applicator 102a, the sealing element 152 can engage the liner or gel pad overlying the treatment site. For example, the liner can line the cup 156 and can be perforated such that a vacuum can be drawn through the liner to urge the subject's skin against the liner, thereby maintaining thermal contact between the tissue and the cup 156 via the liner. The cup 156 can be thermally conductive to efficiently cool the entire volume of targeted tissue retained in the applicator 102a.

The geometries of the cup 156 and sealing element 152 can be selected to conform to a contour of a cutaneous layer. For example, the shape of a typical human torso may vary between having a relatively large radius of curvature, e.g., on the stomach or back, and having a relatively small radius of curvature, e.g., on the abdominal sides. Accordingly, the tissue-receiving cavity 158 of the cup 156 can have a substantially U-shaped cross section, V-shaped cross section, or partially circular/elliptical cross-section, as well as or other cross-sectional shapes suitable for receiving tissue and matching body contours, and in particular shapes approximated by a higher-order parabolic polynomial (e.g., 4th order or higher). The thermal properties, shape, and/or configuration of the cup 156 can be selected based on, for example, target treatment temperatures and/or volume of the targeted tissue. The maximum depth of the tissue-receiving cavity 158 can be selected based on, for example, the volume of targeted tissue, characteristics of the targeted tissue, and/or desired level of patient comfort. Embodiments of the tissue-receiving cavity 158 for treating large volumes of tissue (e.g., adipose tissue along the abdomen, hips, buttock, etc.) can have a maximum depth equal to or less than about 2 cm, 5 cm, 10 cm, 15 cm, 20 cm, or 30 cm, for example. Embodiments of the tissue-receiving cavity 158 for treating small volumes (e.g., a small volume of submental tissue) can have a maximum depth equal to or less than about 0.5 cm, 2 cm, 2.5 cm, 3 cm, or 5 cm, for example. The sealing element 152 can be fitted to individual lipid-rich cell deposits to achieve an approximately air-tight seal, achieve the vacuum pressure for drawing tissue into the tissue-receiving cavity 158, maintain suction to hold the tissue, massage tissue (e.g., by altering pressure levels), and use little or no force to maintain contact between the applicator 102a and a patient.

The applicator 102a can further include one or more thermal devices 164 coupled to, embedded in, or otherwise in thermal communication with the temperature-controlled surface 160 of the cup 156. The thermal devices 164 can include, without limitation, one or more thermoelectric elements (e.g., Peltier-type elements), fluid-cooled elements, heat-exchanging units, or combinations thereof. In a cooling mode, fluid-cooled elements can cool the backside of the thermoelectric elements to keep the thermoelectric elements at or below a target temperature. In a heating mode, fluid-cooled elements can heat the backside of the thermoelectric elements to keep the thermoelectric elements at or above a target temperature. In some embodiments, the thermal devices 164 include only fluid-cooled elements or only non-fluid-cooled elements. The thermal devices 164 can be coupled to, embedded in, or associated with the cup 156. Although the illustrated embodiment has two thermal devices 164, in other embodiments the applicator 102a can have any desired number of thermal devices 164. The number, positions, configurations, and operating temperatures of the thermal devices 164 can be selected based on cooling/heating suitable for treatment, desired power consumption, or the like.

The applicator 102a can be used to cool a subcutaneous target region 166, e.g., by transferring heat from subcutaneous, lipid-rich tissue 168 via the cup 156 to the thermal devices 164. The temperature-controlled surface 160 can thermally contact an area of the subject's skin less than or equal to about 20 cm², 40 cm², 80 cm², 100 cm², 140 cm², 160 cm², 180 cm², 200 cm², 300 cm², 500 cm², or other suitable area. For example, the temperature-controlled surface area 160 can be, for example, equal to or less than 20 cm², 40 cm², 80 cm², 100 cm², 140 cm², 160 cm², 180 cm², 200 cm², 300 cm², or another suitable area. The temperature-controlled surface 160 can be cooled to a temperature equal to or less than a selected temperature (e.g., 5° C., 0° C., −2° C., −5° C., −7° C., −10° C., −15° C., −20° C., −25° C., etc.) to cool most of the skin surface 155 of the retained tissue. In one embodiment, most of the temperature-controlled surface 160 can be cooled to a temperature equal to or less than about 0° C., −2° C., −5° C., −10° C., or −15° C. In some embodiments, the temperature-controlled surface 160 is cooled to a temperature of about −11° C., the skin surface 155 is cooled to a temperature of about −10° C., and the subcutaneous target region 166 is cooled to temperatures within a range from about −8° C. to about 10° C. The cooled temperature of the subcutaneous target region 166 can vary based on the tissue depth, e.g., subcutaneous tissue within 1.5 mm of the skin surface 155 can be cooled to about −8° C., subcutaneous tissue within 11.5 mm of the skin surface 155 can be cooled to about 4° C., and subcutaneous tissue deeper than 11.5 mm can be cooled to about 10° C.

The heat extracted from the target region 166 can be carried away from the thermal devices 164 via a circulating coolant (not shown), as described in greater detail below. In some embodiments, the cooling treatment primarily affects lipid-rich cells in the target region 166 with little or no reduction or damage to non-lipid-rich cells in or near the region 166 (e.g., cells in the dermis 170 and/or epidermis 172).

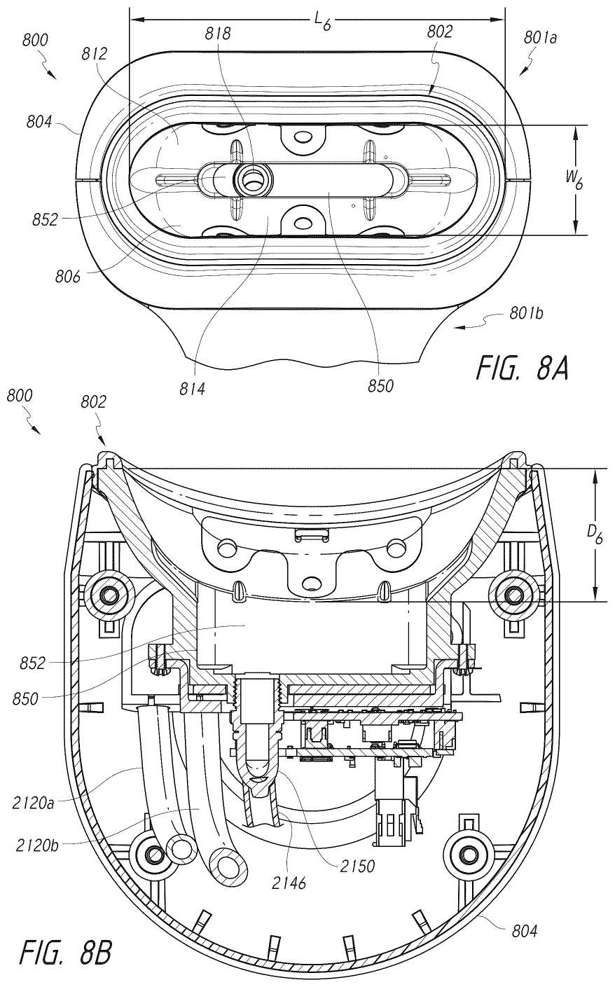

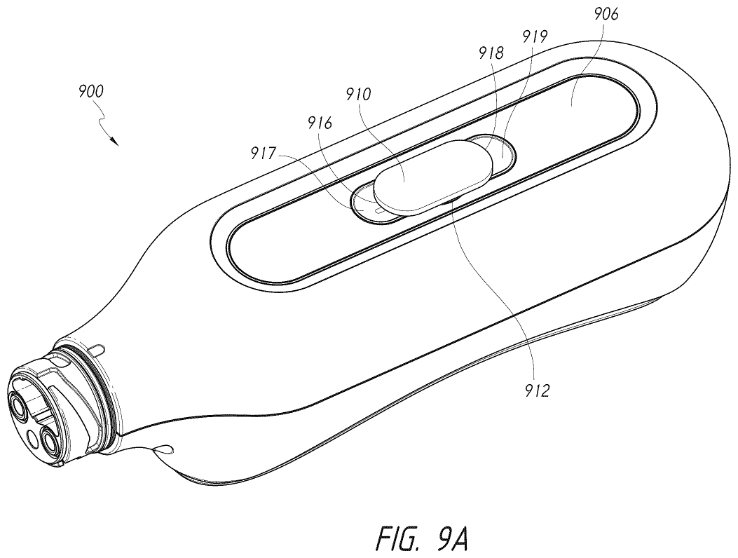

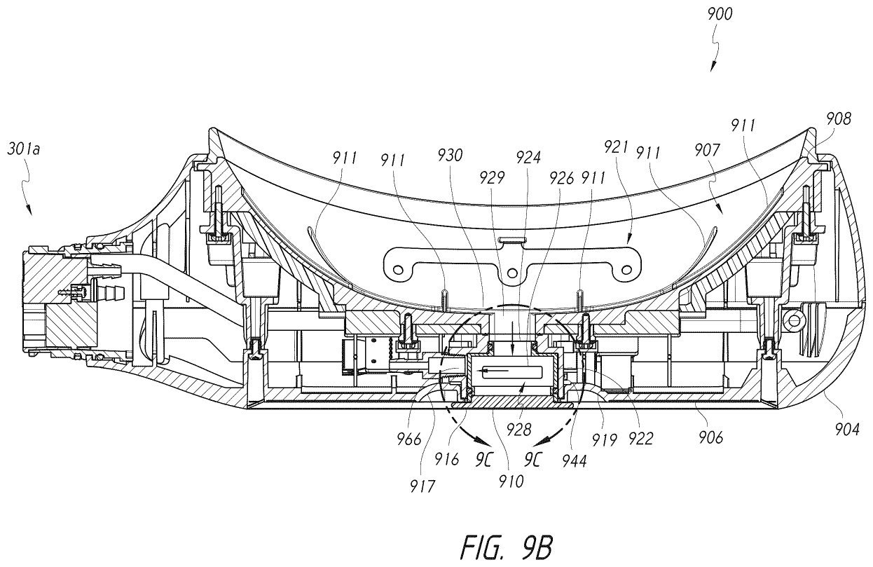

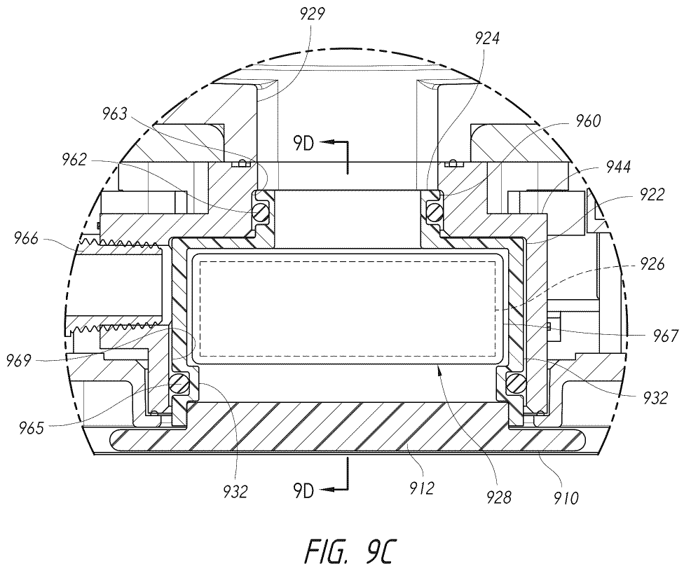

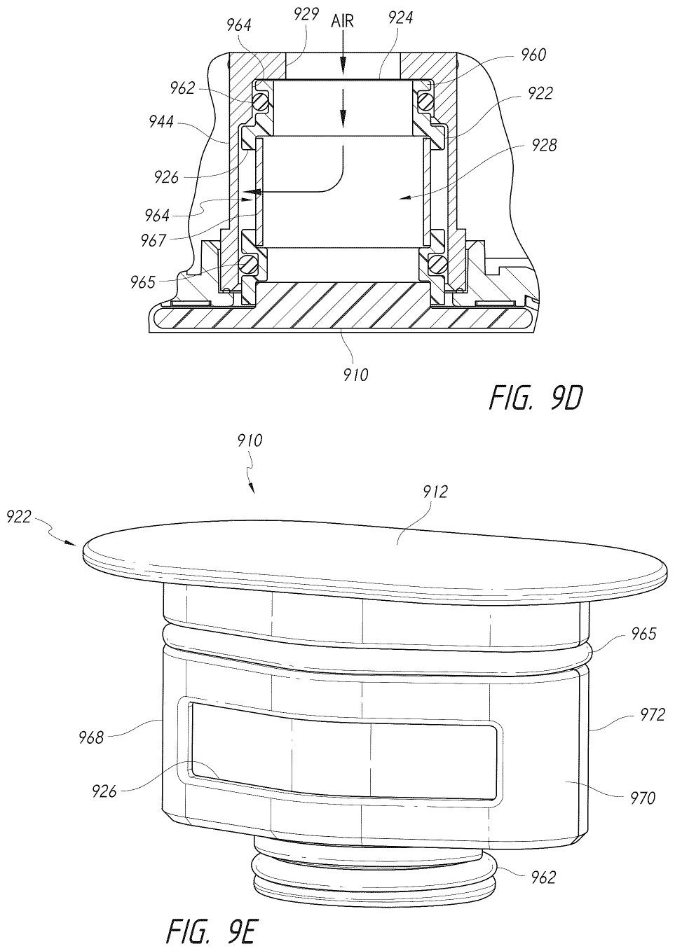

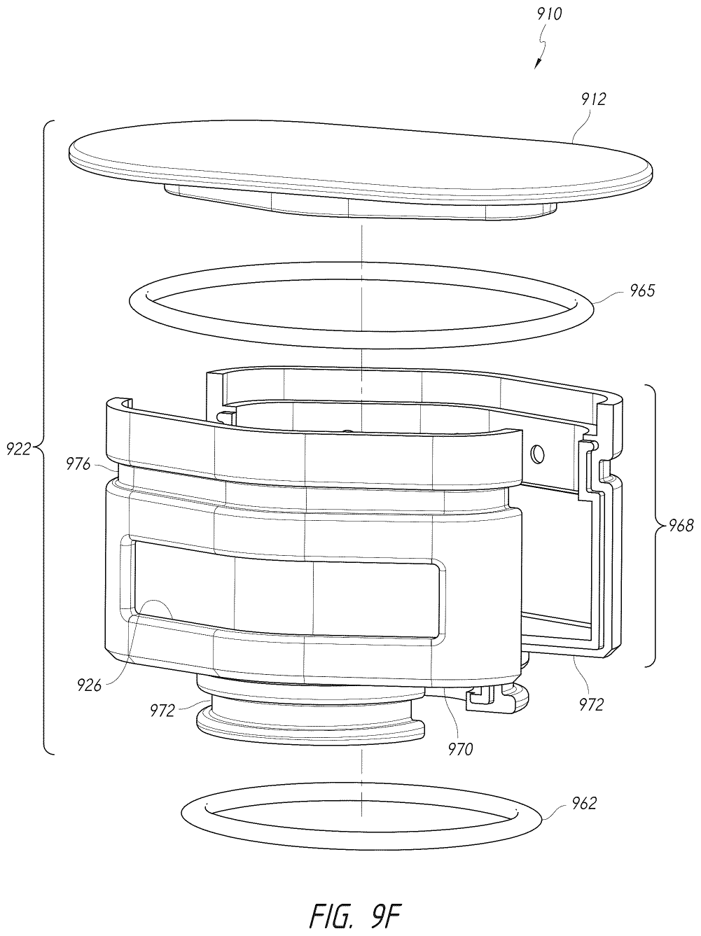

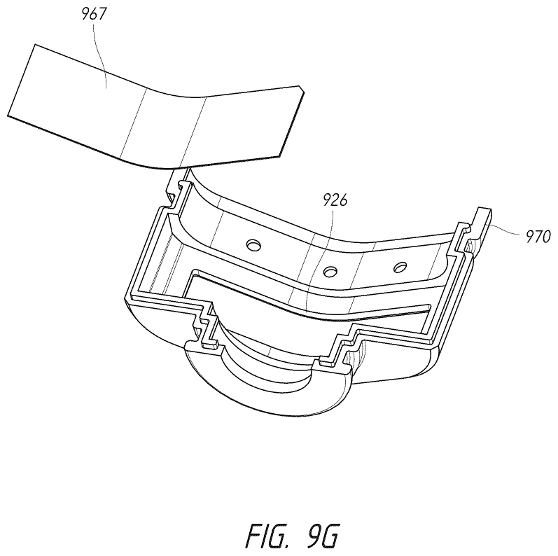

The applicator 102a can include a trap 165 that selectively captures substances (e.g., cryoprotectant gel, liquid, condensation, etc.) drawn into the vacuum port 162. The trap 165 can hold the captured substances away from the applicator-skin interface to maintain a high area of thermal contact and prevent the substances from reaching downstream components. The trap 165 can include a chamber 171, an outlet 173, and an air-permeable element 167 (e.g., an air-permeable and gel-impermeable membrane) covering the outlet 173. In some embodiments, the trap 165 functions as a gel trap. When the vacuum is started, air (indicated by arrows) can be drawn into and through the vacuum port 162. Gel 169 can also be drawn through the vacuum port 162 and into the trap 165. Air in the chamber 171 can flow through the air-permeable element 167 and into a passageway 177 between the trap 165 and a backside receiving feature or manifold 175. The air ultimately flows away from the applicator 102 via the connector 104A (FIG. 1A). The accumulated gel 169 is held away from heat flow paths between the cup 156 and the subject's tissue. The trap 165 is viewable from a backside of the applicator during treatment to confirm installation. The trap 165 can be emptied of accumulated gel 169 when the vacuum is stopped (e.g., between treatment sessions, after completion of a set of sessions, etc.). The number, configuration, holding capacity, and filtering capabilities of traps can be selected based on the procedure to be performed, and example traps are described in greater detail below and in connection with FIGS. 9A-9G.

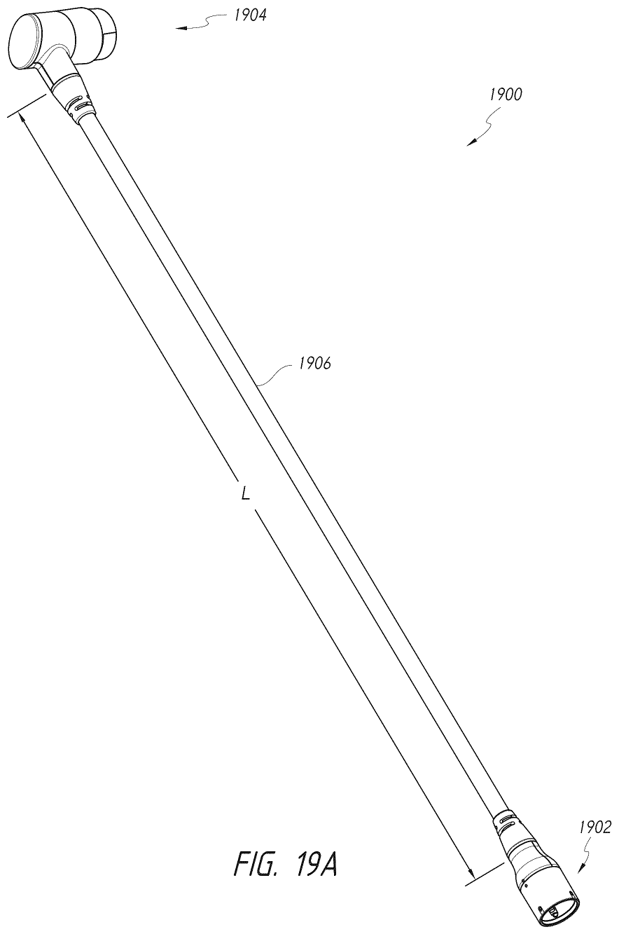

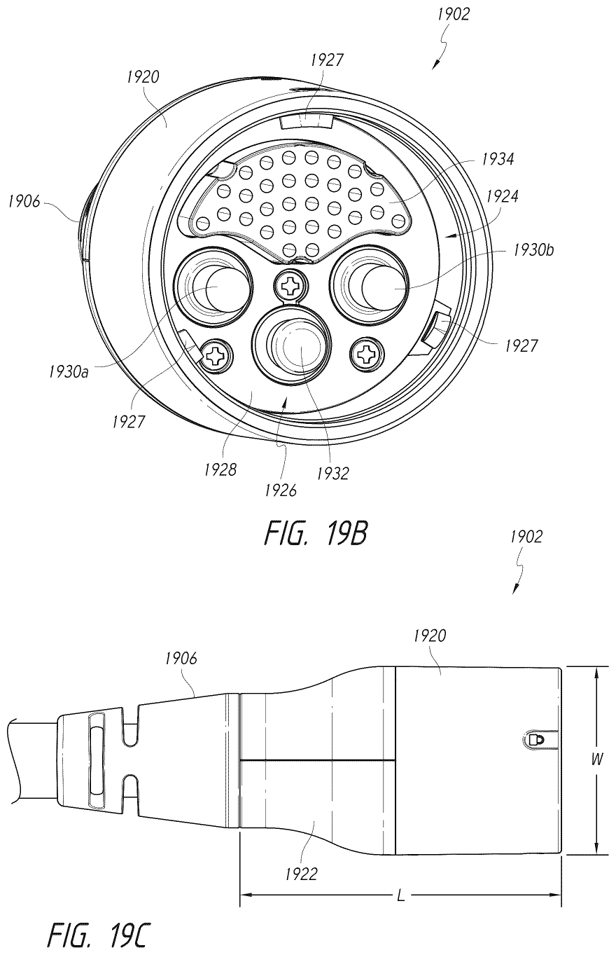

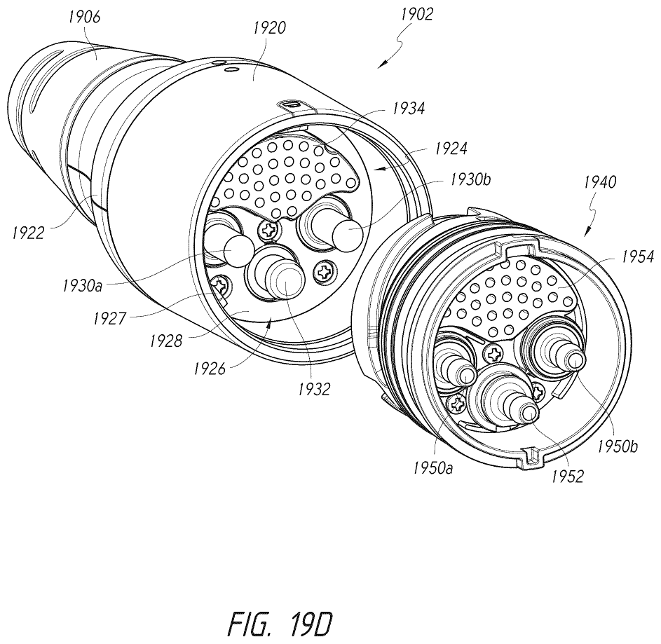

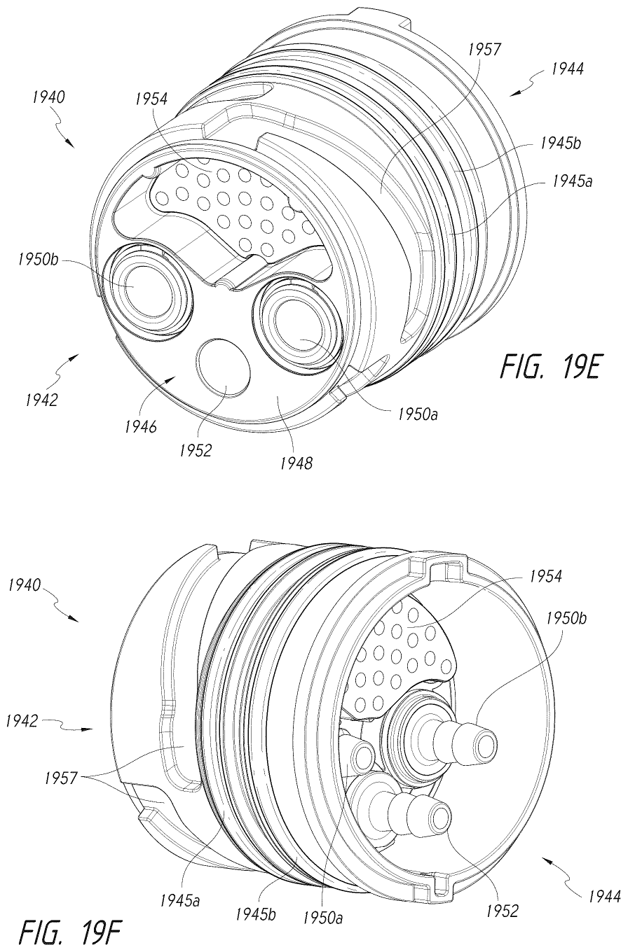

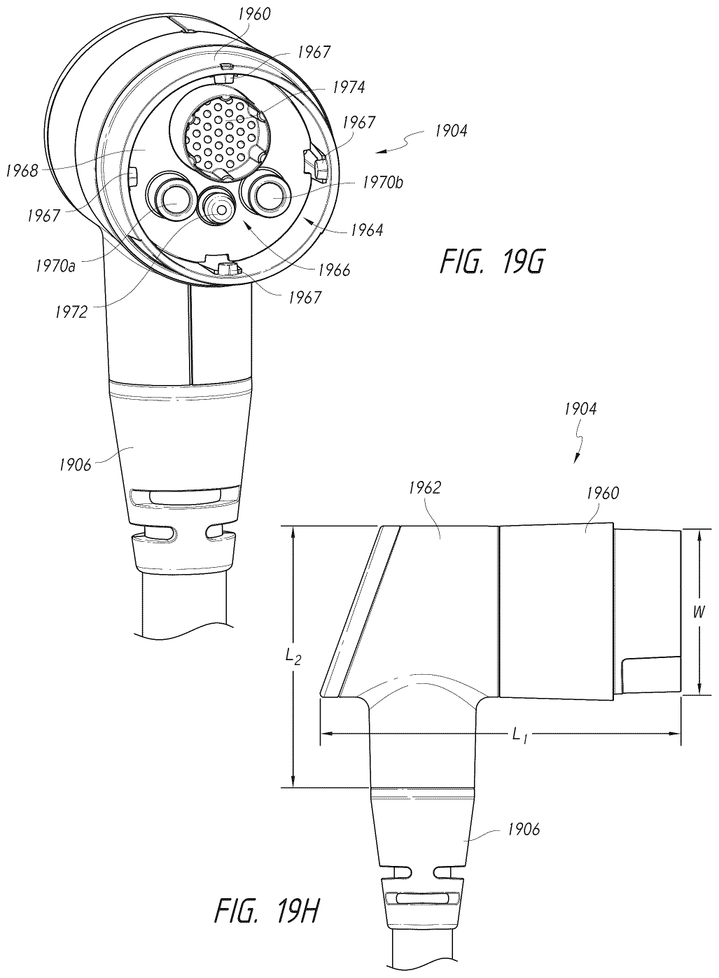

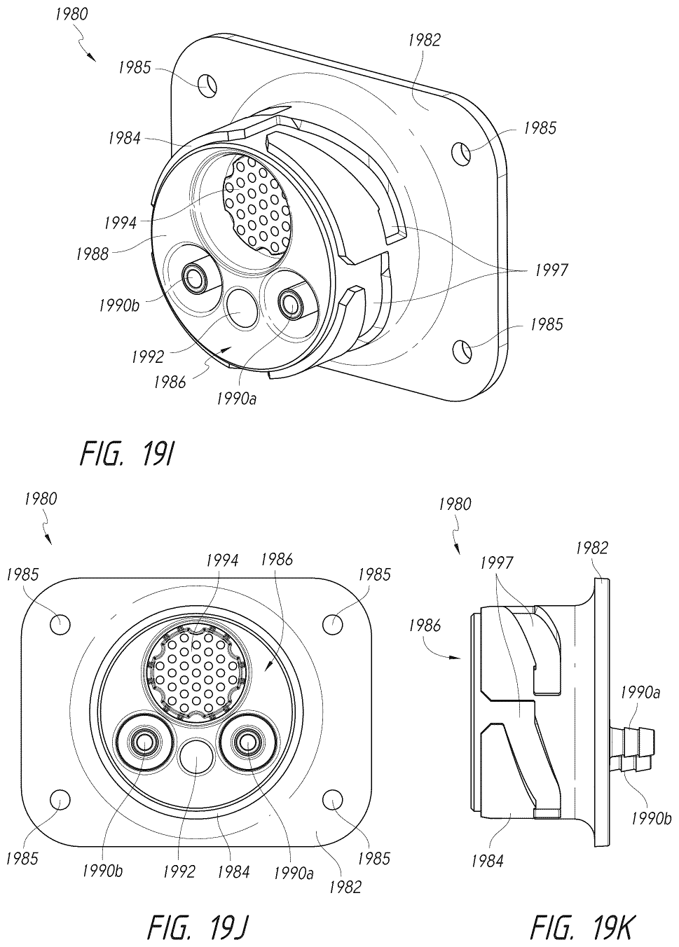

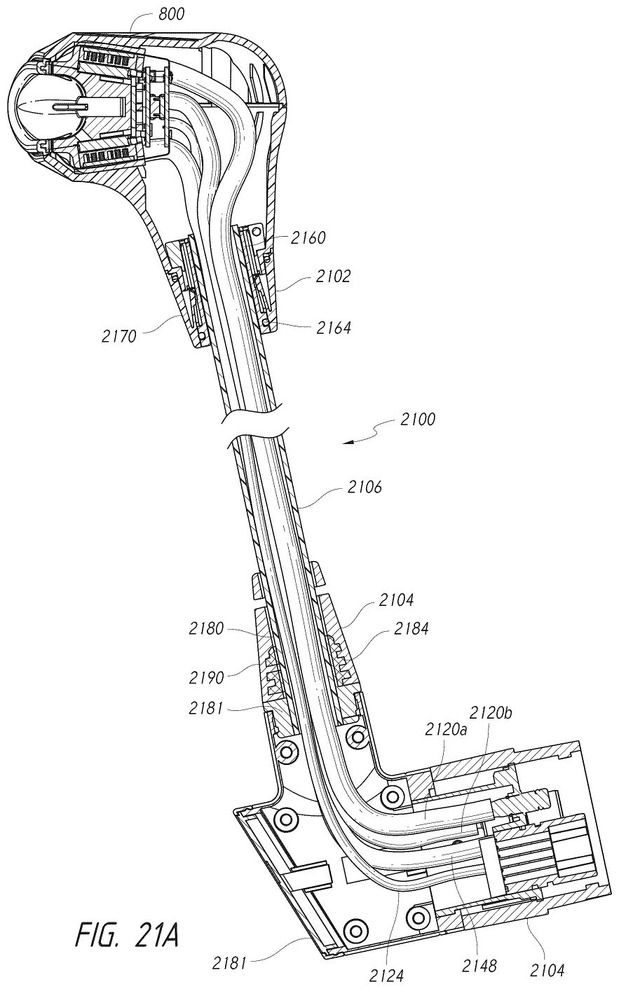

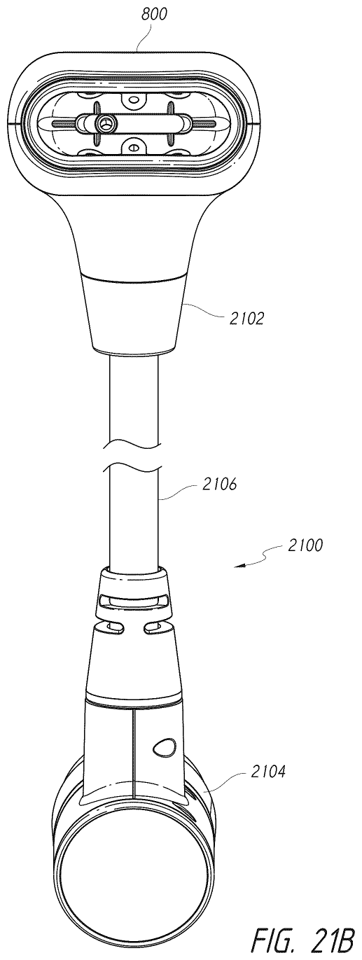

Referring again to FIG. 1A, the treatment system 100 includes a first connector 104a and a second connector 104b (collectively, “connectors 104”) that extend from a control unit or module 106 to the first applicator 102a and the second applicator 102b, respectively. The connectors 104 can provide suction for drawing tissue into the applicators 102, and can also deliver energy (e.g., electrical energy) and fluid (e.g., coolant) from the control unit 106 to the applicators 102. In some embodiments, each connector 104 is configured to releasably couple to the applicator 102 and/or the control unit 106 (e.g., via a bayonet connection). Additional examples of connectors are described in detail below in connection with FIGS. 19A-19K and 21A-21B.

FIG. 1C is a cross-sectional view of the first connector 104a and shows the connector 104a including a main body 179, a supply fluid line or lumen 180a (“supply fluid line 180a”), and a return fluid line or lumen 180b (“return fluid line 180b”). The main body 179 may be configured (via one or more adjustable joints) to “set” in place for the treatment of the subject 101. The supply and return fluid lines 180a, 180b can be conduits comprising, in whole or in part, polyethylene, polyvinyl chloride, polyurethane, and/or other materials that can accommodate circulating coolant, such as water, glycol, synthetic heat transfer fluid, oil, a refrigerant, and/or any other suitable heat conducting fluid for passing through fluid-cooled elements (e.g., thermal devices 164 of FIG. 1B), or other components. In one embodiment, each fluid line 180a, 180b can be a flexible hose surrounded by the main body 179.

The connector 104a can also include one or more electrical lines 112 for providing power to the applicator 102a and one or more control lines 116 for providing communication between the control unit 106 (FIG. 1A) and the applicator 102a (FIGS. 1A and 1B). The electrical lines 112 can provide power to the thermoelectric elements, sensors, and so forth. To provide suction, the connector 104a can include one or more vacuum lines 125. In various embodiments, the connector 104a can include a bundle of fluid conduits, a bundle of power lines, wired connections, vacuum lines, and other bundled and/or unbundled components selected to provide ergonomic comfort, minimize unwanted motion (and thus potential inefficient removal of heat from the subject), and/or to provide an aesthetic appearance to the treatment system 100.

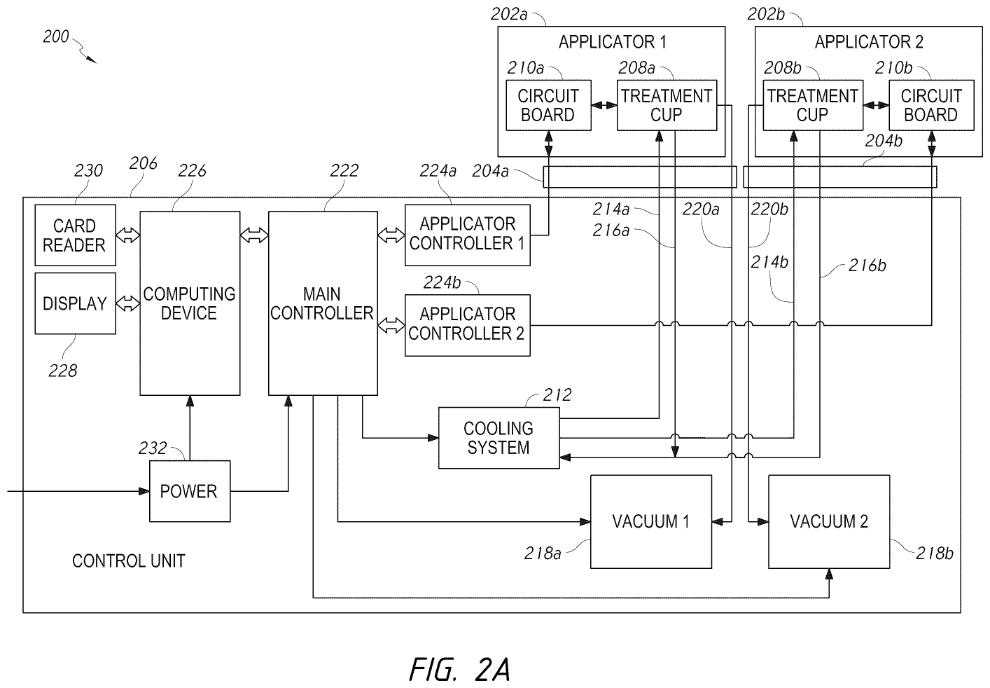

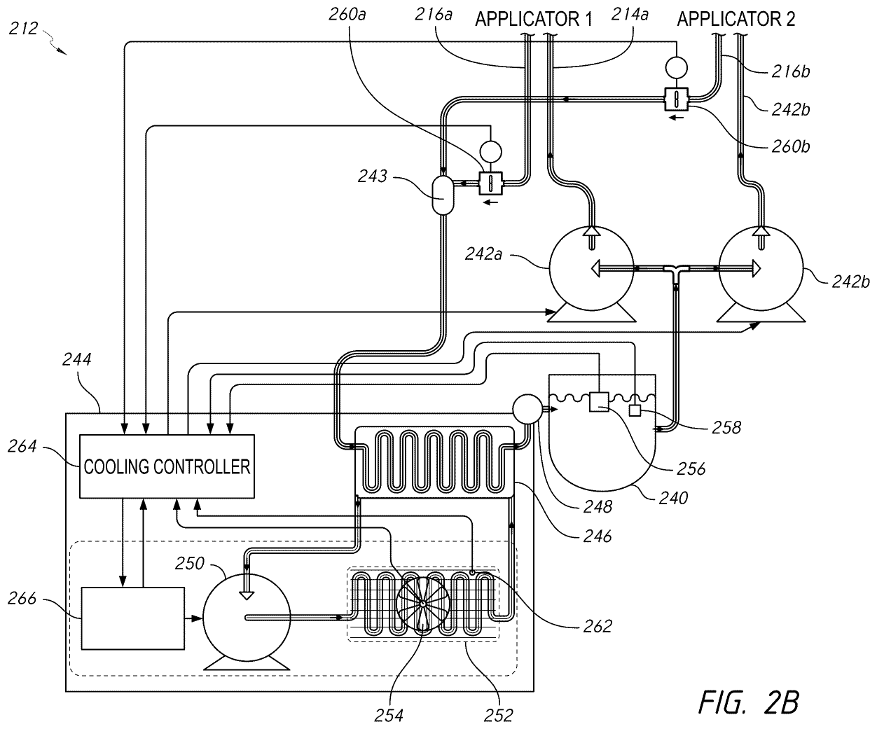

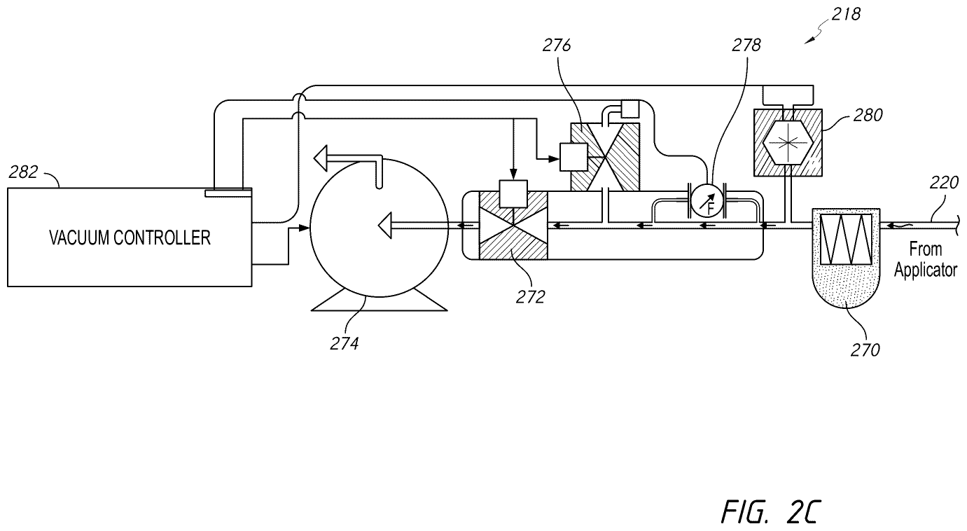

Referring again to FIG. 1A, the control unit 106 can include a cooling or fluid system 105 (illustrated in phantom line), a power supply 110 (illustrated in phantom line), and a controller 114 carried by a housing 124 with wheels 126. The cooling system 105 can include one or more fluid chambers, refrigeration units, cooling towers, thermoelectric chillers, heaters, or any other devices capable of controlling the temperature of coolant in the fluid chamber. The coolant can be continuously or intermittently delivered to the applicators 102 via the supply fluid line 180a (FIG. 1C) and can circulate through the applicators 102 to absorb heat. The coolant, which has absorbed heat, can flow from the applicators 102 back to the control unit 106 via the return fluid line 180b (FIG. 1C). The control unit 106 can have multiple refrigeration units, each cooling coolant from one of the applicators 102. For warming periods, the control unit 106 can heat the coolant that is circulated through the applicators 102. Alternatively, a municipal water supply (e.g., tap water) can be used in place of or in conjunction with the control unit 106. Additional examples of cooling systems are discussed below in connection with FIGS. 2A and 2B.

A pressurization device or vacuum system 123 (illustrated in phantom line) can provide suction to the applicator 102 via the vacuum line 125 (FIG. 1C) and can include one or more vacuum sources (e.g., pumps). Air pockets between the subject's tissue and the temperature-controlled surface 160 of the applicator 102a can impair heat transfer with the tissue and, if large enough, can affect treatment efficacy. The pressurization device 123 can provide a sufficient vacuum to eliminate such air gaps (e.g., large air gaps between the tissue and the temperature-controlled surface 160 of FIG. 1B) such that substantially no air gaps impair non-invasively cooling of the subject's subcutaneous lipid-rich cells to a treatment temperature. Additional examples of pressurization devices/vacuum systems are discussed below in connection with FIGS. 2A and 2C.

Air pressure can be controlled by one or more regulators located between the pressurization device 123 and the applicator 102. The control unit 106 can control the vacuum level to, for example, draw tissue into the applicator 102 while maintaining a desired level of comfort. If the vacuum level is too low, a liner assembly, gel pad, tissue, etc. may not be drawn adequately (or at all) into and/or held within the applicator 102. If the vacuum level is too high when preparing the applicator 102, a liner assembly can break (e.g., rupture, tear, etc.). If the vacuum level is too high during treatment, the patient can experience discomfort, bruising, or other complications. According to certain embodiments, approximately 0.5 inHg, 1 inHg, 2 inHg, 3 inHg, 5 inHg, 7 inHg, 8 inHg, 10 inHg, or 12 inHg vacuum is applied to draw or hold the liner assembly, tissue, etc. Other vacuum levels can be selected based on the characteristics of the tissue, desired level of comfort, and vacuum leakage rates. Vacuum leak rates of the applicator 102 can be equal to or less than about 0.2 LPM, 0.5 LPM, 1 LPM, or 2 LPM at the pressure levels disclosed herein. For example, the vacuum leak rate can be equal to or less than about 0.2 LPM at 8 inHg, 0.5 LPM at 8 inHg, 1 LPM at 8 inHg, or 2 LPM at 8 inHg. The configuration of the pressurization device 123 and applicator 102 can be selected based on the desired vacuum levels, leakage rates, and other operating parameters.

The power supply 110 can provide a direct current voltage for powering electrical elements of the applicators 102 via the line 112 (FIG. 1C). The electrical elements can be thermal devices, sensors, actuators, controllers (e.g., a controller integrated into the applicators 102), or the like. An operator can use an input/output device 118 (e.g., a screen) of the controller 114 to control operation of the treatment system 100, and the input/output device 118 can display the state of operation of the treatment system 100 and/or progress of a treatment protocol. In some embodiments, the controller 114 can exchange data with the applicator 102 via the line (e.g., line 116 of FIG. 1C), a wireless communication link, or an optical communication link and can monitor and adjust treatment based on, without limitation, one or more treatment profiles and/or patient-specific treatment plans, such as those described, for example, in commonly assigned U.S. Pat. No. 8,275,442. The controller 114 can contain instructions to perform the treatment profiles and/or patient-specific treatment plans, which can include one or more segments, and each segment can include temperature profiles, vacuum levels, and/or specified durations (e.g., 1 minute, 5 minutes, 10 minutes, 20 minutes, 30 minutes, 1 hour, 2 hours, etc.). For example, the controller 114 can be programmed to cause the pressurization device to operate to pull tissue into the applicator. After tissue draw, the pressurization device can operate to hold the subject's skin in thermal contact with appropriate features while the cup 156 (FIG. 1B) conductively cools tissue. If a sensor detects tissue moving out of thermal contact with the cup 156, the vacuum can be increased to reestablish suitable thermal contact. In some embodiments, the controller 114 is programmed to cause the pressurization device 123 to provide a sufficient vacuum to keep substantially all of each region of the temperature-controlled surface 160 (FIG. 1B) between air-egress features in thermal contact with the subject's skin. This provides a relatively large contact interface for efficient heat transfer with the target tissue.

Different vacuum levels can be utilized during treatment sessions. For example, relatively strong vacuums can be used to pull the subject's tissue into the applicator 102. A weaker vacuum can be maintained to hold the subject's tissue against the thermally conductive surface. If suitable thermal contact is not maintained (e.g., the subject's skin moves away from the thermally conductive surface), the vacuum level can be increased to reestablish suitable thermal contact. In other procedures, a generally constant vacuum level can be used throughout the treatment session.

In some embodiments, a treatment profile includes specific profiles for each applicator 102 to concurrently or sequentially treat multiple treatment sites, including, but not limited to, sites along the subject's torso, abdomen, legs, buttock, legs, face and/or neck (e.g., submental sites, submandibular sites, etc.), knees, back, arms, ankle region, or other treatment sites. The vacuum levels can be selected based on the configuration of the cup. Strong vacuum levels can be used with relatively deep cups whereas weak vacuum levels can be used with relatively shallow cups. The vacuum level and cup configuration can be selected based on the treatment site and desired volume of tissue to be treated. In some embodiments, the controller 114 can be incorporated into the applicators 102 or another component of the treatment system 100. Additional examples of control units and controllers are described below in connection with FIGS. 2A, 22A-22C, and 24.

### B. Treatment System

FIG. 2A is a schematic block diagram illustrating a treatment system 200 configured in accordance with embodiments of the present technology. The components of the treatment system 200 can be identical or generally similar to the components of the treatment system 100. For example, as shown in FIG. 2A, the treatment system 200 includes a first applicator 202a and a second applicator 202b (collectively, “applicators 202”), a first connector 204a and a second connector 204b (collectively, “connector 204”), and a control unit 206. The first applicator 202a is coupled to the control unit 206 via the first connector 204a, and the second applicator 202b is coupled to the control unit 206 via the second connector 204b. Each applicator 202 includes a respective treatment cup 208 (e.g., first and second treatment cups 208a, 208b) for receiving and cooling a patient's tissue. The treatment cups 208 can include and/or be coupled to thermal devices (not shown) configured to draw heat from the patient's tissue. Each treatment cup 208 can be coupled to a respective circuit board 210 (e.g., first and second circuit boards 210a, 210b) including electronic components for monitoring the treatment applied to the tissue and routing control and/or power signals, as described in greater detail below.

The control unit 206 includes various components for controlling the treatment applied to the patient's tissue via the applicators 202. In some embodiments, for example, the control unit 206 includes a cooling system or unit 212 operably coupled to the treatment cups 208 of the applicators 202. The cooling system 212 can be identical or similar to the cooling system 105 of FIG. 1A. The cooling system 212 can be configured to deliver a coolant to the applicators 202 (e.g., via supply fluid lines 214a, 214b) that circulates through the system 200 to absorb heat from the patient's tissue. The heated coolant can flow from the applicators 102 back to the cooling system 212 (e.g., via return fluid lines 216a, 216b). The cooling system 212 can reduce the temperature of the returned coolant and recirculate the coolant to the applicators 202. Additional details of the cooling system 212 are provided further below in connection with FIG. 2B.

The control unit 206 optionally includes a first vacuum system or unit 218a operably coupled to the first treatment cup 208a via a first vacuum line 220a, and a second vacuum system or unit 218b operably coupled to the second treatment cup 208b via a second vacuum line 220b. Although the first and second vacuum systems 218a, 218b (collectively, “vacuum systems 218”) are illustrated as separate components, in other embodiments the first and second vacuum systems 218a, 218b can be replaced with a single vacuum system for both applicators 202. Similar to the pressurization device 123 of FIG. 1A, the vacuum systems 218 can provide suction to draw the patient's tissue into contact with the surfaces of the treatment cups 208 for more efficient cooling. In some embodiments, each applicator 202 has a vacuum-based tissue retention factor that may be expressed as a ratio of a treatment area of the applicator 202 to the weight of the applicator 202. The vacuum-based tissue retention factor can be sufficiently high such that the applicator 202 can remain secured to the subject only via the applied vacuum. For example, the vacuum-based tissue retention factor can be greater than or equal to 5 square inches per lb, 6 square inches/lb, 7 square inches/lb, 8 square inches/lb, 9 square inches/lb, 10 square inches/lb, 11 square inches/lb, 12 square inches/lb, 13 square inches/lb, 14 square inches/lb, 15 square inches/lb, 16 square inches/lb, 17 square inches/lb, 18 square inches/lb, 19 square inches/lb, or 20 square inches/lb. Additional details of the vacuum systems 218 are provided further below in connection with FIG. 2C.

The control unit 206 can include various hardware and software components for controlling the applicators 202, cooling system 212, and vacuum systems 218. In the illustrated embodiment, for example, the control unit 206 includes a main controller 222, a first applicator controller 224a, and a second applicator controller 224b. The main controller 222 can be operably coupled to the cooling system 212, vacuum systems 218, and the first and second applicator controllers 224a, 224b (collectively, “applicator controllers 224”) to control the operation thereof. In some embodiments, the main controller 222 is electrically coupled to each of these components to provide power and control signals thereto, and can also receive status signals, sensor data (e.g., moisture data, flow rates, etc.), and/or other data from the components. For example, the main controller 222 can send control signals to the cooling system 212 to control the amount and/or rate of cooling, coolant flow rates, and/or other operational parameters. The main controller 222 can also receive sensor data from the cooling system 212 (e.g., temperature data, flow data, coolant level data) to assess the status of the cooling system 212. As another example, the main controller 222 can independently send control signals to the first and second vacuum systems 218a, 218b to control the amount of vacuum applied via the first and second applicators 202a, 202b, respectively. The main controller 222 can also receive sensor data from the first and/or second vacuum systems 218a, 218b (e.g., pressure data, flow data, etc.) to determine whether a suitable amount of pressure is being applied, or whether the pressure level should be adjusted.

In the illustrated embodiment, the main controller 222 is not directly connected to the circuit boards 210, and is instead indirectly coupled via the respective applicator controllers 224. As described in greater detail below, the circuit boards 210 located within the applicators 202 can be configured to perform a limited set of operations, such as routing data and/or signals between the applicator controllers 224 and the applicator components associated with the treatment cups 208 (e.g., thermal devices, sensors, etc.). The remaining operations (e.g., data processing, control of applicator components, etc.) can be performed by the main controller 222 and/or the applicator controllers 224. In some embodiments, the first and second applicator controllers 224a, 224b can be operated independently from each other so that the first and second applicators 202a, 202b can apply different treatment profiles to the patient (e.g., based on the particular patient location to be treated).

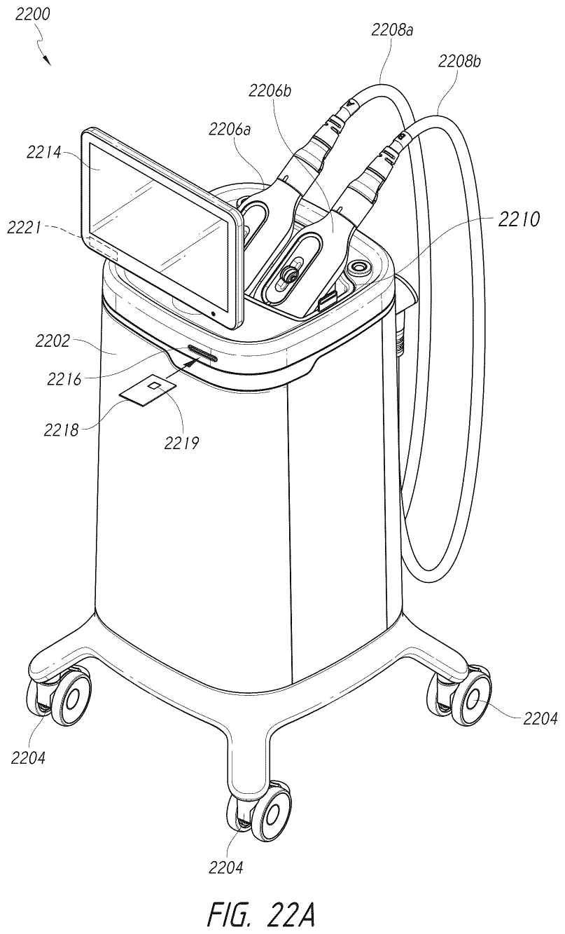

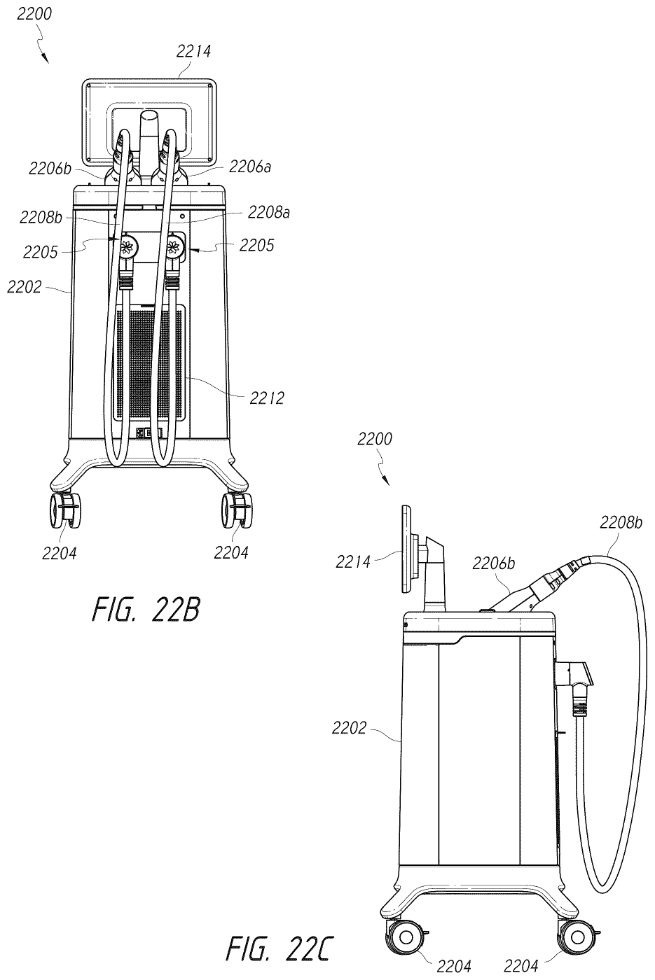

The treatment system 200 further includes a computing device 226. The computing device 226 can be configured to receive input from an operator of the treatment system 200 via user interface elements such as a display 228 (e.g., a monitor or touchscreen). The computing device 226 can transmit the user input to the main controller 222, which converts the user input into control signals for operating the various system components (e.g., applicators 202, cooling system 212, and/or vacuum systems 218). Conversely, data received from the system components can be transmitted by the main controller 222 to the computing device 226 and be displayed to the user via the display 228. Optionally, the computing device 226 can be operably coupled to a card reader 230. The card reader 230 can be configured to receive a card that provides security information, treatment profile information, patient information, and/or other information relevant to the operation of the treatment system 200, as described in greater detail below in connection with FIGS. 22A-22C.

The operation of the treatment system 200 can be powered by a power system or unit 232. The power system 232 can receive power from an external power source such as an electrical wall outlet (not shown), and can be electrically coupled to the main controller 222 and computing device 226 to provide power thereto. The external power source can have a line voltage within a range from 100 V to 240 V, such as 100 V, 120 V, 200 V, 220 V, or 240 V. The main controller 222 can provide power to the remaining components of the treatment system 200 (e.g., circuit boards 210, cooling system 212, vacuum systems 218, and/or applicator controllers 224). The power system 232 can be configured to allow the treatment system 200 to operate with a variety of different voltages from the external power source. For example, the power system 232 can include a transformer circuit that automatically detects the line voltage from the external power source (e.g., 100-120, 200-240 V at 50-60 Hz) and converts the line voltage to the system voltages used by the system components (e.g., 24 V for the main controller 222, 12 V for the computing device 226). In some embodiments, the transformer circuit can automatically measure the input line voltage and AC cycles, and convert the input into a constant output (e.g., 230 V at 50-60 Hz).

It will be appreciated that the treatment system 200 can be configured in many different ways. In other embodiments, for example, some of the components of the treatment system 200 can be combined with each other (e.g., the vacuum systems 218, the main controller 222, and applicator controllers 224). Alternatively, some of the components of the treatment system 200 can be provided as discrete, separate components (e.g., the main controller 222 can be separated into two or more discrete modules). Additionally, some of the components of the treatment system 200 can be omitted in other embodiments (e.g., the second applicator 202b, second connector 240b, and second vacuum system 218b). The treatment system 200 can also include components known to those of skill in the art that are omitted from FIG. 2A merely for purposes of clarity.

### C. Cooling System

FIG. 2B is a schematic diagram of the cooling system 212 of the treatment system 200 of FIG. 2A. The cooling system 212 can be configured to remove heat from a patient via at least one applicator (e.g., applicators 202 of FIG. 2A) during a course of a cooling treatment applied to the patient. Optionally, the cooling system 212 can also remove heat from electronics or other components of the applicators 202 and/or treatment system 200 (e.g., circuit boards 210 of FIG. 2A). In some embodiments, the majority of the heat removed from the applicator 202 originates from the patient's tissue, rather than from internal components of the applicator 202 (e.g., at least 70%, 80%, 90%, 95% of the heat originates from the patient's tissue). In some embodiments, heat produced by drivers, control circuitry, etc. can be generated remotely from the applicator 202. For example, as discussed in greater detail below, applicator controllers or drivers can be part of the control unit 106 such that a majority of heat (e.g., at least 70%, 80%, 90%, or 95% of the heat) produced by circuitry (e.g., drive circuitry, control circuitry, etc.) is generated within the control unit 106 and away from the applicators 202. In some embodiments, a ratio of heat absorbed by the applicator 202 from the subject's tissue to the heat actively removed (e.g., via circulating coolant) from the applicator by the treatment system is equal to greater than 0.7, 0.8, 0.9, or 0.95 during a portion or most of the treatment. The removed heat can be transferred to the room environment in which the treatment system 200 is operating.

The cooling system 212 can be configured in many different ways. In some embodiments, for example, the cooling system 212 includes a fluid chamber 240 for storing a coolant. The cooling system 212 can include a first coolant pump 242a for circulating the coolant to the first applicator 202a (FIG. 2A) via the supply fluid line 214a, and a second coolant pump 242b for circulating the coolant to the second applicator 202b (FIG. 2A) via the supply fluid line 214b. Optionally, the first and second coolant pumps 242a, 242b can be replaced with a single coolant pump. The coolant can be circulated through the applicators 202 to absorb heat from the patient. The heated coolant then returns to the cooling system 212 via the return fluid lines 216a, 216b, respectively, for cooling. In embodiments where the multiple applicators 202 are used concurrently, the cooling system can cool the coolant from each applicator 202 independently or together. For example, the cooling system 212 can include a manifold 243 for combining the coolant from the return fluid lines 216a, 216b before cooling. In some embodiments, the cooling system 212 includes a vapor compression subsystem 244 for cooling the heated coolant. The vapor compression subsystem 244 can include components such as pumps, evaporators, condensers, fans, compressors, refrigerants, etc. For example, in the illustrated embodiment, the heated coolant flows through an evaporator 246, where the heat is transferred from the coolant to a refrigerant (e.g., R-134a). Once cooled, the cooled coolant can be returned to the fluid chamber 240 for re-circulation. Optionally, a filter 248 can be used to filter the coolant before it re-enters the fluid chamber 240.

The vapor compression subsystem 244 can further include a compressor 250, a condenser 252, and a fan 254. The heated refrigerant from the evaporator 246 can be circulated through the compressor 250 and the condenser 252 before returning to the evaporator 246. The compressor 250 can be a fixed speed compressor or a variable speed compressor. A fixed speed compressor may only have two compressor speed/power settings (e.g., on (100% power) and off (0% power)), while a variable speed compressor may have multiple speed/power settings (e.g., within a range from 0% power to 100% power). For example, the cooling system can have a variable speed compressor having power settings that are variable within a range from 40% power to 100% power in order to provide different cooling capacities. The power setting of the variable speed compressor can be varied based on the particular treatment procedure, applicator, and/or target efficiency. The use of a variable speed compressor may be advantageous for improving efficiency and reducing power consumption.

The cooling system 212 can include various types of sensors (e.g., flow sensors, temperature sensors, fluid level sensors) to monitor coolant circulation and/or temperature at various points in the system (e.g., at the fluid supply and/or return lines, fluid reservoir, etc.). For example, the cooling system 212 can include a fluid level sensor 256 and/or a fluid temperature sensor 258 in the fluid chamber 240. The cooling system 212 can also include first and second flow sensors 260a, 260b at the return fluid lines 216a, 216b. The cooling system 212 can also include an air temperature sensor 262 at the condenser 252.

In some embodiments, the cooling system 212 includes a cooling controller 264 (e.g., a microcontroller). The cooling controller 264 can be configured to receive data from the various sensors, and output power and/or control signals for various components such as the first and second coolant pumps 242a, 242b, the compressor 250, and the fan 254. Optionally, the cooling controller 264 can be operably coupled to a compressor controller 266 which controls the operation of the compressor 250 and receives status signals from the compressor 250.

In some embodiments, the cooling controller 264 is configured to anticipate the heating load on the system 212 and adjust the compressor speed accordingly. For example, the compressor speed can be increased if a relatively high heating load is expected (e.g., for multi-applicator procedures and/or procedure using an applicator with a relatively large treatment surface area). The control algorithm for the variable compressor speed can provide non-proportional cooling for managing peak cooling. The cooling controller 264 can also regulate operations of the fan 254 to reduce system noise.

The cooling system 212 can be configured to operate with various types of coolants, such water, a water/ethylene glycol mixture, a water/propylene glycol mixture, a water/methanol mixture, or any other suitable coolant. The cooling system 212 can be configured to maintain the coolant at a target temperature during operation of the treatment system 200. The target temperature can be less than or equal to 0° C., −5° C., −10° C., or −15° C. The cooling system 212 can take approximately 10 minutes from the start of the treatment procedure to reach steady state. During operation, the coolant can be circulated through the cooling system 212 at a flow rate within a range from 0.8 LPM to 1.2 LPM. The fluid supply and return lines 214, 216 for circulating coolant to and from the applicators 202 can have an inner diameter of approximately 0.187 inches.

In some embodiments, the cooling system 212 is configured to cool the applicator surface at a rate within a range from 0.1° C./s to 5° C./s, or 0.2° C./s to 3° C./s. For example, the cooling rate can be 0.1° C./s, 0.2° C./s, 0.3° C./s, 0.4° C./s, 0.5° C./s, 0.6° C./s, 0.7° C./s, 0.8° C./s, 0.9° C./s, 1° C./s, 1.5° C./s, 2° C./s, 2.5° C./s, 3° C./s, 3.5° C./s, 4° C./s, 4.5° C./s, or 5° C./s. The cooling rate can be measured based on temperatures of the applicator surface during the initial cooling phase (e.g., within the first 10 minutes of cooling). The transient rate of heat removal from the applicator 202 and/or patient (e.g., the rate upon initial contact) can be greater than or equal to 200 W, such as at least 210 W, 220 W, 230 W, 240 W, 250 W, 260 W, 270 W, 280 W, 290 W, 300 W, or more. The steady state rate of heat removal from the applicator 202 and/or patient can be greater than or equal to 150 W, such as at least 160 W, 170 W, 180 W, 190 W, 200 W, 210 W, 220 W, 230 W, 240 W, 250 W, or more. The efficiency of the cooling system 212 (e.g., as expressed as the ratio between the heat removal rate and power usage) can be greater than or equal to 75%, or within a range from 50% to 95%. For example, the efficiency can be at least 50%, 60%, 70%, 75%, 80%, 85%, 90%, or 95%. The improved efficiency of the cooling system 212 can reduce the amount of heating of the surrounding environment during the treatment procedure.

In some embodiments, the cooling system 212 is configured to precool the coolant to the target temperature before starting the treatment procedure, e.g., to avoid pumping excess heat into the room during the start of the procedure. Precooling can be performed on a small volume of coolant using a TEC-based system. The treatment procedure can then be initiated using the chilled coolant.

### D. Vacuum System

FIG. 2C is a schematic diagram of a vacuum system 218 of the treatment system 200 of FIG. 2A. The vacuum system 218 is configured to apply vacuum to a patient's tissue during the course of a cooling treatment applied to the patient. Optionally, the vacuum can also be applied after the cooling treatment, e.g., to deliver a post-treatment vacuum massage.

As previously described with respect to FIG. 2A, each applicator 202 can be connected to a respective independent vacuum system 218 via a respective vacuum line 220. The vacuum line 220 can have an inner diameter of approximately 0.187 inches. In some embodiments, the vacuum system 218 further includes a fluid trap 270 (e.g., located within the control unit 206 of FIG. 2A) for trapping and/or removing fluid that enters the vacuum line 220 (e.g., gel, water condensed on the applicator surface and/or other system components, residue on the applicator from prior cleaning, etc.) and which is not otherwise trapped by a gel trap located in the applicator 202 (e.g., trap 165 of FIG. 1B). The use of a fluid trap 270 in the control unit 206 can be beneficial for improving vacuum performance, reducing maintenance frequency, and/or increasing the lifetime of the vacuum system 218. The fluid trap 270 can include one or more membranes, filters, valves, and/or other components configured to capture gel, liquid (e.g., water), or other contaminants in the vacuum line 220.

After exiting the fluid trap 270, the air passes through a proportional valve 272 and a vacuum pump 274, and exits the vacuum system 218. Optionally, the vacuum system 218 can include a bleed valve 276 between the fluid trap 270 and proportional valve 272. In some embodiments, the vacuum system 218 is a single-stage vacuum system (e.g., includes a single proportional valve 272 between the vacuum pump 274 and the applicator 202). The vacuum pump 274 can be configured to produce an air flow rate that is sufficiently high to rapidly evacuate air from the treatment system 200 (e.g., tubing, gel traps, etc.). For example, the air flow rate (e.g., as measured at the pump 274) can be at least 10 LPM, 15 LPM, or 20 LPM.

In some embodiments, the vacuum system 218 is configured to rapidly reach and maintain a target vacuum pressure with little or no oscillation (e.g., little or no overshoot and/or undershoot of the target pressure). The target vacuum pressure can be within a range from 3 inHg to 12 inHg, such as 8 inHg. The amount of time to reach the target vacuum pressure can be less than or equal to 5 seconds, 4 seconds, 3 seconds, 2 seconds, or 1 second. The amount of overshoot can be less than or equal to 20%, 15%, 10%, or 5% of the target vacuum pressure. The amount of undershoot can be less than or equal to 20%, 15%, 10%, or 5% of the target vacuum pressure. The dampening ratio of the overshoot to undershoot (e.g., upon initial vacuum draw and/or after a disturbance to the applied vacuum) can be within a range from 0.3 to 0.7, or approximately 0.7, 0.5, or 0.3. In some embodiments, for example, the vacuum system 218 reaches the target pressure in no more than 3 seconds with no more than 20% overshoot/undershoot.

Additionally, the vacuum system 218 can be configured to maintain the target vacuum pressure during the treatment procedure with little or no pressure drop or loss. In some embodiments, the total pressure drop or loss is no more than 50%, 40%, 30%, 20%, 10%, or 5% of the target pressure value. For example, the total pressure drop and/or loss across the vacuum system 218 can be no more than 3 inHg for a flow rate of 15 LPM. As described in greater detail below, the fittings between the vacuum system 218 and the other components of the treatment system 200 (e.g., between the connector 204 of FIG. 2A) can be configured to reduce or minimize leaks and/or other sources of pressure loss. The vacuum system 218 can also be configured to maintain a substantially constant amount of pressure, while avoiding excessively high and/or low vacuum pressures. For example, during operation of the vacuum system 218 (e.g., while vacuum pressure is being applied to the patient's tissue), the maximum vacuum pressure can be less than or equal to 12 inHg, and the minimum pressure can be greater than or equal to 3 inHg.

The vacuum system 218 can include various types of sensors (e.g., pressure sensors, flow sensors) to detect whether the applied vacuum pressure is too high or too low. In some embodiments, for example, the vacuum system 218 can include at least one sensor 278 configured to monitor air flow within the vacuum system 218. The vacuum system 218 can use the flow measurements to reliably detect conditions that may lead to “pop off” (e.g., vacuum pressure too low), “pop on” (e.g., vacuum pressure too high), leaks, or an improper seal between the applicator and the patient tissue. Pop off may occur if the vacuum pressure is less than a particular value (e.g., a value of 3 inHg, or within a range from 3 inHg to 7 inHg) for a certain time period (e.g., at least 3 seconds). Pop on may occur if the vacuum pressure is greater than a particular value (e.g., a value of 7 inHg, or within a range from 7 inHg to 12 inHg) for a particular time period (e.g., at least 3 seconds). Optionally, the sensor 278 can be located along the portion of the vacuum line near the vacuum pump 274, such as between the proportional valve 272 and the fluid trap 270. The flow-based techniques described herein for detecting pop off/pop on may be more robust and accurate than other techniques (e.g., pressure-based detection), and can be used to avoid vacuum conditions that are likely to adversely affect patient treatment. In some embodiments, the sensor 278 is configured to determine air flow based on pressure measurements (e.g., by calculating flow rate based on the pressure drop between two spaced-part pressure sensors). In other embodiments, the sensor 278 can directly measure air flow (e.g., by directly detecting a mass or volume rate of air flow). Optionally, the vacuum system 218 can also include a sensor 280 configured to measure vacuum pressure between the fluid trap 270 and the flow sensor 278.

The vacuum system 218 can also include a vacuum controller 282 (e.g., a microcontroller) for monitoring and controlling operation of the various components (e.g., vacuum pump 274, proportional valve 272, and/or bleed valve 276). The sensor(s) of the vacuum system 218 (e.g., sensor 278, 280, etc.) can provide feedback to a vacuum controller 282 to monitor and maintain the vacuum pressure applied by the vacuum system 218. Optionally, if the sensor data indicates that a malfunction has or is likely to occur (e.g., pop off, pop on, leaks, etc.), the vacuum controller 282 can take appropriate steps, such as adjusting the operation of the vacuum system 218 and/or alerting the user.

### E. Vacuum Applicators

FIGS. 3A-8B illustrate various embodiments of vacuum applicators suitable for use with the treatment systems described herein (e.g., treatment system 100 of FIGS. 1A-1C, treatment system 200 of FIG. 2A). The vacuum applicators of FIGS. 3A-8B can be fluidly connected to a vacuum system in order to apply suction to the patient's tissue. Additionally, the vacuum applicators can be fluidly connected to a cooling system (e.g., cooling system 105 of FIG. 1A, cooling system 212 of FIG. 2B) that circulates coolant in order to cool a patient's tissue.

FIGS. 3A-3I illustrate a vacuum applicator 300 (“applicator 300”) configured in accordance with embodiments of the present technology. Referring first to FIGS. 3A (top perspective view), 3B (top view) and 3C (side cross-sectional view) together, the applicator 300 has an elongated shape with a proximal end 301a, a distal end 301b, and a cup assembly 302 between the proximal and distal ends 301a, 301b. The cup assembly 302 can also have an elongated shape, with the longitudinal axis of the cup assembly 302 being aligned with the proximal-distal axis of the applicator 300. The cup assembly 302 can be used for cooling tissue and/or applying suction to tissue. The proximal end 301a of the applicator 300 can be configured to couple to a connector (e.g., connectors 104 of FIGS. 1A, 1C; connectors 204 of FIG. 2A) that provides coolant, vacuum, power, etc. to the cup assembly 302.

The applicator 300 also includes a housing 304 that supports and protects the cup assembly 302 and the internal components of the applicator 300. The housing 304 can be a waterproof housing, e.g., according to at least one of IPX1, IPX3, IPX4, or IPX7. The housing 304 can include an upper housing portion 305a and a lower or bottom housing portion 305b, and the cup assembly 302 can be mounted in the upper housing portion 305a. The upper housing portion 305a and lower housing portion 305b can be anti-condensation housings. In some embodiments, the housing 304 has a length within a range from 13.5 inches to 14.5 inches (e.g., 13.99 inches), a width within a range from 4 inches to 5 inches (e.g., 4.25 inches), and a height within a range from 4 inches to 5 inches (e.g., 4.67 inches). The total weight of the applicator 300 can be within a range from 2 lbs to 5 lbs (e.g., 3 lbs).

The cup assembly 302 can include a cup 306 and a contoured sealing element 308. The cup 306 can be contoured to define a tissue-receiving cavity 310 (“cavity 310”) with a concave heat-exchange surface 312 (“surface 312”). During operation of the applicator 300, a vacuum is applied to the patient's tissue to draw the tissue into the cavity 310 and into thermal communication with the surface 312. The cup 306 can be made partially or entirely of a thermally conductive material (e.g., a metal such as aluminum) to allow for efficient heat transfer to and/or from the patient's tissue. The cup 306 can also be in thermal communication with one or more thermal devices located within the housing 304, as described below.

To provide a suitable vacuum against the tissue, the sealing element 308 can extend along the perimeter or mouth of the cavity 310 and can sealingly engage, for example, a liner assembly, the patient's skin (e.g., if the applicator 300 is placed directly against skin), a cryoprotectant gel pad, or other surface. The sealing element 308 can be configured for forming airtight seals with the skin and can be made, in whole or in part, of silicon, rubber, soft plastic, or other suitable highly compliant materials. The mechanical properties, thermal properties, shape, and/or dimensions of the sealing element 308 can be selected based on, for example, whether it contacts the skin, a liner assembly, a cryoprotectant gel pad, or the like.

The shape of the cup 306 can be designed to conform to the patient's tissue to increase the volume of tissue that can be treated and improve treatment efficacy. For example, as can be seen in FIGS. 3A and 3B, the cup 306 can have a rounded, “banana-like” shape having a bottom 314 and spaced-apart sidewalls 316a, 316b. The bottom 314 and sidewalls 316a, 316b can be continually curved so that there are no “sharp” edges or corners within the cup 306; instead, the bottom 314 and sidewalls 316a, 316b are connected by smooth and gradual transitions. As shown in FIG. 3C, the cross-sectional surface profile of the cup 306 (e.g., along the longitudinal axis of the applicator 300) can have a curvature that corresponds to a higher order parabolic polynomial (e.g., 4th order or higher). The continually curved shape of the cup 306 can conform better to tissue (e.g., compared to cups having a more “U-like” shape with a flattened bottom and sidewalls), allow for large applicator sizes (e.g., so a larger tissue area can be treated), and provide a shallower cup curvature (e.g., to improve patient comfort). In some embodiments, the continually curved shape of the cup 306 allows tissue to be drawn into full contact against the surface 312 with few or no gaps or air pockets.

The dimensions of the cup 306 can be varied as desired. In some embodiments, for example, the width W₁ of the cup 306 (FIG. 3B) is within a range from 2 inches to 3 inches (e.g., 2.31 inches), the length L₁ of the cup 306 (FIG. 3C) is within a range from 9 inches to 10 inches (e.g., 9.54 inches), and the depth D₁ of the cup 306 (FIG. 3C) is within a range from 2 inches to 3 inches (e.g., 2.61 inches). The total treatment surface area (e.g., the area of surface 312) can be within a range from 30 square inches to 40 square inches (e.g., 34.9 square inches).

In some embodiments, the applicator 300 has a treatment area to weight ratio greater than or equal to 5 square inches/lb, 6 square inches/lb, 7 square inches/lb, 8 square inches/lb, 9 square inches/lb, 10 square inches/lb, 11 square inches/lb, 12 square inches/lb, 13 square inches/lb, 14 square inches/lb, 15 square inches/lb, 16 square inches/lb, 17 square inches/lb, 18 square inches/lb, 19 square inches/lb, or 20 square inches/lb. The applicator 300 can have a treatment area to tissue-draw depth ratio greater than or equal to 5 inches, 6 inches, 7 inches, 8 inches, 9 inches, 10 inches, 11 inches, 12 inches, 13 inches, 14 inches, 15 inches, 16 inches, 17 inches, 18 inches, 19 inches, or 20 inches. The tissue-draw depth of the cup 306 can be at least 50%, 75%, 80%, 85%, 90%, 95%, 99%, or 100% of the depth D₁.

The cup 306 can be configured to apply the vacuum to the patient's tissue via a vacuum port 318 (best seen in FIGS. 3B and 3C). The vacuum port 318 can be in fluid communication with the cavity 310. In the illustrated embodiment, the vacuum port 318 is positioned at the bottom 314 of the cavity 310 to comfortably draw the tissue deep into the cavity. Optionally, one or more vacuum grooves or air-egress features 320 (best seen in FIG. 3B) can be formed in the cup 306 near the vacuum port 318. The air-egress features 320 can help distribute the vacuum across the cup/tissue interface to enhance patient comfort, prevent air gaps (e.g., air gaps at the tissue/cup interface during tissue draw), and/or reduce vacuum leaks. After the subject's tissue fills the tissue-receiving cavity 310, the air-egress features 320 can continue to distribute the vacuum across a large area of the tissue-cup interface to keep the subject's tissue in the 310. During subcutaneous treatments, the subject's skin can extend across the air-egress features 320, illustrated as grooves or channels spreading outwardly from a central region of the cup 306. Constant or varying vacuum levels can be used to keep the tissue in thermal contact with the cup 306.

The air-egress features 320 can be grooves or channels that are machined into the surface 312 of the cup 306. For example, in the illustrate embodiment, the air-egress features 320 have a branching shape that extends from the vacuum port 318 along the bottom 314 and towards the sidewalls 316a, 316b. The number, positions, and geometries of the air-egress features 320 can be selected to define an airflow pattern suitable for evacuating air between the tissue and the cup 306. The air-egress features 320 also reduce the likelihood of air bubbles between the tissue and the cup 306. The air-egress features 320 can be positioned at locations at which air tends to become trapped. If ambient air is inadvertently sucked between the cup 306 and the subject's skin, it can serve as a thermal insulator and reduce heat transfer between the applicator 300 and the subject's tissue. Such air can be removed via the air-egress features 320 to maintain suitable thermal contact throughout the entire treatment session, including relatively long sessions (e.g., sessions equal to or longer than 20 minutes, 30 minutes, 45 minutes, 1 hour, or 2 hours). In some embodiments, the vacuum port 318 is positioned at central region of the cup 306 to draw the tissue into the deepest region of the tissue-receiving cavity 310, and the air-egress features 320 extend toward a peripheral portion of the surface 312. During cooling/heating, the tissue can fill substantially the entire cavity 310. In various embodiments, the air-egress features 320 can maintain airflow paths extending to the peripheral portion of the cup 306 such that the tissue occupies at least 80%, 90%, 92.5%, 95%, 99%, or 100% of the volume of the cavity 310. Accordingly, the subject's tissue can substantially fill an entire volume of the cavity 310. In one application, the subject's tissue fills 90% or more of the volume of the cavity 310.

In some embodiments, the surfaces of the applicator 300 (e.g., the exposed surfaces of the housing 304 and cup 306) have a smooth surface finish. For example, the roughness of the surfaces can be less than or equal to Ra 65, 60, 55, 50, 45, 40, 35, 32, or 30. In some embodiments, the surface 312 of the cup 306 has an Ra less than or equal to 32, and a backside of the cup 306 has an Ra less than or equal to 63. For example, most or substantially all of the surface 312 can have an average Ra less than or equal to 25, 30, or 35. Smooth surfaces can be produced, for example, by machining followed by an anodizing process. In some embodiments, the surface 312 can be a metal surface (e.g., an aluminum surface, a metal alloy surface, etc.) that is machined, polished, and/or anodized. A smoother surface can facilitate cleaning of the applicator 300, e.g., particularly the air-egress features 320.

FIG. 3D is a bottom perspective view of the applicator 300. As can be seen in FIGS. 3C and 3D together, the vacuum port 318 can be in fluid communication with a manifold 322 for receiving a gel trap (e.g., trap 165 of FIG. 1B—not shown in FIGS. 3C and 3D). The manifold 322 can be located beneath the cup 306. The bottom housing portion 305b can include an aperture 324 providing access to the manifold 322 for placement and removal of the gel trap. The gel trap can be configured to collect gel and/or other fluid that may be drawn into the vacuum port 318, as described in greater detail below. In some embodiments, the air-egress features 320 are also configured to facilitate flow of gel and/or other fluid into the gel trap.

Referring again to FIGS. 3A-3C together, the cup 306 can further include one or more sensors 326 on the surface 312 configured to monitor the patient's tissue during treatment. In some embodiments, the sensors 326 are temperature sensors (e.g., thermistors) that are configured to measure the temperature of the tissue. In other embodiments, the sensors 326 can include other types of sensors, such as pressure sensors, contact sensors, impedance sensors, and so on. The cup 306 can include any suitable number of sensors 326, such as one, two, three, four, five, six, seven, eight, nine, ten, or more sensors 326. The sensors 326 can be part of a flexible circuit that is embedded within the surface 312 of the cup 306. The sensor data generated by the sensors 326 can be transmitted to other components of the treatment system (e.g., circuit boards 210, applicator controllers 224 and/or main controller 222 of FIG. 2A) to monitor the treatment procedure and/or provide feedback for controlling the operation of the applicator 300.

FIGS. 3E-3I illustrate the applicator 300 at various stages during an assembly procedure. Referring first to FIG. 3E, which is an exploded view of the cup assembly 302 during a stage of the assembly procedure, the sealing element 308 can be attached to the edges of the cup 306 (e.g., via glue, sealant, or other adhesives; or by overmolding). The sensors 326 can be inserted into and secured within shallow recesses 328 formed in the sidewalls 316a, 316b of the cup 306. The recesses 328 can prevent the edges of the sensors 326 from being caught and peeled off during cleaning of the cup 306. Additionally, the configuration of the sensors 326 and recesses 328 can allow the sidewalls 316a, 316b to be continuously curved.

Referring next to FIG. 3F, which is a bottom perspective view of the cup assembly 302 during another stage of the assembly procedure, a first thermal device 330a and a second thermal device 330b (collectively, “thermal devices 330”) can be mounted to the bottom surface of the cup 306. The thermal devices 330 can be positioned on opposite sides of the cup 306 and can be oriented generally along the longitudinal axis of the cup 306. Each thermal device 330 can include one or more thermoelectric elements 332 for cooling/heating the cup 306. For example, the thermoelectric elements 332 can be thermoelectric coolers (TECs). The TECs can be configured to operate in both a cooling mode and a heating mode. The thermoelectric elements 332 can be coupled to the bottom surface of the cup 306 (e.g., either directly or indirectly via thermal pads or other thermally conductive materials). As previously described, the cup 306 can be made of a thermally conductive material so that the cooling/heating applied by the thermoelectric elements 332 is transferred via the cup 306 to the patient's tissue. Optionally, each thermal device 330 can include one or more temperature sensors 333 (e.g., thermistors) for monitoring the temperature of the thermoelectric elements 332. The temperature sensors 333 can be separate from the temperature sensors 326 located on the surface 312 of the cup 306. For example, a thermistor can be located between each thermoelectric element 332 and the bottom surface of the cup 306.

In the illustrated embodiment, each thermal device 330 has three thermoelectric elements 332 such that the applicator 300 includes a total of six thermoelectric elements 332 corresponding to six cooling/heating zones. In other embodiments, each thermal device 330 can have a different number of the thermoelectric elements 332 (e.g., one, two, four, five, or more) and cooling/heating zones. Additionally, the sizes of the thermoelectric elements 332 can be varied as desired to provide different cooling/heating capabilities. For example, each thermoelectric element 332 can be approximately 30 mm by 40 mm in size. The thermoelectric elements 332 can be addressable thermoelectric elements that are each independently controllable (e.g., by a remote applicator controller, as discussed in greater detail below).

Each thermal device 330 can also include a fluid-cooled element 334 attached to the backside of the thermoelectric elements 332 for cooling/heating the thermoelectric elements 332. In a cooling mode, the fluid-cooled element 334 can cool the backside of the thermoelectric elements 332 to keep the thermoelectric elements 332 at or below a target temperature. In a heating mode, the fluid-cooled element 334 can heat the backside of the thermoelectric elements 332 to keep the thermoelectric elements 332 at or above a target temperature. The fluid-cooled element 334 can include internal fluid channels or passages (not shown) and ports 335 for circulation of a coolant from a cooling system (e.g., cooling system 212 of FIG. 2B). The total weight of the applicator 300 can increase less than 1%, 2%, 3%, 4%, or 5% when filled with fluid coolant (e.g., water) to reduce the occurrence of pop offs due to coolant flows, changes in coolant flow, etc.

FIG. 3G is a bottom perspective view of the cup assembly 302 during another stage of the assembly procedure. Referring to FIGS. 3F and 3G together, an insulating material 336 (e.g., foam) can be positioned over the bottom surface of the cup 306 and the backsides of the thermal devices 330. The gel trap manifold 322 can be attached to the bottom surface of the cup 306 near the vacuum port 318. A bypass tube 337 can be used to fluidly couple the fluid-cooled elements 334.

A first circuit board 338a and a second circuit board 338b (collectively, “circuit boards 338”) can be electrically coupled to the thermoelectric elements 332, the sensors 326, the sensors 333, and/or other electronic components of the applicator 300. Optionally, the circuit boards 338 can be electrically coupled to each other via a cable 340 or other electrical connector. The circuit boards 338 may be identical or generally similar to the circuit boards 210 of FIG. 2A. The circuit boards 338 can be configured to obtain data (e.g., voltage data, current data, etc.) from the thermoelectric elements 332, the sensors 326, the sensors 333, and/or other electronic components of the applicator 300. In some embodiments, the circuit boards 338 perform little or no processing of the data. Instead, the circuit boards 338 can simply transmit the data to a component remote from the applicator 300, such as a control unit (e.g., control unit 206 of FIG. 2A). Additionally, the circuit boards 338 can route control and/or power signals generated by a control unit or other remote component to the corresponding applicator components (e.g., thermoelectric elements 332, sensors 326, sensors 333, and/or other electronic components).

Optionally, each circuit board 338 can include a contamination circuit configured to detect the presence and/or ingress of fluid. For example, fluids such as water (e.g., from drip condensation) or coolant (e.g., due to leaks) may be present in the applicator 300 during operation. Fluid ingress may be caused by submerging the applicator 300 in liquid for extended periods of time. Fluid accumulation near thermistors can adversely affect temperature measurements. Fluid can also cause electrical shorts and/or damage the internal components of the applicator 300. Accordingly, the contamination circuit can be used to detect whether fluid has entered the applicator 300, and, if so, shut down operation of the applicator 300. For example, the contamination circuit can initially be in an open state, and can switch to a closed state if water enters the applicator 300. For example, the contamination circuit can include one or more water detectors. FIG. 3G-1 shows a water detector in the form of an open switch 339. When water contacts the switch 339, the switch is closed indicating the presence of water (e.g., freestanding liquid capable of contacting circuitry within the applicator 300). A controller in communication with the switch 339 can be programmed to identify detection of moisture based on one or more signals from the switch 339. The number, positions, and configurations of water detectors can be selected based on the configuration of the circuit board 338, locations susceptible to condensation, location of electrical components, etc. In some embodiments, water detectors are positioned proximate to or on anti-condensation housings, integrated into circuit boards, coupled to exposed cooled metal surfaces inside the applicator 300, or the like.

The limited functionality of the circuit boards 338 can provide various benefits, such as reducing the thermal footprint of the applicator 300—excess heat can increase the load on the thermoelectric elements 332, create condensation that may adversely affect electronic components within the applicator 300, create safety issues (e.g., overheating), and reduce treatment efficacy. This approach can also reduce the electrical load for operating the applicator 300, and thus the amount and size of the wiring, which can allow for a more flexible connector cable with detachable bayonet connections, as described in detail below. For example, the wiring used in the applicator 300 can be less than or equal to 20 AWG, or less than or equal to 28 AWG. Additionally, the size, weight, and cost of the applicator 300 can be reduced. A lighter applicator 300 can be more comfortable for the patient, easier to secure to the patient's body (e.g., via straps or adhesive coupling gel), and less likely to pop off during operation.

FIGS. 3H and 3I are a bottom view and exploded view, respectively, of the applicator 300 during another stage of the assembly procedure. Referring to FIGS. 3H and 3I together, the cup assembly 302 and associated components can be positioned within and attached to the upper housing portion 305a. A supply fluid line 342a and a return fluid line 342b can be fluidly coupled to the fluid-cooled elements 334 (not shown) so that coolant can circulate through the fluid-cooled elements 334 (e.g., as indicated by arrows in FIG. 3H). In the illustrated embodiment, the fluid supply and return lines 342a, 342b are located at or near the proximal end 301a of the applicator 300 while the bypass tube 337 is located at or near the distal end 301b.

The supply fluid line 342a and return fluid line 342b can be coupled to an interconnect assembly 344 at the distal end 301b of the applicator 300. The interconnect assembly 344 can also include interfaces 346 for receiving a vacuum line (not shown) connected to the gel trap manifold 322 (e.g., via hose barb 348), and one or more electrical lines (not shown) connected to the circuit boards 338. As described in greater detail below, the assembly receptacle 344 can include features for releasably coupling the applicator 300 to a connector (e.g., connectors 104 of FIGS. 1A, 1C; connectors 204 of FIG. 2A). This approach allows the applicator 300 to be separated from the connector, e.g., for more convenient cleaning and/or storage.

As shown in FIG. 3I, the bottom housing portion 305b can be attached to the upper housing portion 305a to enclose the internal components of the applicator 300. The upper housing portion 305a and bottom housing portion 305b can be configured to form a water-tight seal. This approach allows the applicator 300 to be partially or fully submerged without fluid entering the interior of the applicator 300, which may allow for more simpler, easier, and more effective cleaning procedures.

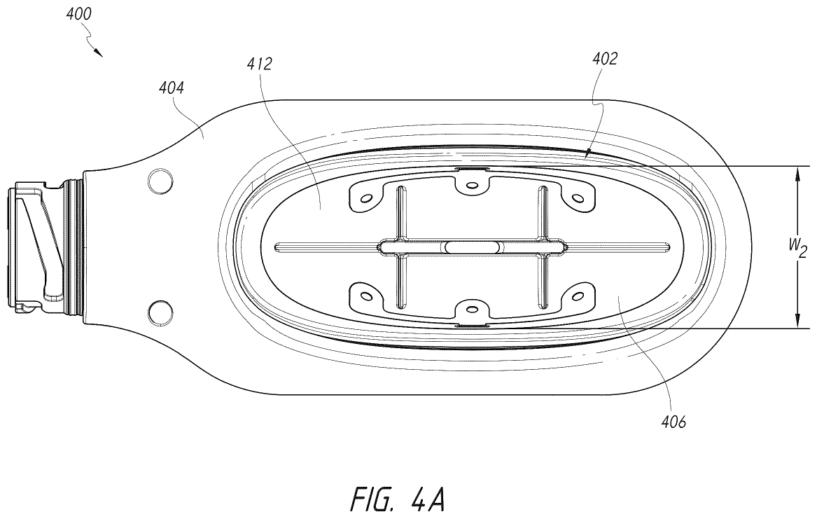

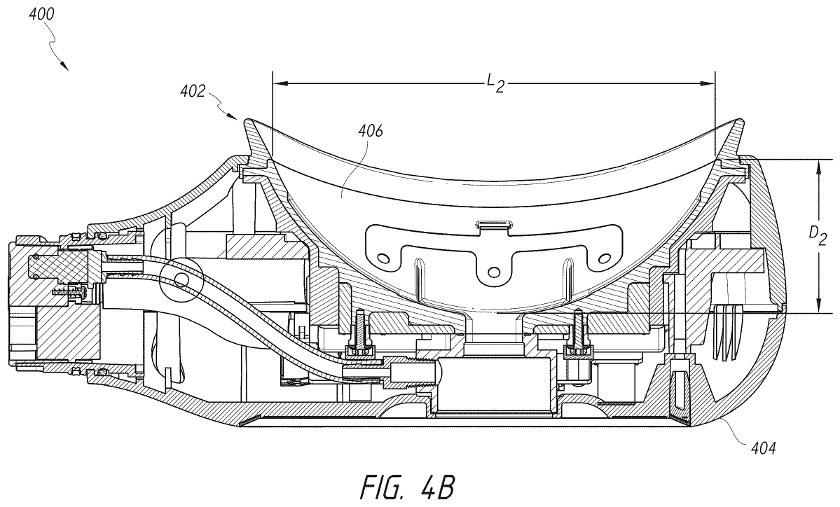

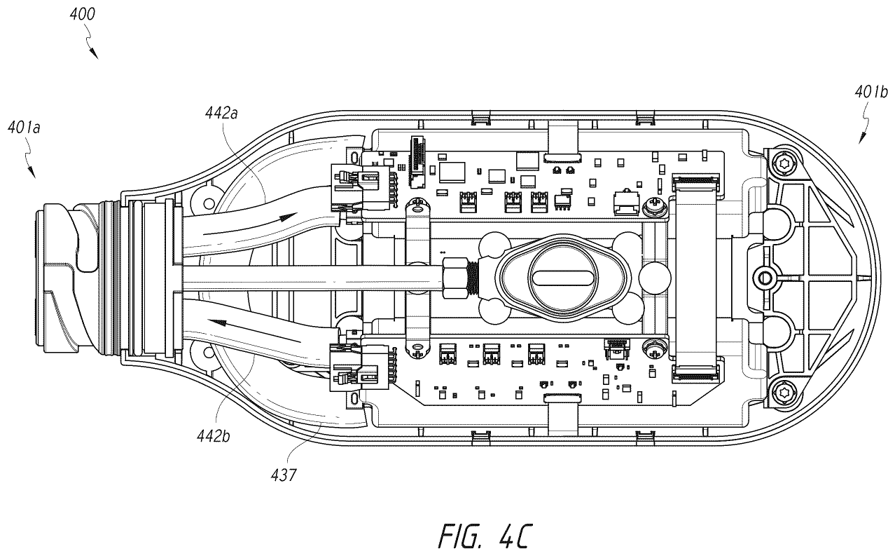

FIGS. 4A-8B illustrate vacuum applicators configured in accordance with additional embodiments of the present technology. The features of the applicators described with respect to FIGS. 4A-8B may be generally similar to the features of the applicator 300 of the applicator 300 of FIGS. 3A-3I, such that like reference numbers indicate identical or similar elements (e.g., cup assembly 302 versus cup assembly 402). Accordingly, the discussion of the applicators illustrated in FIGS. 4A-8B will be limited to those features that differ from the applicator 300 of FIGS. 3A-3I.

FIGS. 4A-4C illustrate a vacuum applicator 400 (“applicator 400”) configured in accordance with embodiments of the present technology. Referring first to FIGS. 4A (top view) and 4B (side cross-sectional view) together, the applicator 400 includes a cup assembly 402 for cooling tissue, and a housing 404 supporting and protecting the cup assembly 402. The applicator 400 can be designed to treat a smaller tissue area than the applicator 300 of FIGS. 3A-3I. In some embodiments, for example, the housing 404 has a length within a range from 10 inches to 11 inches (e.g., 10.53 inches), a width within a range from 3.5 inches to 4.5 inches (e.g., 4.17 inches), and a height within a range from 3.5 inches to 4.5 inches (e.g., 4.17 inches). The total weight of the applicator 400 can be within a range from 2 lbs to 3 lbs (e.g., 2.4 lbs).

The cup assembly 402 can include a cup 406 having a rounded, continually curved shape. In some embodiments, the width W₂ of the cup 406 (FIG. 4A) is within a range from 2 inches to 3 inches (e.g., 2.31 inches), the length L₂ of the cup 406 (FIG. 4B) is within a range from 5.5 inches to 6.5 inches (e.g., 5.99 inches), and the depth D₂ of the cup 406 (FIG. 4B) is within a range from 1.5 inches to 2.5 inches (e.g., 2.07 inches). The total treatment surface area (e.g., the area of surface 412) can be within a range from 15 square inches to 25 square inches (e.g., 20.6 square inches). Due to the smaller surface area of the cup 406, the air-egress features 420 can also be smaller and include fewer branches than the air-egress features 320 of the applicator 300.

In some embodiments, the applicator 400 has a treatment area to weight ratio greater than or equal to 5 square inches/lb, 6 square inches/lb, 7 square inches/lb, 8 square inches/lb, 9 square inches/lb, 10 square inches/lb, 11 square inches/lb, 12 square inches/lb, 13 square inches/lb, 14 square inches/lb, 15 square inches/lb, 16 square inches/lb, 17 square inches/lb, 18 square inches/lb, 19 square inches/lb, or 20 square inches/lb. The applicator 400 can have a treatment area to tissue-draw depth ratio greater than or equal to 5 inches, 6 inches, 7 inches, 8 inches, 9 inches, 10 inches, 11 inches, 12 inches, 13 inches, 14 inches, 15 inches, 16 inches, 17 inches, 18 inches, 19 inches, or 20 inches. The tissue-draw depth of the cup 406 can be at least 50%, 75%, 80%, 85%, 90%, 95%, 99%, or 100% of the depth D₂.

FIG. 4C is a bottom view of the applicator 400 during an assembly procedure. The configuration of the internal components of the applicator 400 may be identical or generally similar to the applicator 300, except that the bypass tube 437 for the fluid-cooled elements (not shown) of the applicator 400 is located at the proximal end 401a of the applicator 400 along with the fluid supply and return lines 442a, 442b, rather than at the distal end 401b.

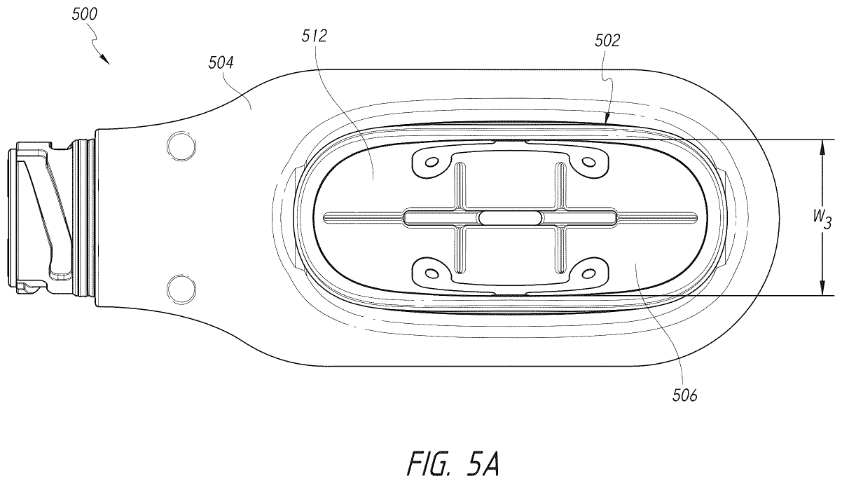

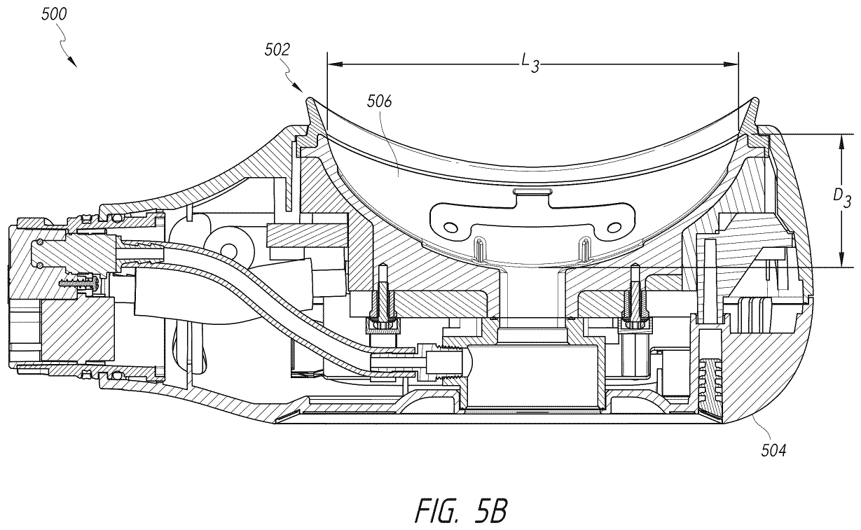

FIGS. 5A and 5B are top and side cross-sectional views, respectively, of a vacuum applicator 500 (“applicator 500”) configured in accordance with embodiments of the present technology. The applicator 500 includes a cup assembly 502 for cooling tissue, and a housing 504 supporting and protecting the cup assembly 502. The applicator 500 can be designed to treat a smaller tissue area than the applicators 300, 400 of FIGS. 3A-4C. In some embodiments, for example, the housing 504 has a length within a range from 9 inches to 10 inches (e.g., 9.43 inches), a width within a range from 3 inches to 4 inches (e.g., 3.62 inches), and a height within a range from 3 inches to 4 inches (e.g., 3.62 inches). The total weight of the applicator 500 can be within a range from 1 lb to 2 lbs (e.g., 1.7 lbs).

The cup assembly 502 can include a cup 506 having a rounded, continually curved shape. In some embodiments, the width W₃ of the cup 506 (FIG. 5A) is within a range from 1.5 inches to 2.5 inches (e.g., 1.90 inches), the length L₃ of the cup 506 (FIG. 5B) is within a range from 4.5 inches to 5.5 inches (e.g., 4.80 inches), and the depth D₃ of the cup 506 (FIG. 5B) is within a range from 1 inch to 2 inches (e.g., 1.51 inches). The total treatment surface area (e.g., the area of surface 512) can be within a range from 8 square inches to 18 square inches (e.g., 13 square inches).

In some embodiments, the applicator 500 has a treatment area to weight ratio greater than or equal to 5 square inches/lb, 6 square inches/lb, 7 square inches/lb, 8 square inches/lb, 9 square inches/lb, 10 square inches/lb, 11 square inches/lb, 12 square inches/lb, 13 square inches/lb, 14 square inches/lb, 15 square inches/lb, 16 square inches/lb, 17 square inches/lb, 18 square inches/lb, 19 square inches/lb, or 20 square inches/lb. The applicator 500 can have a treatment area to tissue-draw depth ratio greater than or equal to 5 inches, 6 inches, 7 inches, 8 inches, 9 inches, 10 inches, 11 inches, 12 inches, 13 inches, 14 inches, 15 inches, 16 inches, 17 inches, 18 inches, 19 inches, or 20 inches. The tissue-draw depth of the cup 506 can be at least 50%, 75%, 80%, 85%, 90%, 95%, 99%, or 100% of the depth D₃.

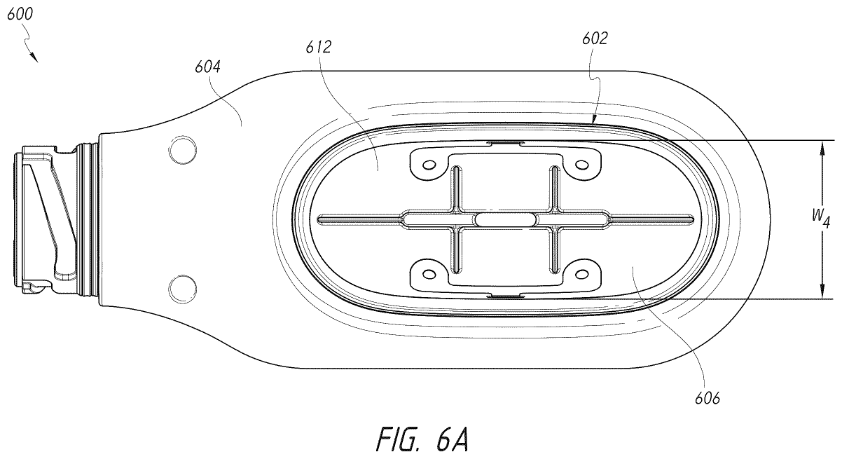

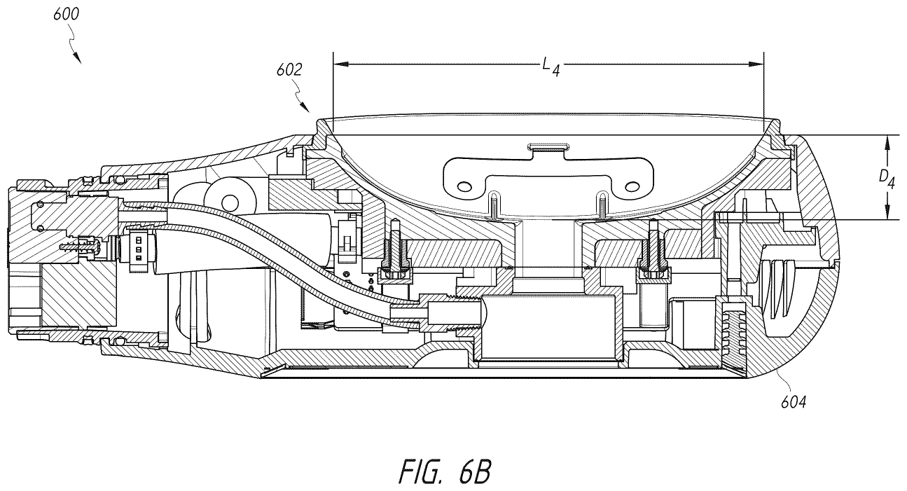

FIGS. 6A and 6B are top and side cross-sectional views, respectively, of a vacuum applicator 600 (“applicator 600”) configured in accordance with embodiments of the present technology. The applicator 600 includes a cup assembly 602 for cooling tissue, and a housing 604 supporting and protecting the cup assembly 602. In some embodiments, the housing 604 has a length within a range from 9 inches to 10 inches (e.g., 9.51 inches), a width within a range from 3 inches to 4 inches (e.g., 3.74 inches), and a height within a range from 2.5 inches to 3.5 inches (e.g., 3.03 inches). The total weight of the applicator 600 can be within a range from 1 lb to 2 lbs (e.g., 1.6 lbs).

The cup assembly 602 can include a cup 606 having a rounded, continually curved shape. As best seen in FIG. 6B, the cup 606 can have a more shallow, flattened shape compared to the applicators 300, 400, 500 of FIGS. 3A-5B. In some embodiments, for example, the width W₄ of the cup 606 (FIG. 6A) is within a range from 1.5 inches to 2.5 inches (e.g., 2 inches), the length La of the cup 606 (FIG. 6B) is within a range from 4.5 inches to 5.5 inches (e.g., 4.92 inches), and the depth D₄ of the cup 606 (FIG. 6B) is within a range from 0.5 inches to 1.5 inches (e.g., 1 inch). The total treatment surface area (e.g., the area of surface 612) can be within a range from 8 square inches to 18 square inches (e.g., 12.9 square inches).

In some embodiments, the applicator 600 has a treatment area to weight ratio greater than or equal to 5 square inches/lb, 6 square inches/lb, 7 square inches/lb, 8 square inches/lb, 9 square inches/lb, 10 square inches/lb, 11 square inches/lb, 12 square inches/lb, 13 square inches/lb, 14 square inches/lb, 15 square inches/lb, 16 square inches/lb, 17 square inches/lb, 18 square inches/lb, 19 square inches/lb, or 20 square inches/lb. The applicator 600 can have a treatment area to tissue-draw depth ratio greater than or equal to 5 inches, 6 inches, 7 inches, 8 inches, 9 inches, 10 inches, 11 inches, 12 inches, 13 inches, 14 inches, 15 inches, 16 inches, 17 inches, 18 inches, 19 inches, or 20 inches. The tissue-draw depth of the cup 606 can be at least 50%, 75%, 80%, 85%, 90%, 95%, 99%, or 100% of the depth D₄.

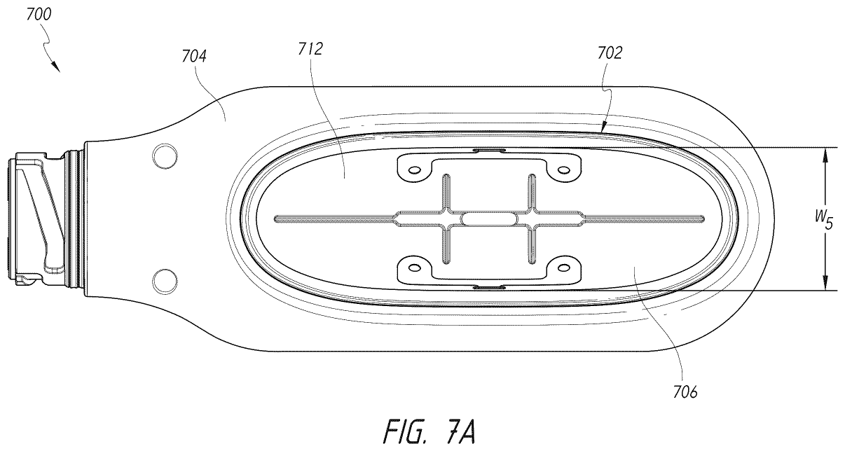

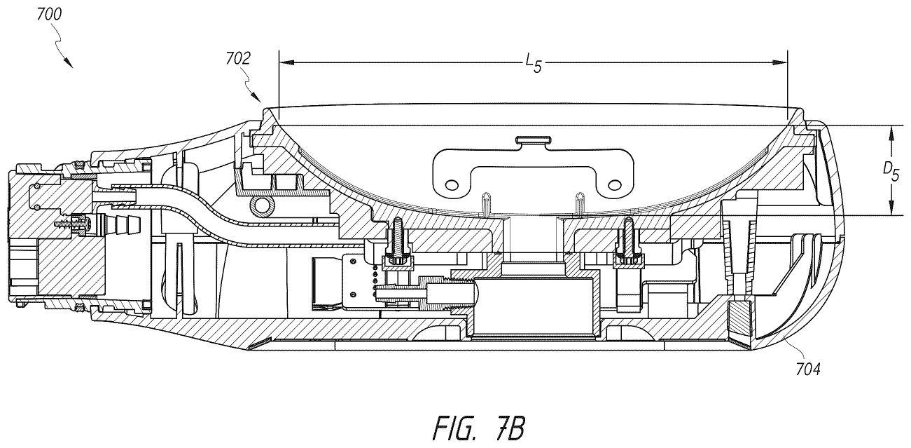

FIGS. 7A and 7B are top and side cross-sectional views, respectively of a vacuum applicator 700 (“applicator 700”) configured in accordance with embodiments of the present technology. The applicator 700 can be generally similar to applicator 600 of FIGS. 6A and 6B, except that the applicator 700 is designed to treat a larger tissue area than the applicator 600. The applicator 700 includes a cup assembly 702 for cooling tissue, and a housing 704 supporting and protecting the cup assembly 702. In some embodiments, the housing 704 has a length within a range from 10 inches to 11 inches (e.g., 10.65 inches), a width within a range from 3 inches to 4 inches (e.g., 3.70 inches), and a height within a range from 2.5 inches to 3.5 inches (e.g., 3.15 inches). The total weight of the applicator 700 can be within a range from 1 lb to 2 lbs (e.g., 1.6 lbs).

The cup assembly 702 can include a cup 706 having a rounded, continually curved shape. The cup 706 can have a more shallow, flattened shape compared to the applicators 300, 400, 500 of FIGS. 3A-5B. In some embodiments, the width W₅ of the cup 706 (FIG. 7A) is within a range from 1.5 inches to 2.5 inches (e.g., 2 inches), the length L₅ of the cup 706 (FIG. 7B) is within a range from 6 inches to 7 inches (e.g., 6.5 inches), and the depth D₅ of the cup 706 (FIG. 7B) is within a range from 0.5 inches to 1.5 inches (e.g., 1.13 inches). The total treatment surface area (e.g., the area of surface 712) can be within a range from 10 square inches to 20 square inches (e.g., 14.9 square inches).

In some embodiments, the applicator 700 has a treatment area to weight ratio greater than or equal to 5 square inches/lb, 6 square inches/lb, 7 square inches/lb, 8 square inches/lb, 9 square inches/lb, 10 square inches/lb, 11 square inches/lb, 12 square inches/lb, 13 square inches/lb, 14 square inches/lb, 15 square inches/lb, 16 square inches/lb, 17 square inches/lb, 18 square inches/lb, 19 square inches/lb, or 20 square inches/lb. The applicator 700 can have a treatment area to tissue-draw depth ratio greater than or equal to 5 inches, 6 inches, 7 inches, 8 inches, 9 inches, 10 inches, 11 inches, 12 inches, 13 inches, 14 inches, 15 inches, 16 inches, 17 inches, 18 inches, 19 inches, or 20 inches. The tissue-draw depth of the cup 706 can be at least 50%, 75%, 80%, 85%, 90%, 95%, 99%, or 100% of the depth D₅.

FIGS. 8A and 8B illustrate a vacuum applicator 800 (“applicator 800”) configured in accordance with embodiments of the present technology. The applicator 800 has an elongated shape with a distal end 801a, a proximal end 801b, and a cup assembly 802 for cooling tissue between the distal and proximal ends 801a, 801b. The cup assembly 802 can also have an elongated shape, with the longitudinal axis of the cup assembly 802 being orthogonal to the proximal-distal axis of the applicator 300. In some embodiments, the proximal end 801b is integrally formed with or fixedly coupled to a connector (e.g., connectors 104 of FIGS. 1A, 1C; connectors 204 of FIG. 2A) that provides coolant, vacuum, power, etc. to the cup assembly 802. In other embodiments, however, the proximal end 801b can be removably coupled to the connector.

The applicator 800 also includes a housing 804 supporting and protecting the cup assembly 802. In some embodiments, the housing 804 has a length within a range from 3.5 inches to 4.5 inches (e.g., 4.09 inches), a width within a range from 2 inches to 3 inches (e.g., 2.31 inches), and a height within a range from 4 inches to 5 inches (e.g., 4.36 inches). The total weight of the applicator 800 can be within a range from 0.5 lbs to 1.5 lbs (e.g., 0.9 lbs).

The cup assembly 802 can include a cup 806 having a rounded, continually curved shape. The cup 806 can be designed to treat a relatively small tissue area (e.g., a submental area). In some embodiments, for example, the width W₆ of the cup 806 (FIG. 8A) is within a range from 0.5 inches to 1.5 inches (e.g., 1.06 inches), the length Le of the cup 806 (FIG. 8A) is within a range from 2.5 inches to 3.5 inches (e.g., 3.15 inches), and the depth De of the cup 806 (FIG. 8B) is within a range from 0.5 inches to 1.5 inches (e.g., 1.10 inches). The total treatment surface area (e.g., the area of surface 812) can be within a range from 2 square inches to 8 square inches (e.g., 5.4 square inches).

In some embodiments, the applicator 800 has a treatment area to weight ratio greater than or equal to 1 squares inches/lb, 2 square inches/lb, 3 square inches/lb, 4 square inches/lb, 5 square inches/lb, 6 square inches/lb, 7 square inches/lb, 8 square inches/lb, 9 square inches/lb, 10 square inches/lb, 11 square inches/lb, 12 square inches/lb, 13 square inches/lb, 14 square inches/lb, 15 square inches/lb, 16 square inches/lb, 17 square inches/lb, 18 square inches/lb, 19 square inches/lb, or 20 square inches/lb. The applicator 800 can have a treatment area to tissue-draw depth ratio greater than or equal to 1 inch, 2 inches, 3 inches, 4 inches, 5 inches, 6 inches, 7 inches, 8 inches, 9 inches, 10 inches, 11 inches, 12 inches, 13 inches, 14 inches, 15 inches, 16 inches, 17 inches, 18 inches, 19 inches, or 20 inches. The tissue-draw depth of the cup 806 can be at least 50%, 75%, 80%, 85%, 90%, 95%, 99%, or 100% of the depth D₆.

The applicator 800 can include a cavity 850 for receiving a gel trap 852. The cavity 850 can be formed in the bottom 814 of the cup 806 so that a portion of the gel trap 852 is exposed. The gel trap 852 can be configured to collect gel or other fluid that may be drawn into the vacuum port 818, as described in greater detail below.

### F. Vacuum Trap

FIG. 9A is an isometric view of an applicator 900 with a gel trap 910 configured in accordance with embodiments of the present technology. FIG. 9B is a side cross-sectional view of the applicator 900 of FIG. 9A. The gel trap 910 can capture gel (e.g., cryoprotectant gel, adhesive gel, etc.), liquid (e.g., water associated with condensation), and other substances. The gel trap 910 can include a cap 912 with opposing ends 916, 918, illustrated overlying respective recessed regions 917, 919, that can be gripped to pull the gel trap 910 from the applicator 900. After emptying the gel trap 910, the emptied gel trap 910 can be reinserted into the applicator 900. The gel trap 910 can be used with different applicators, including the applicators discussed in connection with FIGS. 1A and 3A-7B. A treatment system can include a set of universal traps to enable batch cleaning of the traps without system downtime.

FIG. 9B shows the gel trap 910 installed in a manifold 944 accessible at a backside 906 of the applicator 900. The backside position of the gel trap 910 enhances treatment because an entire cup cooling surface 907 can effectively treat targeted tissue independent of the amount of gel trapped in the applicator 900. Additionally, the applicator 900 can include a cup 908 with a relatively large number of air-egress features 911, which can result in enhanced tissue draw and adhesion to the subject, especially at the perimeter of the cup 908. For example, the backside gel trap position allows for a more extensive air-egress feature network for limiting or reducing air pockets or bubbles (e.g., air pockets or bubbles at the bottom of the cup 908), which can significantly reduce heat transfer and adversely affect efficacy. In some embodiments, air-egress features disclosed in U.S. Patent Publication No. 2018/0310950 may be used with applicators and gel traps disclosed herein. The number of air-egress features in the networks of U.S. Patent Publication No. 2018/0310950 can be increased due to the backside position of the gel trap 910. In some embodiments, the air-egress features 911 are shallow grooves extending from a vacuum port 929 and have a depth of about 0.5 mm to about 2 mm, a width of about 1 mm to about 2 mm, and a length of at least about 5 mm, about 8 mm, about 10 mm, or about 15 mm and can have generally U-shaped, V-shaped, or other suitable cross-sectional shape. For example, the air-egress features 911 are channels each having a generally uniform cross-sectional U-shaped profile along its longitudinal length. In other embodiments, the depth and/or width can decrease in the direction away from the vacuum port 929. The dimensions, configuration, and characteristics of the air-egress features 911 can be selected based on desired airflow rates, position of air-egress features, number and/or sizes of the vacuum ports, or the like.

When a vacuum is drawn, the subject's skin can be held against substantially all of the cooling surface 907 at the bottom cavity 921. The region of the cooling surface 907 surrounding and adjacent the vacuum port 929 can be generally flat or slightly curved to help maintain thermal contact with the subject's skin. An operator can also view the gel trap 910 to confirm proper installation and can visually inspect the gel trap 910 at any time during treatment. A reservoir or chamber of the gel trap 910 is remote from the cooling surface 907. Captured gel 169 is held away from heat flow paths between the cooling surface 907 and the subject's tissue such that the amount of captured gel 169 does not affect cooling/heating of the target tissue to avoid interfering with treatment.

The gel trap 910 can be configured for toolless installation and/or toolless removal from the applicator 900. In installation, the gel trap 910 can establish a fluid tight connection with the manifold upon manual insertion. After treatment, the gel trap 910 can be removed, emptied, and reinstalled without using any tools. If the gel trap 910 is completely filled during a treatment session, the vacuum can be stopped and the gel trap 910 emptied. The applicator 900 can be held stationary against the subject while emptying the gel trap 910 to maintain proper applicator position. After installing a new gel trap or reinstalling the emptied gel trap 910, the vacuum can be restarted to resume treatment. In some embodiments, the applicator 900 can include a bypass line between the vacuum port 929 and the vacuum line 966. The bypass line can include one or more valves, hoses, fittings, or the like. When the gel trap 910 is removed from the applicator 900, the bypass line can be opened to maintain the vacuum. Gel traps can be replaced any number of times during a treatment without affecting tissue retention.

FIG. 9C is a detailed view of a portion of the applicator 900 of FIG. 9B. FIG. 9D is a cross-sectional view taken along line 9D-9D of FIG. 9C. Referring now to FIG. 9C, the gel trap 910 can sealingly engage the manifold 944 to provide fluid communication between the vacuum port 929 and an internal vacuum line 966 of the applicator 900. Referring now to FIG. 9D, the gel trap 910 can include a container 922 having an inlet 924, an outlet 926, and a reservoir or chamber 928 (“chamber 928”). The inlet 924 can be in fluid communication with a vacuum port 929, and the outlet 926 can be in fluid communication with a vacuum line (e.g., vacuum line 966 of FIG. 9C). The container 922 can include an air-permeable, gel-impermeable element or membrane 967 (“membrane 967”) extending across the outlet 926. Air can flow through the membrane 967, while the membrane 967 blocks the flow of gel, thereby capturing gel in the chamber 928.

The holding capacity of the chamber 928 can be greater than the volume of gel/liquid expected to be drawn into the applicator 900, volume of gel used in a procedure, etc. For example, a ratio of the volume of the chamber 928 to the volume of applied gel (e.g., gel present at the skin-applicator interface at the start of the procedure) can be 1, 1.5, 2, 2.5, 3, 3.5, 4, 4.5, or 5 and the chamber 928 can have a holding capacity equal to or greater than about 10 cm³, 15 cm³, 20 cm³, 25 cm³, 30 cm³, 35 cm³, 40 cm³, 45 cm³, 50 cm³, 55 cm³, 60 cm³, 65 cm³, 70 cm³, 75 cm³, 80 cm³, 85 cm³, 90 cm³, 95 cm³, 100 cm³, 200 cm³, 300 cm³, or another suitable volume. For example, a gel trap with a holding capacity of 60 cm³ can be used to perform three treatment sessions each using less than about 20 cm³ of gel. The configuration and holding capacity of the gel trap 910 can be selected based on the procedure to be performed. Optionally, the gel trap 910 can be sized such that it will not completely fill during any single treatment session.

FIGS. 9C and 9D show the gel trap 910 with a mouth 960 carrying a sealing member 962 compressed against an inner surface 963 of the manifold 944. The gel trap 910 can also include a sealing member 965 configured to be compressed against a sidewall 969 of the manifold 944. In some embodiments, both sealing members 962, 965 can form airtight seals with the manifold 944 to maintain a vacuum level sufficiently high to hold tissue in the applicator 900. Air can flow along a passageway 964 (FIG. 9D) between the container 922 and the manifold 944 and can exit the manifold 944 via the vacuum line 966 (FIG. 9C). Details of features of the gel trap 910 are discussed in connection with FIGS. 9E-9G.

FIG. 9E shows the container 922 including the cap 912 and a main body 968 having sections 970, 972 coupled together using one or more adhesives, welding, or the like. In other embodiments, the main body 968 has a one-piece construction formed via a machining or molding process. The multi-piece or one-piece main body 968 can also include one or more viewing windows made, in whole or in part, of a transparent material for viewing inside the container 922. This allows an operator to determine when the gel trap 910 should be emptied. For example, the cap 912 can include a viewing window.

FIG. 9F is an exploded isometric view of the gel trap 910. The container 922 has recesses 972, 976 configured to hold the sealing members 962, 965, respectively. The outlet 926, illustrated as a generally rectangular window or opening, is positioned between the recesses 972, 976. The recesses 972, 976 can be U-shaped recesses, V-shaped recesses, or other features shaped to hold the respective sealing members 962, 965. The sealing members 962, 965 can be gaskets, O-rings, or other sealing elements made, in whole or in part, of a compressible material (e.g., rubber, silicon, polymers, etc.) to form a seal (e.g., an airtight seal, a watertight seal, etc.) that inhibits or prevents leakage between the gel trap 910 and the applicator. In some embodiments, airtight seals provided by the sealing members 962, 965 can be maintained under vacuum pressure of at least 2 inHg, 3 inHg, 4 inHg, 5 inHg, 6 inHg, 7 inHg, 8 inHg, 9 inHg, 10 inHg, or 12 inHg. For example, substantially airtight seals can be maintained when the vacuum level is between 6 inHg and 10 inHg and varied at a rate of 0.1 inHg/second, 0.5 inHg/second, 1 inHg/second, or 2 inHg/second. The vacuum can be maintained during periodic air leakage between the applicator the patient skin (e.g., air leakage caused by excessive body movement of the user). In some embodiments, vacuum leak rates at the gel trap 910 and manifold 944 interface can be equal to or less than about 0.05 LPM, 0.1 LPM, 0.2 LPM, 0.5 LPM, 1 LPM, 1.5 LPM, or 2 LPM at the pressure levels disclosed herein. For example, a vacuum leak rate at each seal or both seals can be equal to or less than about 0.05 LPM at 8 inHg, 0.1 LPM at 8 inHg, 0.25 LPM at 8 inHg, 0.5 LPM at 8 inHg, or 1 LPM at 8 inHg. The configuration of the gel trap 910 and manifold 944 can be selected based on the desired vacuum levels, maximum air leakage rates, and other operating parameters.

FIG. 9G is an exploded view of the air-permeable membrane 967 and section 970. The air-permeable membrane 967 can include one or more gel-impermeable membranes, filters, valves, or other elements for selectively blocking liquids/gels (or other substances) while maintaining air flow therethrough. The air-permeable membrane 967 can be an air-permeable and liquid/gel-impermeable membrane that extends across the entire outlet 926. The air-permeable membrane 967 can block the flow of gel or other liquid such that at least 90%, 95%, 97%, 98%, or 99% of the total weight or volume of the captured gel or liquid is kept in the gel trap 910 at air flow rates and pressure levels disclosed herein for at least 1 minute, 2 minutes, 10 minutes, 30 minutes, or 1 hour. The configuration, size, and characteristics of the membrane 967 can be selected based on the characteristics of gel(s) used during the procedure, or the like.

### G. Non-Vacuum Applicators

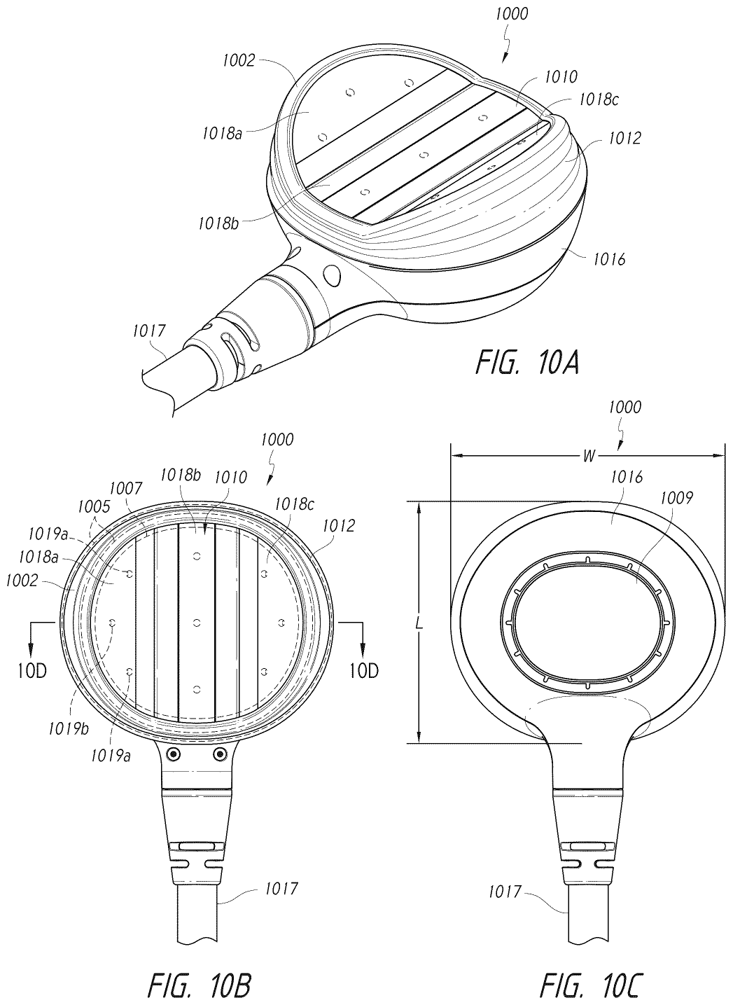

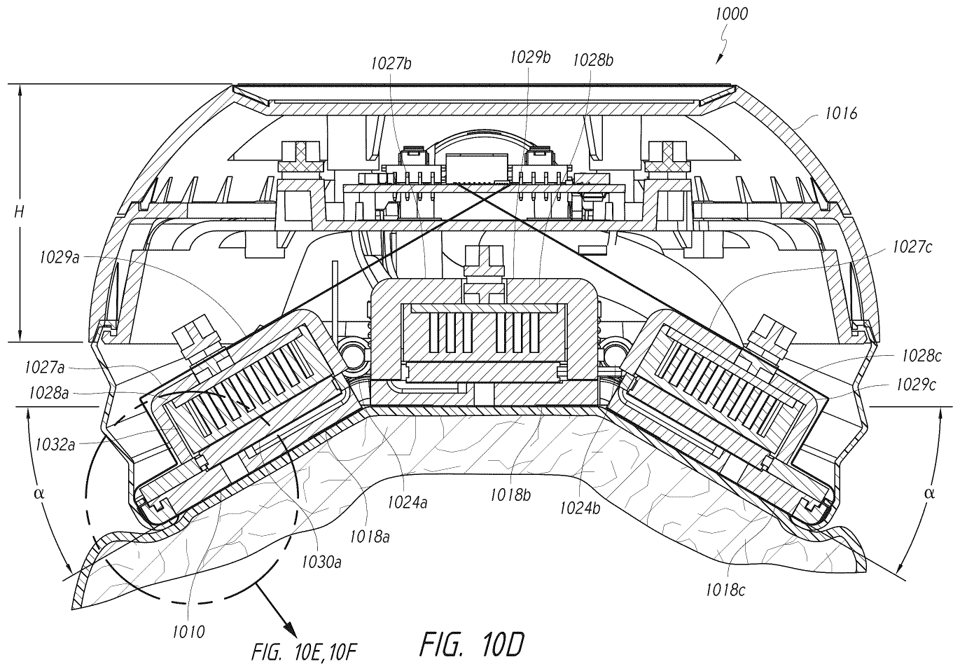

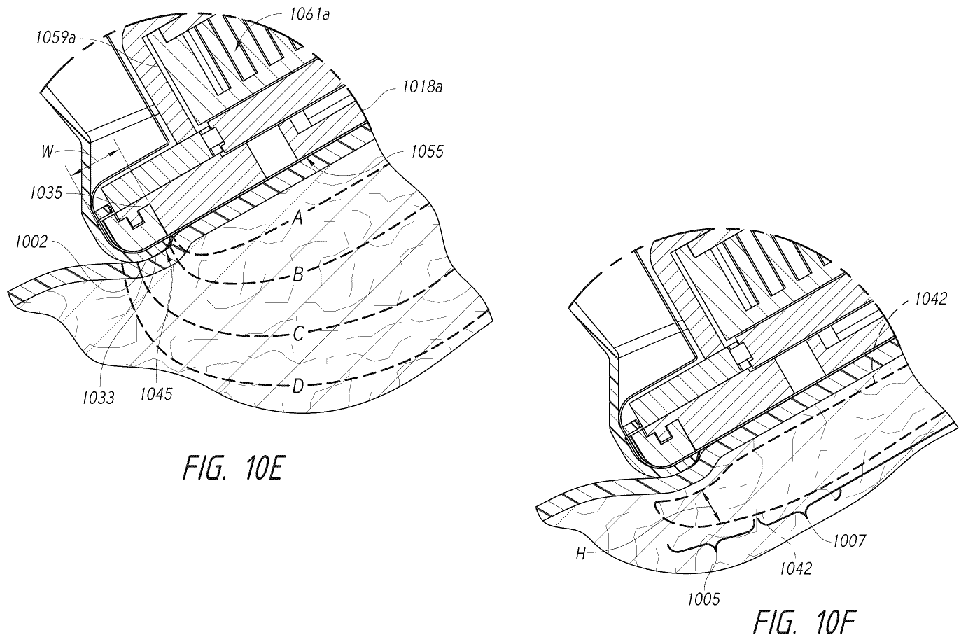

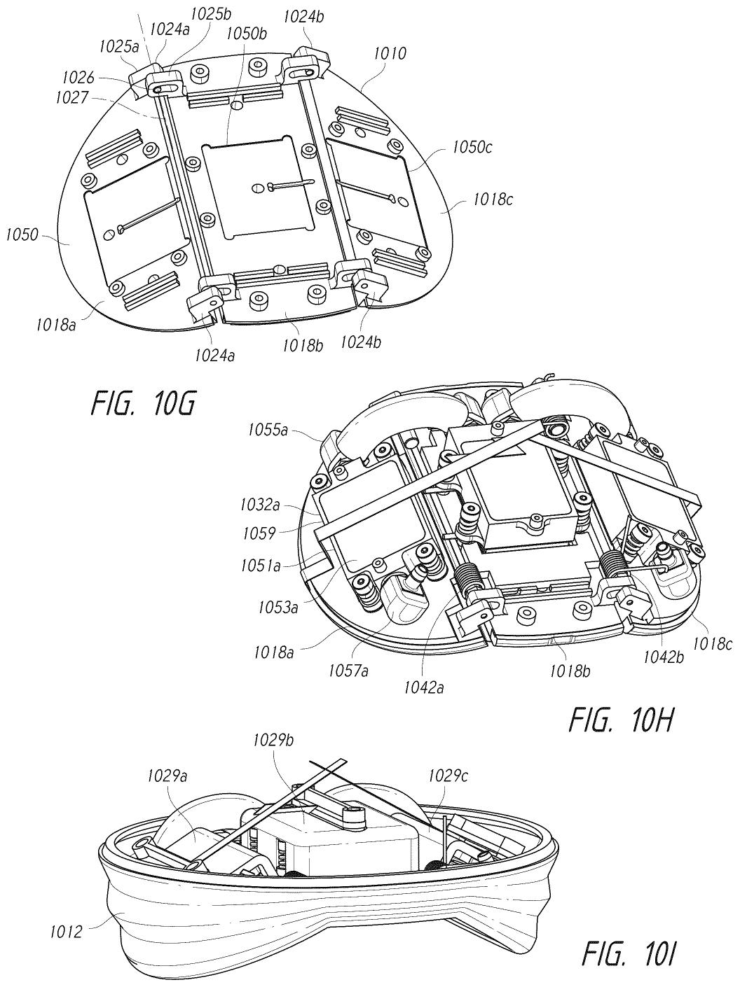

FIGS. 10A-10C illustrate a non-vacuum applicator 1000 (“applicator 1000”) configured in accordance with embodiments of the present technology. Referring now to FIG. 10A, the applicator 1000 includes an articulable panel assembly 1010 (“panel assembly 1010”), bellows 1012, and housing 1016. The panel assembly 1010 is configured to conform to highly contoured treatment sites to conductively cool a relatively large region of targeted tissue and is movable between configurations (e.g., from a first configuration, such as a planar configuration, to a second configuration, such as the illustrated angled configuration) to enable cooling of tissue without pulling or pinching tissue, thus enhancing comfort throughout therapy. The applicator 1000 can generate non-uniform temperature profiles to produce variable amounts of lipid-rich cell destruction to compensate for edge effects. In some embodiments, the non-uniform temperature profile can produce gradually decreasing tissue destruction at the periphery of the applicator 1000 to, for example, compensate for the edges of the applicator 1000 digging into the subject's skin, which would otherwise cause the formation of visible and permanent depressions.

Referring to FIG. 10B, the applicator 1000 has a temperature feathering feature 1002 defining a peripheral cooling zone 1005 (illustrated in dashed lines) that is warmer than an inner or interior cooling zone 1007 (illustrated in dashed line) defined by exposed thermally-conductive surfaces of the panel assembly 1010. The panel assembly 1010 has interconnected heat-exchanging elements 1018a-c that provide a generally continuous contact surface for conductively heating/cooling targeted tissue. The applicator 1000 can include optional thermistors 1019a for monitoring temperatures and thermistors 1019b for detecting adverse events, such as freeze events (e.g., skin freezes). The position, number, and capabilities of the thermistors, detectors, and other detection elements can be selected based on desired monitoring capabilities.

Referring now to FIG. 10C, the size of the applicator 1000 can be selected based on the treatment to performed. In some embodiments, the applicator 1000 has a length L (FIG. 10C) within a range from 6 inches to 7 inches (e.g., 6.5 inches (165 mm)), a width W (FIG. 10C) within a range from 6 inches to 6.2 inches (e.g., 6.2 inches (157 mm)), and a height H (FIG. 10D) within a range from 3 inches to 4 inches (e.g., 3.5 inches (89 mm)). The total weight of the applicator 1000 (excluding the connector 1017 of FIG. 10A-10C) can be within a range from 1.5 lbs to 2.5 lbs (e.g., 2 lbs).

An optional strap system can be used to minimize, reduce, or substantially eliminate movement of the applicator relative to the subject. The strap system can couple to a backside 1009 (FIG. 10C) of the applicator 1000 and hold the applicator 1000 to keep the cooling units (e.g., all or most of internal cooling units) in thermal contact with the subject. The applicator 1000 and compliant targeted tissue can cooperate to provide a high amount of thermal contact and reduce, limit, or substantially eliminate gaps between the subject and the treatment system that would impair heat transfer, including when treating non-pinchable regions, such as non-pinchable fat bulges (e.g., saddlebags), abdominal regions, flank regions, etc. Straps, retention devices, adhesive borders, and other features usable with the applicator 1000 and other applicators disclosed herein are described in U.S. patent application Ser. No. 14/662,181, which is incorporated by reference in its entirety.

FIG. 10D is a cross-sectional view of the applicator 1000 taken along line 10D-10D of FIG. 10B when the applicator 1000 is positioned on a subject. The panel assembly 1010 can include cooling assemblies 1027a-c (collectively “cooling assemblies 1027”). The cooling assemblies 1027a-c include the heat-exchanging elements 1018a-c (collectively “exchanging elements 1018”), thermal units 1028a-c (collectively “cooling units 1028”) coupled to the respective heat-exchanging element 1018, and anti-condensation housings 1029a-c (collectively “anti-condensation housings 1029”). The description of one of the cooling assemblies 1027 applies to the others, except as indicated otherwise.

The heat-exchanging element 1018a can include a plate, a covering, film, temperature sensors, and/or thermistors. The plate can be flat or shaped (e.g., curved) and can be made of metal or other conductive material (e.g., a rigid conductive material, a flexible conductive material, etc.). The covering can be a film, a sheet, a sleeve, or other component suitable for defining an interface surface. In one embodiment, the covering can be positioned between the plate and the subject's skin. In other embodiments, an exposed surface of the planar plate can define the exposed surface of the applicator 1000. In some embodiments, the heat-exchanging elements 1018 can have radii of curvature in one or more directions (e.g., a radius of curvature in one direction, a first radius of curvature in a first direction and a second radius of curvature in a second direction, etc.). In one embodiment, a rigid or flexible heat-exchanging element 1018 can have a radius of curvature in a direction generally parallel to the length or width of its exposed surface. Additionally, each heat-exchanging element 1018 can have the same configuration (e.g., curvature). In other embodiments, the heat-exchanging elements 1018 can have different configurations (e.g., shapes, curvatures, etc.). Applicators disclosed herein can have one of more flat heat-exchanging elements and one or more non-planar or shaped heat-exchanging elements. For example, the heat-exchanging elements 1018a, 1018c can be flat, and the heat-exchanging element 1018b can be non-planar (e.g., curved, partially spherical, partially elliptical, etc.). The shapes, dimensions, and properties (e.g., rigidity, thermal conductivity, etc.) of the heat-exchanging elements and other components of the applicator 1000 can be selected to achieve the desired interaction and heat transfer with the subject.

The thermal unit 1028a can include a thermoelectric device 1030a and a fluid-cooled device 1032a. The thermoelectric device 1030a can be coupled to and in thermal contact with the heat-exchanging element 1018a. The thermoelectric device 1030a can be a single thermoelectric cooling device or include multiple addressable thermoelectric cooling devices (e.g., two, three, or four thermoelectric cooling devices, such as Peltier devices). The thermoelectric device 1030a can include a greater or lesser number of thermoelectric elements with a variety of shapes (e.g., square, rectangular, etc.). The fluid-cooled device 1032a can exchange heat with the backside of the thermoelectric device 1030a to keep the thermoelectric device 1030a at or below a targeted temperature. The anti-condensation housings 1029a-c (e.g., foam insulation) can cover cooled components to inhibit or prevent condensation on the cooled surface from reaching electronic connections. For example, the anti-condensation housings 1029a-c can encapsulate the respective thermal units 1028 to prevent or inhibit water from reaching surrounding electrical components.

FIGS. 10E and 10F are detailed views of the applicator 1000 and tissue. Referring now to FIG. 10E, the feathering feature 1002 can include a lip 1033 and a low thermal conductivity border 1035 (“border 1035”). The border 1035 can be spaced apart from the thermal unit 1028a and is comprised of a material (e.g., silicon, plastic, rubber, steel, etc.) that is less thermally conductive than the material (e.g., copper, aluminum, metal alloy, etc.) of the heat-exchanging element 1018a. The feathering feature 1002 can be less thermally conductive than the at least one heat-exchanging element 1018a to keep cooling surface 1045 at least 1° C., 2° C., 3° C., 4° C., 5° C., or 6° C. warmer than a temperature of the cooling surface 1055 when cooling the targeted tissue to temperatures below 0° C., −3° C., −5° C., −10° C., or −12° C. The temperature differential can be sufficient to produce different amounts of damage/reduction in different regions of the treatment region. Accordingly, although the feathering feature 1002 and element 1018a can both cool and damage tissue, the feathering feature 1002 can limit the absorption of heat to limit damage and/or reduction of the underlying lipid-rich cells while the lipid-rich cells directly underlying the at least one heat-exchanging element 1018a are damaged and/or reduced to a greater extent.

The element 1018a can be made of thermally conductive materials that at room temperature have a thermal conductivity equal to or greater than about 50 W/(mK), 100 W/(mK), 200 W/(mK), 300 W/(mK), 350 W/(mK), and ranges encompassing such thermal conductivities. The border 1035 and/or lip 1033 can have a thermal conductivity equal or less than 50%, 40%, 30%, 20%, or 10% of the thermal conductivity of the heat-exchanging element 1018a. In some embodiments, the border 1035 and/or lip 1033 can have a thermal conductivity at room temperature equal to or less than about 0.2 W/(mk), 0.5 W/(mK), 1 W/(mK), 2 W/(mk), or other suitable thermal conductivities. During a cooling cycle, a temperature along a peripheral cooling surface 1045 of the border 1035 is higher than the temperature at the cooling surface 1055 of the element 1018a. For example, the cooling surface 1045 defining the cooling zone 1005 (FIGS. 10B and 10F) can be kept at least 1° C., 2° C., 3° C., 4° C., 5° C., 6° C., 7° C., 8° C., 9° C., or 10° C. higher than an adjacent region of the surface 1055 defining the zone 1004 (FIG. 10B and FIG. 10F). In some embodiments, a ratio of the thermal conductivity of the material of the border 1035 to the thermal conductivity of the material of the element 1018a can be equal to or less than 0.1, 0.2, 0.4, 0.5, 0.6, 0.7, or 0.8. The featuring feature 1002 can have a width W (FIG. 10E) equal to or less than about 5 mm, 10 mm, 20 mm, or other suitable width. In some embodiments, the width W is in a range of 5 mm to 8 mm and configured to provide a temperature gradient (e.g., in the radially outward direction relative to the center of the applicator 1000) of about 1° C., 2° C., 3° C., 4° C., 5° C., 6° C., 7° C., 8° C., 9° C., 10° C., or 20° C. across the surface 1045 in the outwardly direction (e.g., relative to the center of the applicator 1000).

The thermal characteristics of the applicator 1000 can be selected to achieve rates of cooling and rewarming of targeted tissue. For example, feathering feature 1002 can be configured to provide a defined number of Joules per unit of area or volume per unit of time can be extracted. In some embodiment, the number of Joules per unit area (e.g., Joules/inches squared) is equal or less than 40%, 30%, 20%, 10%, or 5% of the number of Joules per unit area of cooling provided by the heat-exchanging element 1018a.

FIG. 10E also shows isothermal curves for the temperatures that are reached at different depths due to the cooling. By way of example, it is possible to achieve temperatures in which isotherm A=−15° C. to −8° C., B=−5° C. to 5° C., and C=−2° C. to 10° C. In some procedures, the temperature at a peripheral cooling surface 1045 of the border 1035 is at least 1° C., 2° C., 3° C., 4° C., 5° C., 6° C., 7° C., 8° C., 9° C., or 10° C. greater than the temperature (e.g., an average temperature) along an adjacent portion or the entire primary cooling surface 1055. In one procedure, the isotherm A=−10° C., B=0° C., C=5° C., and D=10° C. The applicator can be controlled to generate different isotherms during a session.

FIG. 10F shows the treatment zones 1005, 1007 in targeted tissue 1042 (illustrated in dashed line) associated with the isotherms of FIG. 10E. The peripheral treatment zone 1005 is directly below the cooling surface 1045 (FIG. 10E) of the featuring feature 1002. As indicated in dashed line, the volume of affected tissue in the peripheral treatment zone 1005 decreases in the outward direction. For example, the height of the peripheral zone 1005 can gradually decrease in toward the periphery of the applicator 1000. The inner treatment zone 1007 of the targeted tissue 1042 can have a generally uniform height. Other temperature distributions can be produced by controlling operation of the applicator 1000.

FIG. 10G illustrates the panel assembly 1010 including heat-exchanging elements 1018a-c coupled together by the hinges 1024a, 1024b. Each hinge 1024a, 1024b can include brackets 1025a, 1025b (one set of brackets is identified) and a pin 1026 (one identified). Each pin 1026 defines an axis of rotation 1027 (one identified) about which the cooling unit heat-exchanging element 1018a rotates an angle of rotation a (one angle of rotation a is identified for heat-exchanging element 1018a of FIG. 10D) that can be equal to or less than about 10 degrees, 20 degrees, 30 degrees, 40 degrees, or other desired degrees of rotation. For example, angle of rotation a (FIG. 10D) can be about 25 degrees (e.g., 25 degrees±3 degrees), 30 degrees (e.g., 30 degrees±3 degrees), 35 degrees (e.g., 30 degrees±3 degrees) to provide rotation sufficient for conforming to outer thigh curvatures. The total cooling plate area of the panel assembly 1010 can be about 18 in², 19 in², 20 in², 21 in², 22 in², 23 in², or 23 in². For example, the cooling plate area (without or without the periphery cooling zone/lip) can about 19 in² (122 mm²), 20 in² (134 mm²), or the like.

FIG. 10H illustrates the internal components of the applicator 1000 with a spring assembly 1042a biasing the heat-exchanging element 1018a relative to the heat-exchanging element 1018b, and a spring assembly 1042b biasing the heat-exchanging element 1018c relative to the heat-exchanging elements 1018b. The spring assemblies 1042a, 1042b cooperate to position the heat-exchanging elements 1018 at predetermined bend angles relative to one another and can overcome the inherent stiffness of the bellows 1012 (FIGS. 10A and 10I). The spring assemblies 1042a, 1042b can provide sufficient biasing forces to pre-bend the applicator 1000 to prevent such over-tensioning of the straps, reduce or prevent lift off of the heat-exchanging elements 1018, or otherwise enhance performance.

Referring now to FIGS. 10E, 10G, and 10H, the heat-exchanging elements 1018a-c can include respectively recessed regions 1050a-c (FIG. 10G) for receiving cooling units. The fluid-cooled device 1032a (FIG. 10H) includes a fluid-cooled element 1051a, a cover 1053a, and inlet and outlet ports 1055a, 1057a. The fluid-cooled element 1051a can include a main body 1059b and a fluid chamber 1061a (FIG. 10E). The configuration and heating/cooling capabilities of the fluid-cooled device 1032a can be selected based on the thermal performance of other thermal components of the applicator.

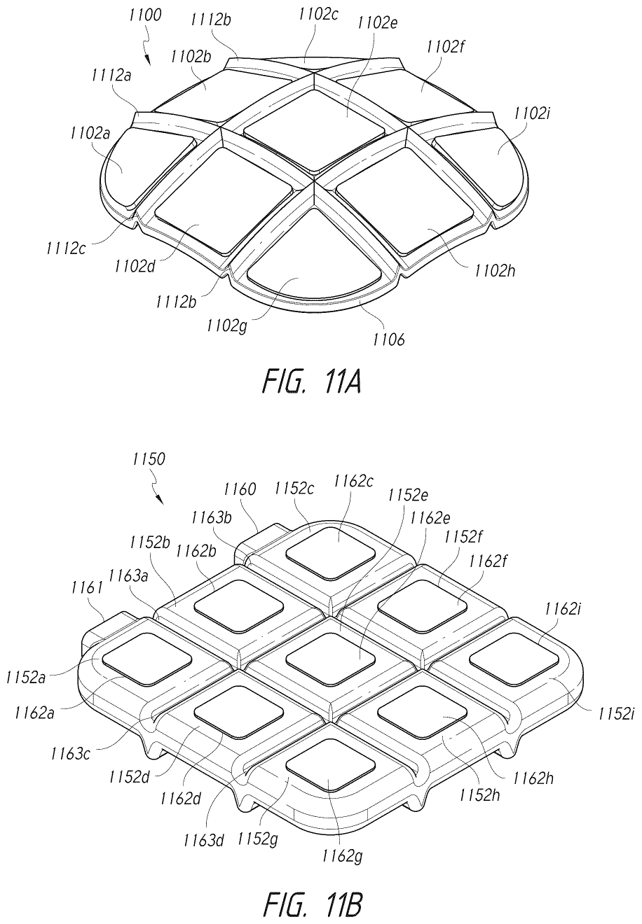

FIG. 11A illustrates a segmented or tiled thermal device 1100 (“device 1100”) suitable for use with a non-vacuum applicator in accordance with embodiments of the present technology. The device 1100 includes nine cooling units 1102a-i rotatable relative to one another. The cooling units 1102a-i can define a generally continuous cooling surface with a generally rounded square shape, rectangular shape, or circular shape. The cooling units 1102a-i can include one or more thermal elements (e.g., thermoelectric elements, fluid-cooled elements, temperature sensors, etc.). An articulating carrier 1106 can carry the cooling units 1102a-i and includes living hinges 1112a-d or other pivoting elements. The articulating carrier 1106 can be made, in whole or in part, of rubber, silicon, polymer, or other suitable flexible material.

FIG. 11B illustrates a tiled thermal device 1150 (“device 1150”) suitable for use with a non-vacuum applicator in accordance with embodiments of the present technology. The tiled thermal device 1150 includes nine cooling units 1152a-i rotatable relative to one another. The cooling units 1152a-i can define a generally continuous cooling surface with a generally rounded square shape, rectangular shape, or circular shape. The cooling units 1152a-i can include one or more thermal elements (e.g., fluid-cooled elements), cells, etc. The tiled thermal device 1150 can include an inlet 1160 and an outlet 1161. Coolant can flow through the inlet 1160, each of the cooling units 1152a-i, and exit the outlet 1163. The cooling units 1152a-i can include respective heat-exchanging plates 1162a-i (collectively “heat-exchanging plates 1162”) to facilitate heat transfer between the coolant and the exposed cooling surfaces of the heat-exchanging plates 1162.

The tiled thermal devices 1100, 1150 of FIGS. 11A-11B can be integrated with the applicator 1000 (FIGS. 10A-10F). In some embodiments, the tiled thermal devices 1100, 1150 are used together. Hinges 1112a-1112d (FIG. 11A) of the thermal device 1100 can fit in grooves 1163a-1163d (FIG. 11B) of the thermal device 1150. The configuration, shape, and functionality of the tiled thermal devices 1100, 1150 can be selected based on the area of tissue to be cooled, target temperature profiles, and other treatment parameters.

Retainer apparatuses, strap assemblies, and other components or features can be used with, or modified for use with, the applicators disclosed herein. The applicators disclosed herein can include additional features for providing a vacuum, energy (e.g., electrical energy, radiofrequency, ultrasound energy, thermal energy, etc.), and so forth. The treatment systems can include a pressurization device (e.g., a pump, a vacuum, etc.) that assists in providing contact between the applicator (such as via the interface layer or sleeve) and the patient's skin. In one embodiment, cooling units can include one or more vibrators (e.g., rotating unbalanced masses). In other embodiments, mechanical vibratory energy can be imparted to the patient's tissue by repeatedly applying and releasing a vacuum to the subject's tissue, for instance, to create a massage action during treatment. Further details regarding vacuum type devices and operation may be found in U.S. Patent Publication No. 2008/0287839. Exemplary components and features that can be incorporated into the applicators disclosed herein are described in, e.g., commonly assigned U.S. Pat. No. 7,854,754 and U.S. Patent Publication Nos. 2008/0077201, 2008/0077211, 2008/0287839, 2011/0238050 and 2011/0238051. The applicators disclosed herein may be cooled using only coolant, only thermoelectric elements, or other suitable features. In further embodiments, the treatment systems disclosed herein may also include a patient protection device incorporated into the applicators to prevent directed contact between the applicator and a patient's skin and thereby reduce the likelihood of cross-contamination between patients and/or minimize cleaning requirements for the applicator. The patient protection device may also include or incorporate various storage, computing, and communications devices, such as a radio frequency identification (RFID) component, allowing for example, use to be monitored and/or metered. Exemplary patient protection devices are described in commonly assigned U.S. Patent Publication No. 2008/0077201.

### H. Templates

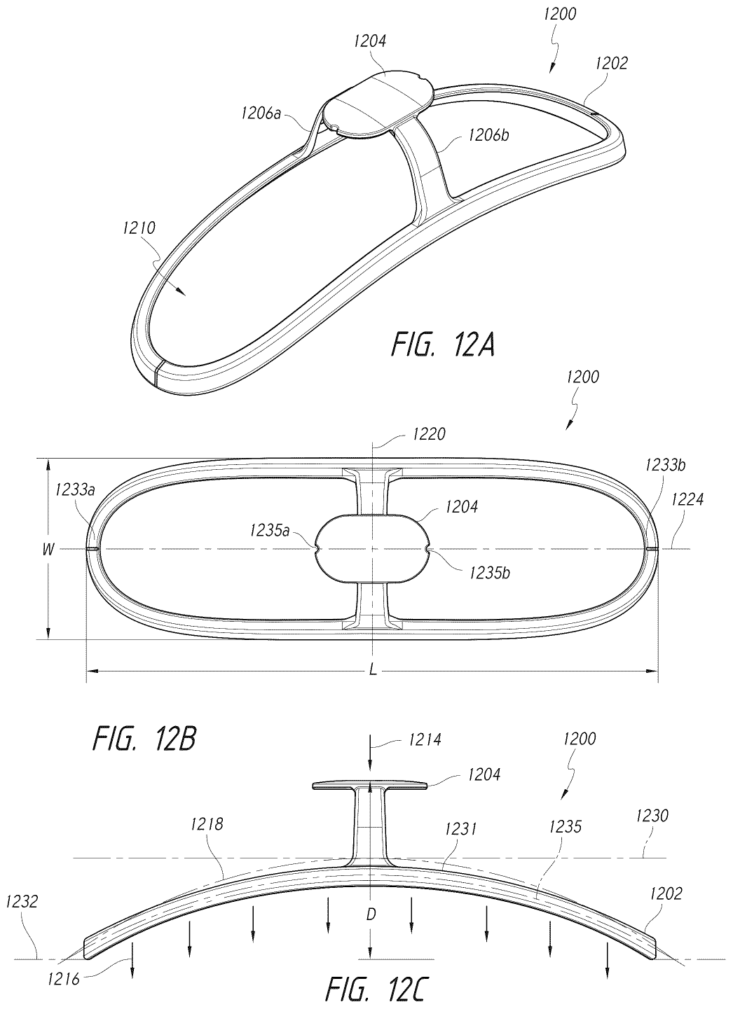

FIGS. 12A-18B illustrate applicator templates that be used to select an applicator suitable for a treatment site. The user can select and position an applicator template on the subject. If the applicator template fits the body part and surrounds the targeted tissue, the user can select a correspondingly dimensioned applicator. The user can also trace around the applicator template to provide a visual indicator for applicator placement. A treatment system can include an array of templates and corresponding applicators so that treatments can be performed on a wide range of subjects, different body parts, etc. Details of applicator templates are discussed below.

FIGS. 12A-12C illustrate an applicator template 1200 (“template 1200”) configured in accordance with embodiments of the present technology. Referring now to FIG. 12A (top isometric view), the template 1200 can include a treatment site frame 1202 (“frame 1202”), a handle 1204, and connectors 1206a, 1206b (collectively “connectors 1206”) extending between the frame 1202 and handle 1204. The frame 1202 can be substantially geometrically congruent to the tissue-engaging feature (e.g., mouth, sealing member, cup periphery, etc.) of an applicator. The handle 1204 extends outwardly from the connectors 1206 to provide an ergonomic structure that can be conveniently gripped by a user for manipulating the template 1200.

Referring now to FIG. 12B (top view), the handle 1204 can be positioned generally centrally relative to the frame 1202, as viewed from above. For example, the handle 1204 can be positioned along a midsagittal plane 1220 and/or a coronal plane 1224 of the frame 1202. The frame 1202 can include one or more alignment features 1233a, 1233b (e.g., notches, grooves, printed indicia, etc.) for facilitating positioning of the template 1200. Additionally or alternatively, the handle 1204 can have alignment features 1235a, 1235b. In some embodiments, the alignment features 1233a, 1233b are positioned on opposite sides of the frame 1202 and generally along the coronal plane 1224. The alignment features 1233a, 1233b and/or alignment features 1235a, 1235b can be used to draw lines (e.g., horizontal centerlines, vertical centerlines, etc.) drawn on the subject to center the template with respect to the treatment site.

Referring to FIG. 12C (side view), the handle 1204 and frame 1202 can be located on opposite sides of a transverse plane 1230 of the applicator 1200. When a user presses down on the handle 1204 (indicated by arrow 1214), the frame 1202 can apply a generally uniform pressure (indicated by arrows 1216) to the subject's tissue 1232 (illustrated in dashed line) to simulate how the tissue will respond when an applicator applies vacuum. Longitudinal sides 1231 of the frame 1202 can have curved longitudinal axis 1235 extending along a substantially circular path, an elliptical path, parabolic path, or other desired nonlinear or linear path. In some embodiments, the longitudinal axis 1235 has a curvature generally equal to the curvature (as viewed from the side) of the mouth or sealing member of the applicator. In other embodiments, the longitudinal axis 1235 can have a curvature selected based on the shape of the subject's body. When the frame 1202 is pressed against the subject's body, a portion 1218 of subject's tissue can bulge through an opening or window 1210 (FIG. 12A) indicating how the tissue will displace when pulled under vacuum.

In some embodiments, dimensions of the frame 1202 can correspond and be substantially equal to (e.g., ±5%) the dimensions of a cup. For example, the template 1200 can be configured to match the applicator of FIGS. 3A-3C. In some embodiments, a width W and length L of the frame 1202 (FIG. 12B) can match the corresponding width W₁ and length L₁ of the cup 306 (FIGS. 3B and 3C). For example, the ratio of the frame width W (FIG. 12B) to the cup width W₁ (FIG. 3B) can be about 0.9, about 0.95, about 1, about 1.05, or about 1.1, or within a range of such ratios (e.g., a range between 0.9 and 1.1). In some embodiments, the frame width W (FIG. 12B) and/or cup width W₁ (FIG. 3B) are within a range from 2 inches to 3 inches (e.g., 2.3 inches), the frame length L (FIG. 12B) and/or cup length L₁ (FIG. 3C) are within a range from 9 inches to 10 inches (e.g., 9.54 inches), and the frame depth D (FIG. 12C) and/or cup depth D₁ (FIG. 3C) are within a range from 2 inches to 3 inches (e.g., 2.6 inches). The cup opening (e.g., the opening defined by contoured sealing element 308 of FIG. 3C) and/or the area of the window 1210 (FIG. 12A) can be within a range from 20 square inches to 40 square inches (e.g., 33, 34, 35, or 36 square inches).

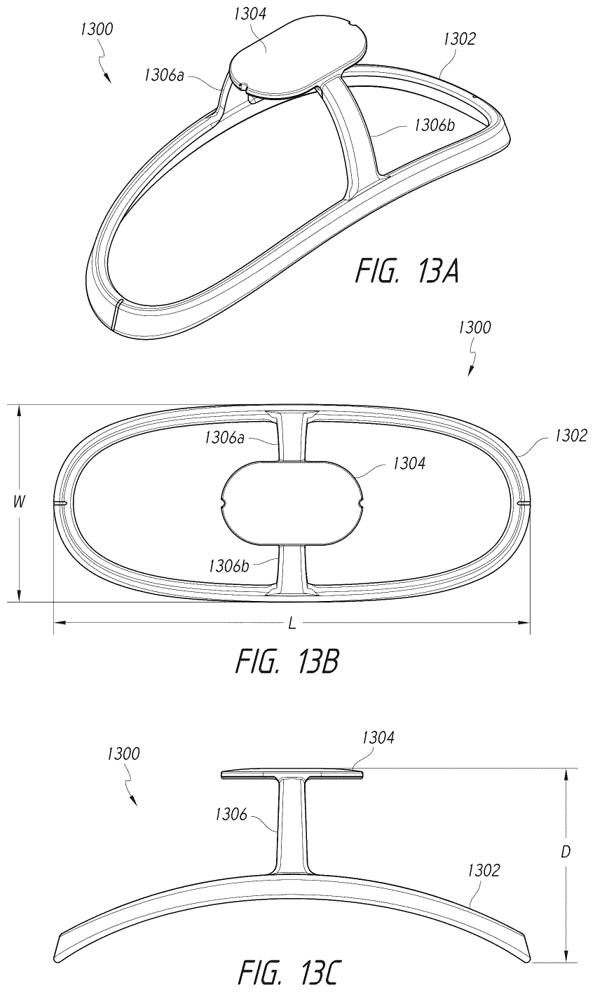

FIGS. 13A-13C illustrate an applicator template 1300 (“template 1300”) configured in accordance with embodiments of the present technology. Referring now to FIG. 13A (top isometric view), the template 1300 can include a treatment zone frame 1302 (“frame 1302”), a handle 1304, and connectors 1306a, 1306b (collectively, “connectors 1306”) extending between the frame 1302 and handle 1304. The frame 1302 can be substantially geometrically congruent to the mouth of an applicator (e.g., applicator 400 of FIGS. 4A-4C) for treating an intermediate size curved region. In some embodiments, a frame width W (FIG. 13B) and/or width W₂ of the cup 406 (FIG. 4A) are within a range from 2 inches to 3 inches (e.g., 2.3 inches), a frame length L (FIG. 13B) and/or the length L₂ of the cup 406 (FIG. 4B) are within a range from 5.5 inches to 6.5 inches (e.g., 6 inches), and a depth D (FIG. 13C) and/or the depth D₂ of the cup 406 (FIG. 4B) are within a range from 1.5 inches to 2.5 inches (e.g., 2 inches). The area of a window 1310 and/or the total treatment surface area (e.g., the area of surface 412 of FIG. 4B) can be within a range from 15 square inches to 25 square inches (e.g., 20 square inches).

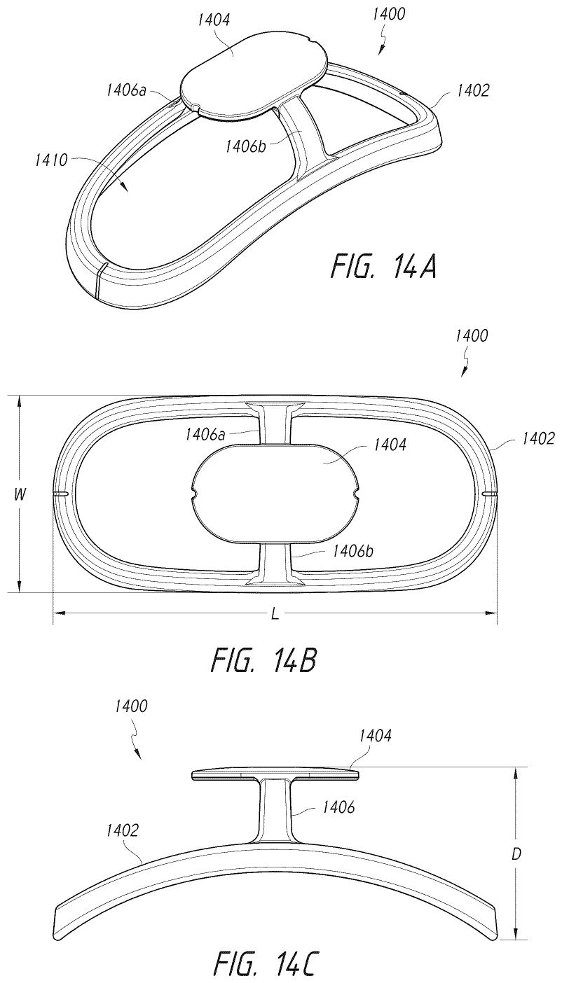

FIGS. 14A-14C illustrate an applicator template 1400 (“template 1400”) configured in accordance with embodiments of the present technology. Referring now to FIG. 14A (top isometric view), the template 1400 can include a treatment zone frame 1402 (“frame 1402”), a handle 1404, and connectors 1406a, 1406b (collectively, “connectors 1406”) extending between the frame 1402 and handle 1404. The frame 1402 can be substantially geometrically congruent to the mouth of an applicator (e.g., applicator 500 of FIGS. 5A-5C) for treating small sized curved regions. In some embodiments, a frame width W (FIG. 14B) and/or width W₃ of the cup 506 (FIG. 5A) are within a range from 1.5 inches to 2.5 inches (e.g., 1.9 inches), the frame length L (FIG. 14B) and/or length L₃ of the cup 506 (FIG. 5B) are within a range from 4.5 inches to 5.5 inches (e.g., 4.80 inches), and a frame depth D (FIG. 14C) and/or the depth D₃ of the cup 506 (FIG. 5B) are within a range from 1 inches to 2 inches (e.g., 1.51 inches). The area of the window 1410 and/or opening area (e.g., the area of the cup opening of FIG. 5B) can be within a range from 6 square inches to 18 square inches (e.g., 10-13 square inches).

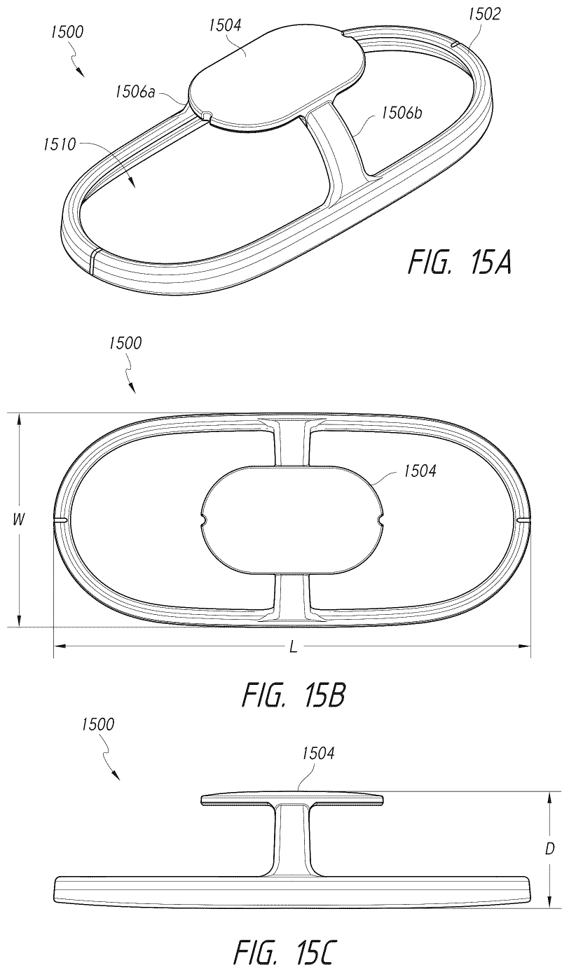

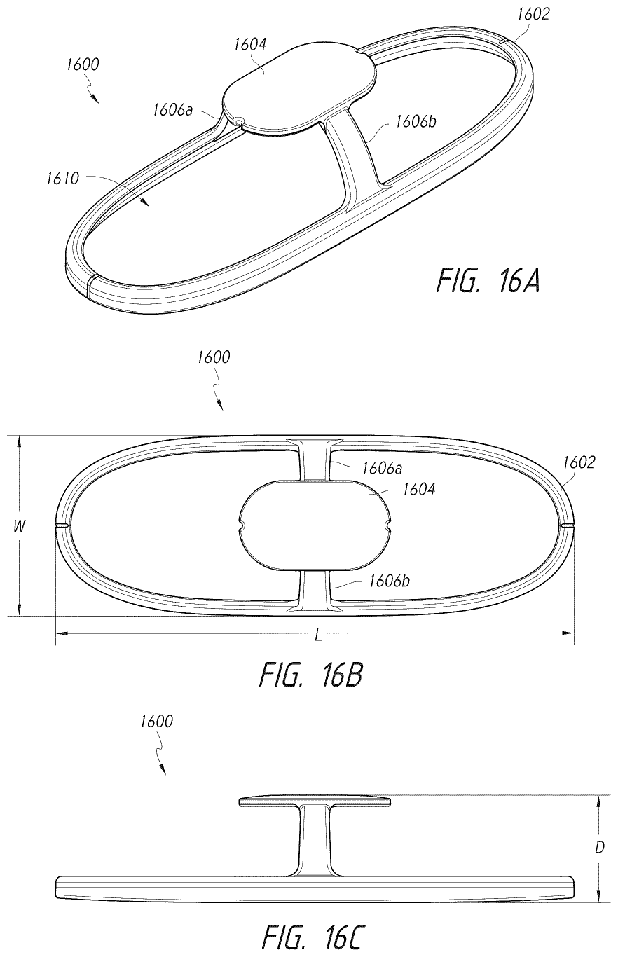

FIGS. 15A-15C illustrate an applicator template 1500 (“template 1500”) configured in accordance with embodiments of the present technology. Referring now to FIG. 15A (top isometric view), the template 1500 can include a treatment zone frame 1502 (“frame 1502”), a handle 1504, and connectors 1506a, 1506b (collectively, “connectors 1506”) extending between the frame 1502 and handle 1504. The frame 1502 can be substantially geometrically congruent to the mouth of an applicator (e.g., applicator 600 of FIGS. 6A-6C) for treating large flat regions. In some embodiments, the frame width W (FIG. 15B) and/or width W₄ of the cup 606 (FIG. 6A) is within a range from 1.5 inches to 2.5 inches (e.g., 2 inches), the frame length L (FIG. 16B) and/or length L₄ of the cup 606 (FIG. 6B) is within a range from 4.5 inches to 5.5 inches (e.g., 4.92 inches), and frame depth D (FIG. 15C) and/or the depth D₄ of the cup 606 (FIG. 7B) is within a range from 0.5 inches to 1.5 inches (e.g., 1 inch). The area of the window 1510 (FIG. 15A) and/or total treatment surface area (e.g., the area of surface 612) can be within a range from 8 square inches to 18 square inches (e.g., 12.9 square inches).

FIGS. 16A-16C illustrate an applicator template 1600 (“template 1600”) configured in accordance with embodiments of the present technology. Referring now to FIG. 16A (top isometric view), the template 1600 can include a treatment zone frame 1602 (“frame 1602”), a handle 1604, and connectors 1606a, 1606b (collectively, “connectors 1606”) extending between the frame 1602 and handle 1604. The frame 1602 can be substantially geometrically congruent to the mouth of an applicator (e.g., applicator 700 of FIGS. 7A-7C) for treating elongated flat regions. In some embodiments, for example, the frame width W (FIG. 16B) and/or width W₅ of the cup 706 (FIG. 7A) is within a range from 1.5 inches to 2.5 inches (e.g., 2 inches), the frame length L (FIG. 15B) and/or length L₅ of the cup 706 (FIG. 7B) is within a range from 6 inches to 7 inches (e.g., 6.5 inches), and the handle depth D (FIG. 15C) and/or the depth D₅ of the cup 706 (FIG. 7B) is within a range from 0.5 inch to 1.5 inches (e.g., 1.13 inches). The treatment window 1610 (FIG. 16A) and/or total treatment surface area (e.g., the area of surface 712) can be within a range from 10 square inches to 20 square inches (e.g., 15 square inches).

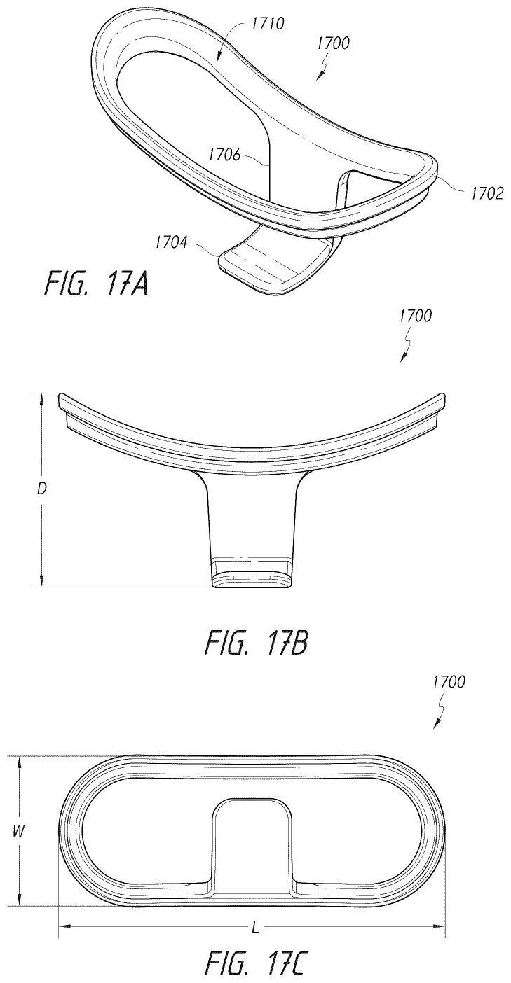

FIGS. 17A-17C illustrate an applicator template 1700 (“template 1700”) configured in accordance with embodiments of the present technology. Referring now to FIG. 17A (top isometric view), the template 1700 can include a treatment zone frame 1702 (“frame 1702”), a cantilevered handle 1704, and a connector 1706 extending between the frame 1702 and handle 1704. The frame 1702 can be substantially geometrically congruent to the mouth of an applicator (e.g., applicator 800 of FIGS. 8A-8C). The frame 1702 can be designed to be placed at a relatively small tissue area (e.g., a submental area). In some embodiments, for example, the frame width W (FIG. 17C) and/or width W₆ of the cup 806 (FIG. 8A) is within a range from 0.5 inches to 1.5 inches (e.g., 1.06 inches), the frame length L (FIG. 17C) and/or length L₆ of the cup 806 (FIG. 8A) is within a range from 2.5 inches to 3.5 inches (e.g., 3.15 inches), and depth D (FIG. 17B) and/or depth D₆ of the cup 806 (FIG. 8B) is within a range from 0.5 inches to 1.5 inches (e.g., 1.10 inches). The window area 1710 (FIG. 17A) and/or total treatment surface area (e.g., the area of surface 812) can be within a range from 2 square inches to 8 square inches (e.g., 5.4 square inches).

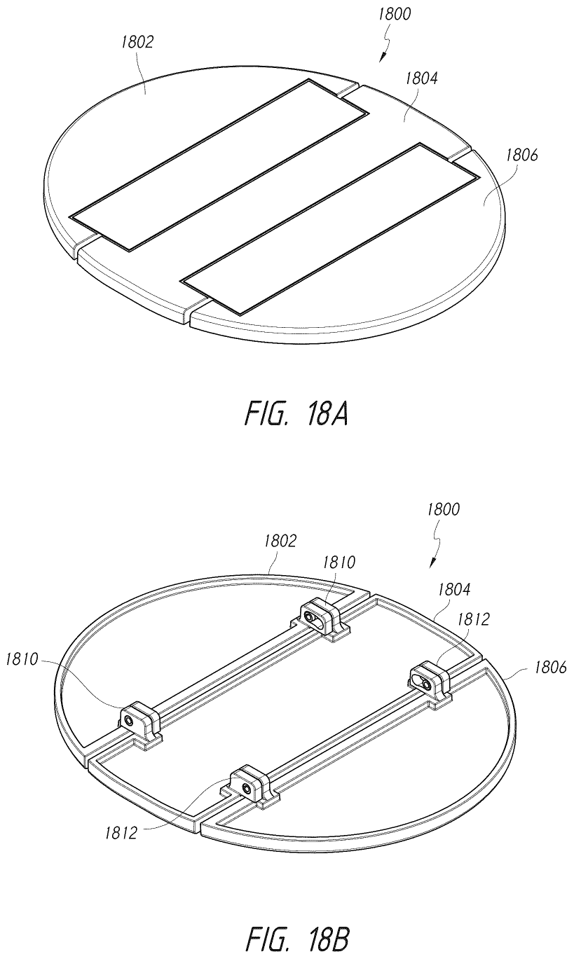

FIGS. 18A and 18B illustrate an applicator template 1800 (“template 1800”) configured in accordance with embodiments of the present technology. Referring now to FIG. 18A (top isometric view), the template 1800 can be substantially geometrically congruent to the applicator 1000 (FIGS. 10A-10I) and has three panels 1802, 1804, 1806 and hinges 1810, 1812 (FIG. 18B). The configuration of the template 1800 can generally match the configuration of the applicator 1000.

### I. Connector

FIGS. 19A-19K illustrate a connector 1900 configured in accordance with embodiments of the present technology. The connector 1900 can be used to connect an applicator (e.g., any of the applicators described herein with respect to FIGS. 1A-8B and 10A-10I) to a control unit (e.g., control unit 106 of FIG. 1A). In some embodiments, the connector 1900 is configured to couple various components of the applicator to corresponding components of the control unit. For example, the connector 1900 can be used to (i) fluidly couple a vacuum port of the applicator to a vacuum unit in the control unit (e.g., via vacuum line 125 of FIG. 1C), (ii) fluidly couple a fluid-cooled element of the applicator to a cooling unit in the control unit (e.g., via supply and return fluid lines 180a, 180b of FIG. 1C), and/or (iii) electrically couple a circuit board in the applicator to an applicator controller in the control unit (e.g., via electrical line 112 and/or control line 116 of FIG. 1C). As described in greater detail below, the connector 1900 can be releasably coupled to the applicator and/or control unit to allow different applicator types to be interchangeably used with a single control unit and vice-versa, and also to facilitate cleaning and storage.

Referring first to FIG. 19A (isometric view), the connector 1900 includes a distal end section 1902, a proximal end section 1904, and a flexible cable or umbilical 1906 extending between the distal and proximal end sections 1902, 1904. The cable 1906 can be an elongated, flexible structure with multiple lines or lumens running therethrough (e.g., vacuum lines, fluid lines, electrical lines, etc.—not shown). For example, the cable 1906 can provide be configured to: allow for coolant flow through the fluid lines at a rate from 0.8 LPM to 1.2 LPM (e.g., 1.0 LPM) with a maximum pump pressure of 90 psi; allow for a vacuum pressure of 8 inHg through the vacuum line; and/or provide electrical power through the electrical line at 15 VDC with a maximum current of 20 A. The cable 1906 can have a length L within a range from 40 inches to 80 inches (e.g., 49.7 inches, 61.6 inches, or 74.5 inches). The cable 1906 can have a minimum bend radius of 4 inches at a maximum bending force of 3 oz. The total weight of the connector 1900 can be within a range from 2 lbs to 3 lbs (e.g., 2.3 lbs, 2.57 lbs, or 2.86 lbs).

Referring next to FIGS. 19B and 19C, which show isometric and side views of the distal end section 1902, respectively, the distal end section 1902 can be configured to releasably couple to a distal end of an applicator. In the illustrated embodiments, the distal end section 1902 includes a distal interconnect receptacle 1920 and a join section 1922 connecting the distal interconnect receptacle 1920 to the cable 1906. The combined length L of the distal interconnect receptacle 1920 and join section 1922 can be within a range from 3.5 inches to 4.5 inches (e.g., 3.8 inches). The width W (or diameter) of the distal interconnect receptacle 1920 can be within a range from 1.5 inches to 2.5 inches (e.g., 2.2 inches).

As best seen in FIG. 19B, the distal interconnect receptacle 1920 can be an elongated hollow structure (e.g., a tube or cylinder) having a cavity 1924, a distal connector interface 1926 positioned within the cavity 1924, and one or more locking features 1927 formed in the inner wall of the distal interconnect receptacle 1920. The distal connector interface 1926 can include a base plate 1928 with various components for interfacing with a distal end of an applicator. For example, the distal connector interface 1926 can include a supply fluid line fitting 1930a, a return fluid line fitting 1930b, a vacuum line fitting 1932, and an electrical connector 1934. The supply and return fluid line fittings 1930a, 1930b can be fluidly coupled to supply and return fluid lines within the cable 1906. The vacuum fitting 1932 can be fluidly coupled to the vacuum line within the cable 1906. The electrical connector 1934 can be electrically coupled to electrical and/or control lines within the cable 1906. The locking features 1927 can be configured to releasably couple the distal interconnect receptacle 1920 to the distal end of the applicator, as discussed in greater detail below.

FIG. 19D is an isometric view of the distal end section 1902 of the connecter 1900 together with an interconnect assembly 1940 of an applicator (not shown); FIG. 19E is a front isometric view of the interconnect assembly 1940; and FIG. 19F is a back isometric view of the interconnect assembly 1940. Referring to FIGS. 19D-19F together, the interconnect assembly 1940 can be sized to fit at least partially within the cavity 1924 of the distal interconnect receptacle 1920. As best seen in FIGS. 19E and 19F, the interconnect assembly 1940 includes a proximal end portion 1942 shaped to be received within the cavity 1924, and a distal end portion 1944 configured to couple to the applicator. The interconnect assembly 1940 can also include a proximal sealing member 1945a configured to form a fluid seal between the interconnect assembly 1940 and the distal interconnect receptacle 1920, and a distal sealing member 1945b configured to form a fluid seal between the interconnect receptacle 1940 and a housing of the applicator (not shown). The proximal and distal sealing members 1945a, 1945b can be O-rings, gaskets, or any other structure that can provide a fluid-tight seal between components.

The proximal end portion 1942 of the interconnect assembly 1940 can include an applicator interface 1946 (FIG. 19E) that mates with the distal connector interface 1926 of the distal interconnect receptacle 1920. In the illustrated embodiment, the applicator interface 1946 includes a base plate 1948, a supply fluid line fitting 1950a, a return fluid line fitting 1950b, a vacuum line fitting 1952, and an electrical connector 1954. The supply fluid line fitting 1950a can connect to the supply fluid line fitting 1930a; the return fluid line fitting 1950b can connect to the return fluid line fitting 1930b; the vacuum line fitting 1952 can connect to the vacuum line fitting 1932; and the electrical connector 1954 can connect to the electrical connector 1934. Accordingly, by connecting the interconnect assembly 1940 to the distal interconnect receptacle 1920, the fluid lines, vacuum line, and electrical/control lines of the applicator can be connected to the fluid lines, vacuum line, and electrical/control lines of the connector 1900.

The supply fluid line fittings 1930a, 1950a, return fluid line fittings 1930b, 1950b, and vacuum line fittings 1932, 1952 (collectively, “distal interface fittings”) can be any connector suitable for fluidly coupling fluid lines, such as hose barb fittings. In some embodiments, some or all of the interface fittings are dripless fittings. The use of dripless fittings can allow the applicator to be water-tight, and can also minimize loss of coolant due to fitting losses, thus avoiding the need to periodically refill the treatment system with coolant (which may introduce issues with over- or under-filling). In some embodiments, the supply fluid line fittings 1930a, 1950a and the return fluid line fittings 1930b, 1950b, when coupled, have a maximum pressure drop of 7.0 psi per couple at a coolant flow rate of 1 LPM. When coupled, the supply fluid line fittings 1930a, 1950a and the return fluid line fittings 1930b, 1950b, can be configured to withstand fluid pressures of at least 90 psi, 110 psi, or 115 psi. The vacuum line fittings 1932, 1953, when coupled, can have a maximum pressure drop of 3.0 inHg at an air flow rate of 15 LPM, and can be configured to withstand vacuum pressures of at least −20 inHg.

The electrical connectors 1934, 1954 can be any connector suitable for electrically coupling electrical lines. For example, the electrical connector 1934 can be a socket with apertures and the electrical connector 1954 can be a plug with pins that fit into the apertures, or vice-versa. The electrical connectors 1934, 1954 can each have a plurality of pins for transmitting power, control signals, data, or other types of electrical signals. In the illustrated embodiment, the electrical connectors 1934, 1954 each have a fanned-out shape, which may be advantageous for reducing the sizes of the distal connector interface 1926 and the applicator interface 1946.

The proximal end portion 1942 of the interconnect assembly 1940 can further include one or more locking features 1957 (FIGS. 19E, 19F) configured to mate with the locking features 1927 of the distal interconnect receptacle 1920. For example, the locking features 1927, 1957 can be configured as a bayonet connector, with the locking features 1927 including one or more pins, protrusions, tabs, etc. and the locking features 1957 including one or more grooves, channels, recesses, etc., or vice-versa. In such embodiments, the interconnect assembly 1940 can be inserted into the distal interconnect receptacle 1920, then secured in place by rotating the interconnect assembly 1940 relative to the distal interconnect receptacle 1920 until the bayonet connector is locked. To release the interconnect assembly 1940 from the distal interconnect receptacle 1920, the interconnect assembly 1940 can be rotated relative to the distal interconnect receptacle 1920 in the opposite direction until the bayonet connector is unlocked. Optionally, the distal interconnect receptacle 1920 and the interconnect assembly 1940 can include visual indicators (e.g., arrows, coloring, etc.) that indicate the rotational directions for locking and unlocking the bayonet connector. In some embodiments, the maximum axial force to mate and/or unmate the interconnect assembly 1940 and the distal interconnect receptacle 1920 is less than or equal to 10 lb, 5 lb, or 1 lb. The maximum rotational torque to mate and/or unmate the interconnect assembly 1940 and the distal interconnect receptacle 1920 can be less than or equal to 15 lbf or 10 lbf.

FIGS. 19G and 19H are isometric and side views, respectively, of the proximal end section 1904 of the connector 1900. The proximal end section 1904 can be configured to releasably couple to a control unit (e.g., control unit 106 of FIG. 1A). In the illustrated embodiments, the proximal end section 1904 includes a proximal interconnect receptacle 1960 and a join or bend section 1962 connecting the proximal interconnect receptacle 1960 to the cable 1906. As shown in FIG. 19H, the longitudinal axis of the proximal interconnect receptacle 1960 can be offset from (e.g., orthogonal to) the longitudinal axis of the cable 1906 such that the join section 1962 has a bend of about 90 degrees. The combined length L₁ of the proximal interconnect receptacle 1960 and bend section 1962 as measured along the longitudinal axis of the proximal interconnect receptacle 1960 can be within a range from 5 inches to 6 inches (e.g., 5.5 inches). The length L₂ of the bend section 1962 as measured along the longitudinal axis of the cable 1906 can be within a range from 3.5 inches to 4.5 inches (e.g., 4 inches). The width W (or diameter) of the proximal interconnect receptacle 1960 can be within a range from 2.5 inches to 3.5 inches (e.g., 2.7 inches).

Referring again to FIG. 19G, the proximal interconnect receptacle 1960 can be an elongated hollow structure (e.g., a tube or cylinder) having a cavity 1964, a proximal connector interface 1966 positioned within the cavity 1964, and one or more locking features 1967 formed in the inner wall of the proximal interconnect receptacle 1960. The proximal connector interface 1966 can include a base plate 1968 with various components for interfacing with a control unit. For example, the proximal connector interface 1966 can include a supply fluid line fitting 1970a, a return fluid line fitting 1970b, a vacuum line fitting 1972, and an electrical connector 1974. The supply and return fluid line fittings 1970a, 1970b can be fluidly coupled to supply and return fluid lines within the cable 1906. The vacuum fitting 1972 can be fluidly coupled to the vacuum line within the cable 1906. The electrical connector 1974 can be electrically coupled to electrical and/or control lines within the cable 1906. The locking features 1967 can be configured to releasably couple the proximal interconnect receptacle 1960 to the control unit, as discussed in greater detail below.

FIGS. 191-19K are isometric, front, and side views, respectively, of an interconnect mount 1980 of a control unit (not shown). The interconnect mount 1980 can be sized to fit at least partially within the cavity 1964 of the proximal interconnect receptacle 1960. The interconnect mount 1980 can including a mounting plate 1982 and a body portion 1984 extending outwardly away from the surface of the mounting plate 1982. The mounting plate 1982 can have a generally flat shape with one or more apertures 1985 for securing the mounting plate 1982 to the control unit with fasteners (e.g., screws). For example, the mounting plate 1982 can be attached to a housing of the control unit (e.g., housing 124 of FIG. 1A).

The body portion 1984 can have an elongated shape (e.g., a cylindrical shape) that terminates in a console interface 1986. The console interface 1986 can be configured to mate with the proximal connector interface 1966 of the proximal interconnect receptacle 1960. As shown in FIGS. 191 and 19J, the console interface 1986 includes a base plate 1988, a supply fluid line fitting 1990a, a return fluid line fitting 1990b, a vacuum line fitting 1992, and an electrical connector 1994. Referring to FIGS. 19G-19K together, the supply fluid line fitting 1990a can connect to the supply fluid line fitting 1970a; the return fluid line fitting 1990b can connect to the return fluid line fitting 1970b; the vacuum line fitting 1992 can connect to the vacuum line fitting 1972; and the electrical connector 1994 can connect to the electrical connector 1974. Accordingly, by connecting the interconnect mount 1980 to the proximal interconnect receptacle 1960, the fluid lines, vacuum line, and electrical/control lines of the control unit can be connected to the fluid lines, vacuum line, and electrical/control lines of the connector 1900.

The supply fluid line fittings 1970a, 1990a, return fluid line fittings 1970b, 1990b, and vacuum line fittings 1972, 1992 (collectively, “proximal interface fittings”) can be identical or generally similar to the corresponding distal interface fittings discussed above. For example, some or all of the proximal interface fittings can be dripless fittings. Likewise, the electrical connectors 1974, 1994 can be identical or generally similar to the electrical connectors 1934, 1954 described above, except that the electrical connectors 1974, 1994 may have a generally circular shape rather than a fanned-out shape.

Referring again to FIGS. 191-19K, the body portion 1984 of the interconnect mount 1980 can further include one or more locking features 1997 configured to mate with the locking features 1967 of the proximal interconnect receptacle 1960 (FIG. 19G). For example, the locking features 1967, 1997 can be configured as a bayonet connector, with the locking features 1967 including one or more pins, protrusions, tabs, etc. and the locking features 1997 including one or more grooves, channels, recesses, etc., or vice-versa. In such embodiments, the proximal interconnect receptacle 1960 can be positioned around the body portion 1984 of the interconnect mount 1980, then secured in place by rotating the proximal interconnect receptacle 1960 relative to the interconnect mount 1980 until the bayonet connector is locked. To release the proximal interconnect receptacle 1960 from the interconnect mount 1980, the proximal interconnect receptacle 1960 can be rotated relative to the interconnect mount 1980 in the opposite direction until the bayonet connector is unlocked. Optionally, the proximal interconnect receptacle 1960 and the interconnect mount 1980 can include visual indicators (e.g., arrows, symbols, etc.) that indicate the rotational directions for locking and unlocking the bayonet connector). In some embodiments, the maximum axial force to mate and/or unmate the proximal interconnect receptacle 1960 and the interconnect mount 1980 is less than or equal to 10 lb, 5 lb, or 1 lb. The maximum rotational torque to mate and/or unmate the proximal interconnect receptacle 1960 and the interconnect mount 1980 can be less than or equal to 15 lbf or 10 lbf.

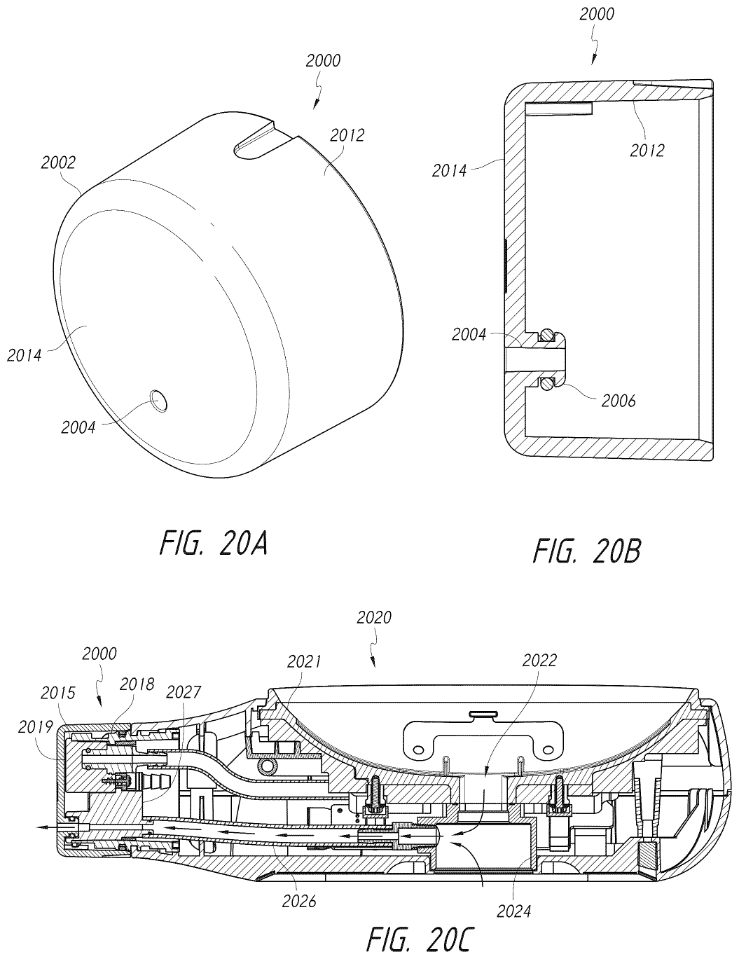

FIGS. 20A-20B illustrate a cleaning cap 2000 (“cap 2000”) configured in accordance with embodiments of the present technology. Referring now to FIG. 20A (isometric view) and FIG. 20B (side cross-sectional view), the cleaning cap 2000 can include a cap body 2002, a through-hole 2004, and a coupling feature 2006. The cap body 2002 can include a cylindrical sidewall 2012 and top wall 2014 and can be made, in whole or in part, of plastic, metal, or other material suitable for contacting a washing fluid. The cleaning cap 2000 is configured to cover and protect interconnect assemblies of applicators.

FIG. 20C is a cross-sectional view of the cap 2000 coupled to an applicator 2020 to allow a cleaning liquid to flow through components suitable for liquid contact. The cap 2000 is in fluid communication with the vacuum line 2026 to keep cleaning liquid from contacting other connectors (e.g., electrical connectors, coolant connectors, etc.) of the applicator 2200. A connector 2027 be coupled to the coupling feature 2006 and the vacuum line 2026. In other embodiments, the vacuum line 2026 is coupled directly to the coupling feature 2006. The top wall 2014 covers and obstructs other connections within the portion 2015 of a connector or interconnect assembly 2018 (“interconnect assembly 2018”). The top wall 2014 can include gaskets, sealing member, or other component for forming a seal (e.g., watertight seal, airtight seal, etc.) along the cap/connector interface 2019. Accordingly, the cap 2000 and interconnect assembly 2018 cooperate to seal internal connectors from the vacuum or air flow path along which fluid travels, as indicated by arrows.

To clean the applicator 2020, the cap 2000 can be coupled to the interconnect assembly 2018. If a gel trap is present, it can be removed from the applicator 2020. Liquid (e.g., water) can be sprayed against the cup 2021 to clean a cooling surface, air-egress features, etc. To remove substances from the vacuum flow path in the applicator 2020, the liquid can flow through a vacuum port 2022, manifold 2024, and vacuum line 2026. The liquid can be circulated along the flow path to remove gel and other contaminates that may have entered the internal air flow passageways while the cap 2000 prevents the water from contacting electrical components of the interconnect assembly 2018. The cap 2000 can be configured be used with any vacuum applicator disclosed herein.

The applicators disclosed herein may be waterproof according to at least the IPX1, IPX3, IPX4, IPX7, or other ingress Protection (IP) rating or standard for substance (e.g., water ingress) defined, for example, by ANSI/IEC 60529, IP test, or similar standard. For example, applicators (including housings, connectors, etc.) can be IPX1, IPX3, IPX4, or IPX7 compliant to allow users to wash the applicator using, for example, running water. The cap 2000 can protect the electrical components if the applicator 2020 is submerged in water. In some embodiments, the applicators can be waterproof when submerged in water at a depth of 2-9 feet for at least 1 minute, 2 minutes, 5 minutes, or 10 minutes. The connectors of the applicator 2020 can have an ingress protection IP54 rating (e.g., splash proof for electrical components).

FIGS. 21A and 21B illustrate a connector 2100 configured in accordance with embodiments of the present technology. The connector 2100 is shown together with the applicator 800 of FIGS. 8A and 8B. The connector 2100 includes a distal end section 2102, a proximal end section 2104, and a cable or umbilical 2106 extending between the distal and proximal end sections 2102, 2104. The distal end section 2102 can be permanently coupled to the applicator 800. The description of the connector 1900 of FIGS. 19A-19FD applies equally to the connector 2100. The applicator 800 and/or connector 2100 can have one or more features described in U.S. patent application Ser. No. 14/662,181 (U.S. Pat. No. 10,675,176) and U.S. Pat. No. 10,568,759, which are incorporated by reference in their entireties. For example, the applicator 800 can include one more cooling units, fluid lines, vacuum lines, or connections disclosed in U.S. Pat. No. 10,568,759. In some embodiments, the connector 2100 includes supply and return fluid lines 2120a, 2120b and electrical line 2124. The supply and return fluid lines 2120a, 2120b can be coupled to a supply and return fluid line fittings of the proximal end section 2104 and/or internal fittings of the applicator 800 (FIG. 8B). The connector 2100 can include one or more vacuum lines 2148 coupled to a vacuum fitting and an internal vacuum fitting 2146 (FIG. 8B) of the applicator 800.

The connection between the applicator 800 and connector 2100 can be waterproof according to at least IPX1, IPX3, IPX4, IPX7, or other ingress Protection (IP) rating or standard for substance (e.g., water ingress) defined, for example, by ANSI/IEC 60529, IP test, or similar standard. For example, the connection can be IPX1, IPX3, IPX4, or IPX7 compliant to allow users to wash the applicator 800 using, for example, running water. An internal distal end 2160 of a connector or hose 2106 can be adhered to applicator 800 to provide a watertight connection. One or more sealing members 2164 (e.g., O-rings, gaskets, etc.) can provide sealing between components at the connection. In some embodiments, a protective sleeve 2170 covers interfaces, joints, sealing members, etc. to further inhibit fluid ingress/egress. A proximal end 2180 of the hose 2106 can be adhered to a connector 2181 to provide a watertight connection. One or more sealing members 2184 (e.g., O-rings, gaskets, etc.) can provide sealing between components at the connection. In some embodiments, a protective sleeve 2190 covers interfaces, joints, sealing members, etc. to further inhibit fluid ingress/egress at interfaces. In some embodiments, the connections can be waterproof when submerged in water at a depth of 2-9 feet for at least 1 minute, 2 minutes, 5 minutes, or 10 minutes. This allows the applicator 800 and distal section of the connector 2106 to be submerged for cleaning.

### J. Control Unit

FIGS. 22A-22C illustrate a control unit 2200 configured in accordance with embodiments of the present technology. More specifically, FIG. 22A is a perspective view of the control unit 2200, FIG. 22B is a back view, and FIG. 22C is a side view. The control unit 2200 can include any of the features of the control unit 106 of FIG. 1A and/or the control unit 206 of FIG. 2A. For example, the control unit 2200 can include a housing 2202 with wheels 2204. The housing 2202 can include one or more interconnect mounts 2205 (FIG. 22B) for coupling to one or more applicators 2206 (e.g., a first applicator 2206a and a second applicator 2206b) via one or more respective connectors 2208 (e.g., a first connector 2208a and a second connector 2208b). In the illustrated embodiment, for example, the interconnect mounts 2205 are located in the back of the control unit 2200. The applicators 2206 can be any of the applicators described herein (e.g., with respect to any of FIGS. 1A-8B and 10A-10I), and the connectors 2208 can be any of the connectors described herein (e.g., with respect to any of FIGS. 1A-1C, 19A-191, and 21A-21B). The first applicator 2206a can be the same type of applicator as the second applicator 2206b, or can be a different type of applicator. Optionally, the control unit 2200 can include a bucket or receptacle 2210 (e.g., in the upper portion of the control unit 2200 (FIG. 22A)) for storing the applicators 2206 when not in use.

The control unit 2200 can include various functional components located within the housing 2202. For example, the control unit 2200 can include any of the systems and devices described herein, such as any of the components discussed above with respect to FIGS. 1A-2C (e.g., a cooling system, vacuum system(s), main controller, applicator controllers, computing device, power system, etc.). Some or all of the functional components can be operably coupled to the applicators 2206 via the interconnect mounts 2205 and connectors 2208, as previously described. The functional components can be accessed via a removable panel 2212 (e.g., in the back of the control unit 2200 (FIG. 22B)). The panel 2212 can include vents formed therein to allow heat generated by the functional components to escape.

For example, the control unit 2200 can house one or more applicator controllers (e.g., applicator controllers 224 of FIG. 2A—not shown in FIGS. 22A-22C) for monitoring and controlling the operation of the applicators 2206. As previously discussed, the electronics located onboard the applicators 2206 (e.g., circuit boards 210 of FIG. 2A) can have relatively limited functionality, e.g., to reduce the size, thermal footprint, weight, etc. of the applicators 2206. Instead, the applicator controllers within the control unit 2200 can receive and process data from the applicators 2206 (e.g., voltage data, current data, temperature data, etc.), and can transmit control and power signals to the applicators 2206. This approach can reduce costs by allowing a common set of applicator controllers to be used with different types of applicators 2206.

In some embodiments, the applicator controllers within the control unit 2200 are configured to power and control the operation of the thermoelectric elements within the applicators 2206. For example, the applicator controllers can be or include one or more TEC drivers configured for use with TECs. The TECs can be direct drive TECs, which may be more efficient than other types of TECs. The TEC drivers can measure the voltage and/or current to the TECs to determine the amount of power being delivered to the TECs, which may correlate to the amount of heat removed from the patient's tissue by the TECs. The voltage and/or current values can be used as feedback for controlling the amount of power delivered to the TECs, e.g., to improve treatment efficacy and safety.

Optionally, the TEC drivers can control the driving of each TEC individually, e.g., to independently control the amount of heat removed from the treatment zone corresponding to the TEC. For example, the TEC for each zone can be driven based on factors such as such as the measured temperature (e.g., of the patient's tissue at the particular zone and/or of the corresponding TEC), the power delivered to the corresponding TEC, the power delivered to other TECs, etc. In some embodiments, the driving algorithm for each zone uses a PID algorithm or loop. Different PID algorithms can be used for different applicators 2206. The inputs to the PID algorithm can include the power delivered to the TEC, the response to the measured temperature, and/or tuning parameters. The PID algorithm can assume that the amount of power commanded by the TEC driver is the same or similar to the actual amount of power delivered to the TEC. If the TEC driver detects that the commanded power is significantly different than the actual power delivered, this can indicate a problem in the system.

In some embodiments, the TEC drivers are configured to implement an anti-freeze process for reducing or avoiding freezing damage to the patient's skin surface. The tissue response to freezing can generate heat and cause the temperature of the skin surface to increase (e.g., from a target treatment temperature of −11° C. to a temperature within a range from −8° C. to −9° C. within 2-3 seconds). Accordingly, tissue freezing can be detected using temperature sensors (e.g., thermistors) within the applicator 2206 that are located adjacent or near the patient's skin (e.g., sensors 326 of FIGS. 3A-3I). If an increase in temperature indicative of tissue freezing is detected, the TEC drivers can initiate the anti-freeze process by switching the TECs from cooling mode to heating mode (e.g., by switching the polarity of the TECs). The anti-freeze process can involve heating the tissue to a temperature above freezing (e.g., to 5° C.) within a relatively short time frame (e.g., no more than 30 seconds after detection of skin freezing). In some embodiments, all of the treatment zones of the applicator 2206 are concurrently or sequentially switched from cooling to heating so that the entire treatment surface of the applicator 2206 is used to heat the tissue, e.g., to prevent propagation of freezing through tissue. The use of remote TEC drivers and direct drive TECs can allow for a faster anti-freeze response, thus improving the safety of the treatment procedure.

In some embodiments, the applicator controllers of the control unit 2200 are also configured to receive and process data from other electronic components of the applicators 2206, such as temperature data from one or more temperature sensors (e.g., thermistors). As previously described, each applicator 2206 can include thermistors (e.g., sensors 326, 333 of FIGS. 3C and 3F, or other temperature sensors) for monitoring the temperature of the patient's tissue and/or the temperature of the cold side of the TECs. The thermistors can be monitored to check for inaccuracies, malfunctions, or other issues with the treatment. In some embodiments, the temperature measurements are obtained using measurements of the thermistors by, e.g., applying a controlled voltage (e.g., bipolar measurements by applying bipolar voltage across the thermistors). The controlled voltage can originate in the control unit 2200. Temperature measurements can be obtained at any suitable sampling rate, such as 1 sample/sec. This approach can advantageously avoid or reduce problems associated with application of a constant voltage to the thermistors such as metal migration and tin whiskers.

The control unit 2200 can also include an input/output device 2214, such as a touchscreen display or monitor. The input/output device 2214 can be used by a physician or other operator to input data (e.g., commands, patient data, treatment data, etc.). For example, commands input by the physician can be converted into control signals for controlling operation of various functional components of the control unit 2200 (e.g., cooling system, vacuum system, applicator controllers, etc.). The input/output device 2214 can also be used to output information to the physician (e.g., treatment progress, sensor data, instructions, feedback, etc.). In some embodiments, sensor data and/or other data from the various functional components of the control unit 2200 can be converted into graphical, textual, audio, or other output that is shown to the physician via the input/output device 2214 so the physician can monitor treatment progress.

In some embodiments, the control unit 2200 can include other types of components for receiving input data, such as a reader or scanner 2221 (FIG. 22A).

The scanner 2221 can be integrated into the input/output device 2214 (e.g., integrated into the bottom of the touchscreen display), or can be separate from the input/output device 2214. The scanner can be an optical scanner configured to scan barcodes or other optical or image data. For example, the scanner can be used to scan a patient barcode (e.g., from an ID card or a mobile app) to verify the identity of the individual being treated and/or obtain demographic information. As another example, the scanner can be used to scan a physician barcode (e.g., from an ID card or mobile app) to verify the identity of the physician carrying out the treatment. Optionally, the scanner 2221 can be used to scan product barcodes or QR codes (e.g., from a product label) to track the use of gel pads or other consumables. Barcodes can be added to cards carried by personnel associated with the treatment (e.g., patients, physicians, other healthcare professionals) and/or printouts (e.g., treatment instructions, product sheets). In some embodiments, the barcodes are not used to enable treatment, but rather for proofing and verification purposes before the treatment commences.

Optionally, the control unit 2200 can be operably coupled to a notifier device (e.g., notifier device 103 of FIG. 1A) operated by the patient undergoing treatment. The notifier device can be a handheld device with a push button or other input element that allows the patient to send a notification to the provider (e.g., if the patient would like assistance from the system operator, attendant, physician). The notifier device can be operably coupled to the control unit 2200 via wireless communication (e., via a local area network, Bluetooth, WiFi, mobile network, etc.) or wired communication. When the control unit 2200 receives a notification, it can alert the provider via the input/device 2214 and/or via a mobile device carried by the physician. Optionally, the notifier device can be configured to wirelessly transmit the notification directly to the physician's mobile device, rather than indirectly via the control unit 2200.

Optionally, the control unit 2200 can include a reader 2216 (FIG. 22A). The reader 2216 can obtain information from machine readable cards (e.g., provider cards, patient cards, etc.), labels, barcodes, RFID tags, or other types of labels. The reader 2216 and/or scanner 2221 can include one or more card reader devices, scanners, optical sensors, cameras, light sources, bar code scanners, or other components for obtaining information. In some embodiments, the reader 2216 is a card reader device configured to read or obtain data from one or more magnetic strips, microchips, barcodes, or the like.

The information (e.g., provider information, consumable ID, patient information, etc.) from the reader 2216 and/or scanner 2221 can be sent to a controller (e.g., controller 114 of FIG. 1). The controller can evaluate a processing protocol based on the received information and can determine whether the processing protocol can be performed or modified. By way of example, if the controller determines that gel scanned by the scanner 2221 is not suitable for a planned procedure based on information from a card 2218 (e.g., provider or patient card), the system can notify the operator that another gel should be used. Alternatively, the controller can compensate for characteristics of the gel to enable the planned treatment to be performed. Additionally, the controller, reader 2216, and/or scanner 2221 can communicate with databases, such as an inventory tracking database to track applicators (e.g., to determine if an applicator is available for use), consumable inventory, or the like.

FIG. 22A illustrates the card 2218 readable by the reader 2216 in accordance with the present technology. The card 2218 can include microelectronics 2219 having memory, an input/output device, processor, or combinations thereof that provide, for example, computing, storage, and/or communications. In some embodiments, the microelectronics 2219 include one or more secure processors, smartcards, secure memory, or any combination thereof. Secure processors include smartcard devices that enable memory access through dynamic symmetric or asymmetric mutual authentication, data encryption, and other software-based or firmware-based security techniques. In some embodiments, the microelectronics 2219 is a chip configured for wireless communication.

The microelectronics 2219 can be used to, for example, meter treatment cycles (e.g., treatment sessions where each purchased cycle is a single treatment session). A user can purchase treatment credits for cycles, and the new credits can be added to card 2218. In some embodiments, the user can add new credits to the card as long as at least one unused credit remains on the card. The system will be able to perform a cycle when a non-zero number of credits remain and is prohibited when a zero number of credits remain prior to the intended use. The system deducts a credit each time that an applicator treatment is started (e.g., each applicator can be independently controlled to perform a separate cycle). For example, if two applicators start treatments (whether concurrently or sequentially), two credits would be deducted from the card 2218. A multi-use card 2218 can have cycles for multiple applicators, so a single available cycle allows simultaneous operation of multiple applicators. In some embodiments, the card 2218 can include different types of credits for different types of treatment cycles. For example, the card 2218 can include a credit that allows the system to perform two independent treatments using two applicators (e.g., simultaneously or sequentially). As another example, the card 2218 can include a credit that allows the system to perform a single treatment using a single applicator. In yet another example, the card 2218 can include a credit that allows any applicator compatible with the system to be used for treatment.

The amount of treatments for which the applicators and/or control unit 2200 can be used may be limited to an amount that is predetermined, e.g., pre-purchased by the system operator, patient, etc. Accordingly, when the number of cycles on the card 2218 has been reached, the system may communicate to the operator that it is necessary to obtain, e.g., purchase, additional cycles. Optionally, when no credits remain, the card 2218 can be automatically locked to prevent future treatments using the card 2218. The card 2218 can also terminate functions based on detection of unauthorized activity, such as tempering, unauthorized attempts to add credits/cycles, etc. The card 2218 may be replenished, for example, via the internet. In non-replenishable embodiments, the card 2218 can be discarded, and another disposable card can be purchased. The disposable card 2218 may include anti-tampering software or circuitry that prevents the addition of cycles/credits.

The microelectronics 2219 may also store, for example, patient profiles, profiles of treatment parameters, anti-tampering software, and/or limits. Examples of patient profiles may include patient vitals, health records, treatment history, etc. The microelectronics 2219 can store one or more profiles indicating applicators that can be used on the patient. Examples of treatment parameters may include targeted body part or tissue, duration of a treatment, target temperatures, temperature profiles, number of cycles or sessions in a treatment, heat extraction rate during a treatment, etc. A dual card can have cycles that enable multi-applicator treatments (e.g., treatments performed by two applicators at the same time). Examples of limits include, for example, limiting certain applicators, number of applicators, treatment limits (e.g., length of treatment, temperatures, etc.), limiting systems and/or operators in specific geographic regions to specific treatments, etc. Example territory limits can restrict which territories (e.g., based on geolocation data, stored territory data, etc.), counties, and/or systems the card can be used with, thereby limiting systems and/or operators in specific geographic regions to specific treatments. A set of territory codes written into the control unit, and the card can have one code to restrict which systems a card can be used with. The card security firmware and control unit security firmware can use information from the card 2218 to determine requirements to enable treatment on applicators. The card 2218 can download security firmware or other firmware. In some embodiments, the card 2218 includes platform compatibility data for restricting use of the card with specific systems whereas universal cards 2218 can be used across systems platforms, multi-applicator treatments (e.g., current use of applicators), and/or multiple territories. In some embodiments, the card 2218 includes one or more compatibility checks for checking applicator type, software (e.g., minimum software versions in the control unit), or the like.

The microelectronics 2219 may also store card type. The card type can be, for example, standard, solo, or multi-use or dual. A standard or solo card can store a single cycle for each applicator use. A cycle can be deducted each time a treatment is started. For treatment systems with multiple applicators, each applicator can be independently controlled and causes a deduction of a cycle for each use. A dual card deducts an available cycle for each treatment cycle and allows concurrent use of two applicators. The control unit can use the stored card type to determine GUIs, temperature controls, etc. The card security firmware and the control unit security firmware can use information from the card to determine requirements to enable treatment on one or both applicators.

The system can perform one or more authentication procedures, including one or more of the features and techniques disclosed in U.S. Pat. No. 8,523,927, the disclosure of which is incorporated herein by reference in its entirety. For example, the system may invoke the authenticate routine in response to obtaining information from the card 2218, when an applicator is connected to the system, selection of a program, etc. The routine can authenticate each component that is connected to the system. The routine may employ various mechanisms for authenticating components. As an example, one such mechanism is a concept known as trusted computing. When using the trusted computing concept, transactions between every component (e.g., card, applicator, umbilical, etc.) are secured, such as by using encryption, digital signatures, digital certificates, or other security techniques. When a component connects to the system, the component may be queried (e.g., challenged) for its authentication credentials, such as a digital certificate. The component could then provide its authentication credentials in response to the query. Another component that sent the query can then verify the authentication credentials, such as by verifying a one-way hash value, a private or public key, or other data that can be used to authenticate the component. The authentication credentials or authentication function can be stored in a secure processor memory, or in other secure memory (e.g., onboard memory of the applicator) that is associated with the component that is to be authenticated. In some embodiments, a querying component can provide a key to a queried component, and the queried component can respond by employing an authentication function, such as a one-way hash function, to produce a responsive key, such as a one-way hash value. The queried component can then respond to the query by providing the produced responsive key to the querying component. The two components can thus authenticate each other to establish a secure communications channel. Further communications between the authenticated components can transpire over the secure communications channel by using encrypted or unencrypted data. Various known encryption techniques can be employed.

### K. Kits and Treatment Methods

The various components described herein can be provided as a kit for treatment of a subject. A kit can include a plurality of applicators (e.g., two or more of the applicators described with respect to any of FIGS. 1A-8B and 10A-10I). At least some of the applicators can have different dimensions to treat differently-sized treatment sites. For example, two or more of the applicators can have different treatment area to weight ratios, treatment area to tissue-draw depth ratios, etc. Optionally, the kit can include a plurality of applicator templates (e.g., two or more of the applicator templates described with respect to any of FIGS. 12A-18B), each applicator template having dimensions corresponding to dimensions of a respective applicator. For example, each applicator template can have a frame that is approximately geometrically congruent to a lip of a respective applicator. The kit can also include one or more cleaning caps and at least one connector (e.g., as described above with respect to FIGS. 19A-19K) configured to operably couple a single applicator to a control unit of a treatment system. The kit can also include one or more gel traps (e.g., as described above with respect to FIGS. 9A-9G) and/or other accessories (e.g., gel pads, liners, straps, etc.).

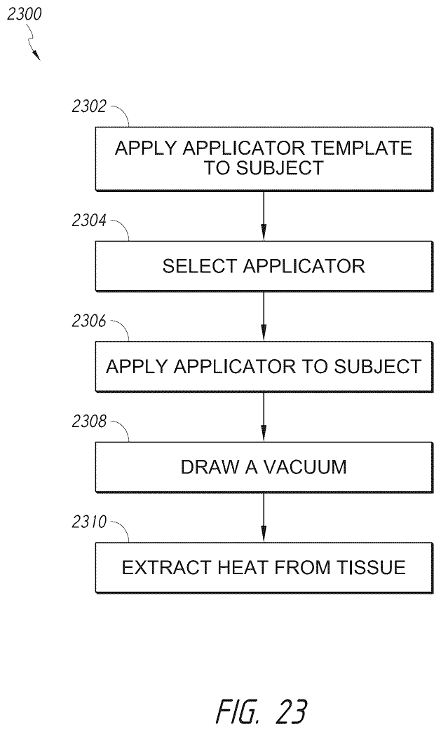

FIG. 23 is a flowchart of a method 2300 for treating a subject in accordance with embodiments of the present technology. Although certain features of the method 2300 are described with respect to the embodiments of FIGS. 1A-1C, it will be appreciated that the method 2300 can be performed using any of the systems and devices discussed with respect to FIGS. 1A-22C.

The method 2300 begins at step 2302 with applying an applicator template to the subject. The applicator template can be any of the embodiments described herein (e.g., with respect to any of FIGS. 12A-18B). The applicator template can be applied to a target treatment region on the subject's body, such as the submental region, abdomen, hips, legs, arms, face, neck, ankle region, or the like. In some embodiments, the physician or other user performing the treatment is provided with a kit of multiple applicator templates, each template having dimensions corresponding to a different applicator (e.g., any of the applicators of FIGS. 1A-8B and 10A-10I). The physician can select and position one of the applicator templates on the subject to visually assess whether the template fits the target region. Optionally, the physician can also trace the applicator template to provide a visual indicator for applicator placement.

At step 2304, if the applicator template fits the target region when applied to the subject, the applicator corresponding to the applicator template is selected for use in the treatment procedure. If, however, the applicator template does not fit the target region, the physician can select a different applicator template and check the fit of the new template against the subject's body. This process can be repeated multiple times until an appropriate applicator is selected. As previously discussed, the treatment systems herein can include a kit or array of multiple applicators having different dimensions, such that the physician can tailor the treatment to each patient's unique anatomy. In other embodiments, however, steps 2302 and/or 2304 are optional and may be omitted.

At step 2306, the selected applicator is applied to the subject's skin. The applicator can be any of the embodiments described herein (e.g., with respect to any of FIGS. 1A-8B and 10A-10I). In embodiments where the applicator is a vacuum applicator, step 2306 can further include engaging the skin with a sealing element of the applicator. For example, as discussed in connection with FIG. 1B, the sealing element 152 can be placed against the subject to form a seal suitable for maintaining a desired vacuum within the tissue-receiving cavity 158.

At step 2308, a vacuum is drawn to pull tissue into a tissue-receiving cavity of the applicator. The subject's skin can be drawn toward a temperature-controlled surface of a treatment cup of the applicator while air-egress features maintain airflow paths for removing air from the cavity. As discussed above, to draw the vacuum, a vacuum system (e.g., pressurization device 123 of FIG. 1A, vacuum system 218 of FIG. 2C) can operate to remove air from a tissue-receiving cavity of the applicator (e.g., tissue-receiving cavity 158 of FIG. 1B) to urge tissue into the applicator. The vacuum level can be selected to partially or completely fill the tissue-receiving cavity with tissue. If the vacuum level is too low, tissue will not be drawn adequately into the cavity. The vacuum level can be increased to reduce or eliminate gaps between the skin surface and a temperature-controlled surface (e.g., temperature-controlled surface 160) of the applicator. If the vacuum level is too high, undesirable discomfort to the patient and/or tissue damage could occur. The vacuum level can be selected to comfortably pull the tissue into contact with the desired area of the applicator, and the skin and underlying tissue can be pulled away from the subject's body which can assist in cooling underlying tissue by, e.g., lengthening the distance between targeted subcutaneous fat and the muscle tissue. As previously described, the vacuum system can be configured to rapidly achieve a target vacuum level (e.g., no more than 5 seconds, 4 seconds, 3 seconds, 2 seconds, or 1 second) with little or no undershoot or overshoot (e.g., no more than 20%, 15%, 10%, or 5% of the target vacuum level).

In some treatments, tissue can be drawn into the tissue-receiving cavity such that substantially all of the skin surface within the cavity overlies the temperature-controlled surface. For example, 90%, 95%, 99%, or more of the surface area of the skin located in the cavity can overlie the temperature-controlled surface. Optionally, the number and dimensions of the air-egress features can be increased or decreased to achieve desired thermal contact for a particular vacuum level. After a sufficient amount of tissue fills most or all of the cavity, the pressure level can be controlled to comfortably hold the tissue.

In other embodiments, step 2308 may be omitted, e.g., if the applicator is a non-vacuum applicator (e.g., as described with respect to FIGS. 10A-11B). In such embodiments, the treatment surface of the applicator can be designed to conform to the contours of the patient's skin without requiring application of a vacuum to draw the skin against the surface. For example, 90%, 95%, 99%, or more of the surface area of the skin can be placed in contact with the temperature-controlled surface without application of a vacuum.

At step 2310, the applicator can extract heat from the tissue. After the skin is in thermal contact with the temperature-controlled surface of the applicator, heat can be extracted from the subject's tissue to cool the tissue by an amount sufficient to be biologically effective in selectively damaging and/or reducing the subject's subcutaneous lipid-rich cells. As discussed above, the applicator can include a treatment cup (e.g., cup 156 of FIG. 1B) that is designed for rapid cooling and/or heating to, for example, reduce treatment times and/or produce generally flat temperature profiles over the temperature-controlled surface or a portion thereof. Because the subject's body heat can be rapidly conducted to the cup, the cooled skin can be kept at a generally flat temperature profile (e.g., +3° C. of a target temperature) even though regions of the skin, or underlying tissue, may experience different amounts of blood flow. Because non-lipid-rich cells usually can withstand colder temperatures better than lipid-rich cells, the subcutaneous lipid-rich cells can be injured selectively while maintaining the non-lipid-rich cells (e.g., non-lipid-rich cells in the dermis and epidermis). Accordingly, subcutaneous lipid-rich cells in a subcutaneous layer can be cooled an amount sufficient to be biologically effective in affecting (e.g., damaging and/or reducing) such lipid-rich cells without affecting non-target cells to the same or greater extent.

In contrast to invasive procedures in which coolant is injected directly into targeted tissue, the temperature-controlled surface can conductively cool tissue to produce a desired temperature in target tissue without bruising, pain, or other problems caused by injections and perfusion of injected fluid. For example, perfusion of injected fluid can affect the thermal characteristics of the treatment site and result in undesired temperature profiles. As such, the non-invasive conductive cooling provided by the applicator can be more accurate than invasive procedures that rely on injecting fluids. Targeted tissue can be cooled from about −20° C. to about 10° C., from about 0° C. to about 20° C., from about −15° C. to about 5° C., from about −5° C. to about 15° C., or from about −10° C. to about 0° C. In one embodiment, a liner can be kept at a temperature less than about 0° C. to extract heat from subcutaneous lipid-rich cells such that those cells are selectively reduced or damaged.

It may take a few days to a few weeks, or longer, for the adipocytes to break down and be absorbed. A significant decrease in fat thickness may occur gradually over 1-3 months following treatment. Additional treatments can be performed until a desired result is achieved. For example, one or more treatments can be performed to substantially reduce (e.g., visibly reduce) or eliminate targeted tissue. In such embodiments, the method 2300 can be repeated multiple times to achieve the desired treatment result.

Optionally, the method 2300 can include additional steps or processes not illustrated in FIG. 23. For example, the method 2300 can include positioning other elements, materials, components (e.g., gel pads, absorbents, etc.) between the skin and the applicator. U.S. Patent Publication No. 2007/0255362 and U.S. Patent Publication No. 2008/0077201 and U.S. application Ser. No. 14/610,807 disclose components, materials (e.g., coupling gels, cryoprotectants, compositions, etc.), and elements (e.g., coupling devices, liners/protective sleeves, absorbents, etc.) that can be placed between the skin and the applicator. Liners can be used and can include films, sheets, sleeves, or other components suitable for defining an interface surface to prevent direct contact between surfaces of the applicator and the subject's skin to reduce the likelihood of cross-contamination between patients, minimize cleaning requirements, etc. Exemplary protective liners can be sheets, sleeves, or other components constructed from latex, rubber, nylon, Kevlar®, or other substantially impermeable or semi-permeable material. For example, the liner can be a latex sheet coated with a pressure-sensitive adhesive. Further details regarding a patient protection device may be found in U.S. Patent Publication No. 2008/0077201. In some procedures, a liner or protective sleeve may be positioned between an absorbent and the applicator to shield the applicator and to provide a sanitary barrier that is, in some embodiments, inexpensive and thus disposable. After installing the liner assembly, gel traps, filters, valves, and other components can be installed to keep applied substances (e.g., coupling gels, cryoprotectants, etc.) from being sucked into and/or through the applicator. In some embodiments, the liner is configured to allow air to pass when drawing a vacuum and to restrict passage of a gel.

As another example, the method 2300 can include applying a cryoprotectant between the applicator and the skin. The cryoprotectant can be a freezing point temperature depressant that may additionally include a thickening agent, a pH buffer, a humectant, a surfactant, and/or other additives. The temperature depressant may include, for example, polypropylene glycol (PPG), polyethylene glycol (PEG), dimethyl sulfoxide (DMSO), or other suitable alcohol compounds. In a particular embodiment, a cryoprotectant may include about 30% polypropylene glycol, about 30% glycerin (a humectant), and about 40% ethanol. In another embodiment, a cryoprotectant may include about 40% propylene glycol, about 0.8% hydroxyethylcellulose (a thickening agent), and about 59.2% water. In a further embodiment, a cryoprotectant may include about 50% polypropylene glycol, about 40% glycerin, and about 10% ethanol. Other cryoprotectants or agents can also be used and can be carried by a cotton pad or other element. U.S. application Ser. No. 14/610,807 is incorporated by reference in its entirety and discloses various compositions that can be used as cryoprotectants.

In some embodiments, the method 2300 can include monitoring a temperature of the patient's tissue. It will be appreciated that while a region of the body has been cooled or heated to the target temperature, in actuality that region of the body may be close but not equal to the target temperature, e.g., because of the body's natural heating and cooling variations. Thus, although the applicator may attempt to heat or cool the target tissue to the target temperature or to provide a target heat flux, sensors may be used to measure a sufficiently close temperature or heat flux. If the target temperature or heat flux has not been reached, operation of the cooling unit can be adjusted to change the heat flux to maintain the target temperature or “set-point” selectively to affect targeted tissue. When the prescribed segment duration expires, the next treatment profile segment can be performed.

The sensors can be temperature sensors, such as thermistors, positioned to detect temperature changes associated with warm tissue being drawn into and/or located in the cup. A control unit (e.g., control unit 106 of FIG. 1A, control unit 206 of FIG. 2A) can interpret the detected temperature increase associated with skin contact and can monitor, for example, the depth of tissue draw, tissue, freezing, thawing, or the like. In some embodiments, sensors can be adjacent to the air-egress features and can measure heat flux and/or pressure (e.g., contact pressure) with the skin of the patient. In yet further embodiments, the sensors can be tissue impedance sensors, contact sensors, or other sensors used to determine the presence of tissue and/or whether tissue has been adequately drawn into the applicator so as to completely fill the cavity to achieve a suitable level of thermal contact, limit or reduce voids or gaps, and/or hold tissue while limiting or reducing, for example, pooling of blood, discomfort, and so forth.

Sensor feedback can be collected in real-time and used in concert with treatment administration to efficaciously target specific tissue. The sensor measurements can also indicate other changes or anomalies that can occur during treatment administration. For example, an increase in temperature detected by the sensors can indicate either a freezing event at the skin or movement of the applicator. An operator can inspect the subject's skin and/or applicator in response to a detected increase in temperature. Methods and systems for collection of feedback data and monitoring of temperature measurements are described in commonly assigned U.S. Pat. No. 8,285,390.

### L. Computing Environments

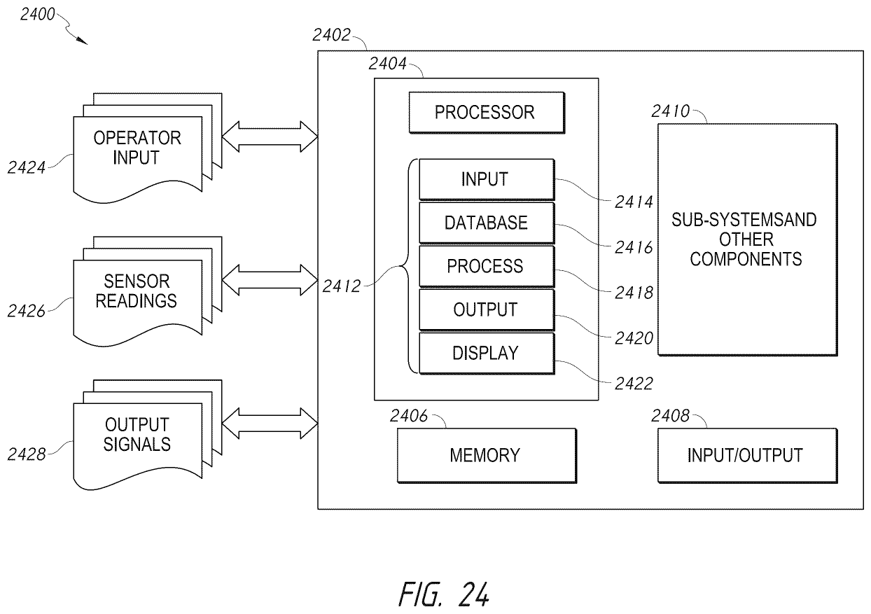

FIG. 24 is a schematic block diagram illustrating subcomponents of a controller 2400 in accordance with an embodiment of the disclosure. The controller can be part of a control unit (e.g., control unit 106 of FIG. 1A, control unit 206 of FIG. 2A) and/or can be incorporated into the applicators or other components disclosed herein. The controller 2400 can include a computing device 2402 having a processor 2404, a memory 2406, input/output devices 2408, and/or subsystems and other components 2410. The computing device 2402 can perform any of a wide variety of computing processing, storage, sensing, imaging, and/or other functions. Components of the computing device 2402 may be housed in a single unit or distributed over multiple, interconnected units (e.g., though a communications network). The components of the computing device 2402 can accordingly include local and/or remote memory storage devices and any of a wide variety of computer-readable media.

As illustrated in FIG. 24, the processor 2404 can include a plurality of functional modules 2412, such as software modules, for execution by the processor 2404. The various implementations of source code (i.e., in a conventional programming language) can be stored on a computer-readable storage medium or can be embodied on a transmission medium in a carrier wave. The modules 2412 of the processor can include an input module 2414, a database module 2416, a process module 2418, an output module 2420, and, optionally, a display module 2422.

In operation, the input module 2414 accepts an operator input 2424 via the one or more input devices, and communicates the accepted information or selections to other components for further processing. The database module 2416 organizes records, including patient records, treatment data sets, treatment profiles and operating records and other operator activities, and facilitates storing and retrieving of these records to and from a data storage device (e.g., internal memory 2406, an external database, etc.). Any type of database organization can be utilized, including a flat file system, hierarchical database, relational database, distributed database, etc.

In the illustrated example, the process module 2418 can generate control variables based on sensor readings 2426 from sensors and/or other data sources, and the output module 2420 can communicate operator input to external computing devices and control variables to the controller. The display module 2422 can be configured to convert and transmit processing parameters, sensor readings 2426, output signals 2428, input data, treatment profiles and prescribed operational parameters through one or more connected display devices, such as a display screen, touchscreen, printer, speaker system, etc.

In various embodiments, the processor 2404 can be a standard central processing unit or a secure processor. Secure processors can be special-purpose processors (e.g., reduced instruction set processor) that can withstand sophisticated attacks that attempt to extract data or programming logic. The secure processors may not have debugging pins that enable an external debugger to monitor the secure processor's execution or registers. In other embodiments, the system may employ a secure field programmable gate array, a smartcard, or other secure devices.

The memory 2406 can be standard memory, secure memory, or a combination of both memory types. By employing a secure processor and/or secure memory, the system can ensure that data and instructions are both highly secure and sensitive operations such as decryption are shielded from observation. In various embodiments, the memory 2406 can be flash memory, secure serial EEPROM, secure field programmable gate array, or secure application-specific integrated circuit. The memory 2406 can store instructions for causing the applicators to cool/heat tissue, pressurization devices to draw a vacuum, or other acts disclosed herein. Vacuum levels can be selected based on characteristics of the applicator, airflow features, and/or treatment site. In one embodiment, the memory 2406 stores instructions executable by the controller 2400 for the thermal device to sufficiently cool conductive cups disclosed herein such that vacuum applicators non-invasively cool the subcutaneous lipid-rich cells to a desired temperature, such as a temperature less than about 0° C. In some embodiments, the memory 2406 can contain liner installation or draw instructions for causing the liner to be drawn into the applicator, tissue draw instructions for causing the applicator to draw tissue into the applicator, treatment instructions for heating/cooling tissue, tissue release instructions for releasing tissue, and instructions for monitoring treatment. For example, the liner installation or draw instructions can be executed by the controller 2400 to command a vacuum system to suck the liner against a conductive surface of the conductive cup.

The input/output device 2408 can include, without limitation, a touchscreen, a keyboard, a mouse, a stylus, a push button, a switch, a potentiometer, a scanner, an audio component such as a microphone, or any other device suitable for accepting user input and can also include one or more video monitors, a medium reader, an audio device such as a speaker, any combination thereof, and any other device or devices suitable for providing user feedback. For example, if an applicator moves an undesirable amount during a treatment session, the input/output device 2408 can alert the subject and/or operator via an audible alarm. The input/output device 2408 can be a touch screen that functions as both an input device and an output device.

Optionally, the controller 2400 can include a control panel with visual indicator devices or controls (e.g., indicator lights, numerical displays, etc.) and/or audio indicator devices or controls. The control panel may be a component separate from the input/output device 2408, may be integrated with the applicators, may be partially integrated with one or more other devices, may be in another location, and so on. In alternative embodiments, the controller 2400 can be contained in, attached to, or integrated with the applicators. Further details with respect to components and/or operation of applicators, control modules (e.g., treatment units), and other components may be found in commonly-assigned U.S. Patent Publication No. 2008/0287839.

The controller 2400 can include any processor, Programmable Logic Controller, Distributed Control System, secure processor, and the like. A secure processor can be implemented as an integrated circuit with access-controlled physical interfaces; tamper resistant containment; means of detecting and responding to physical tampering; secure storage; and shielded execution of computer-executable instructions. Some secure processors also provide cryptographic accelerator circuitry. Suitable computing environments and other computing devices and user interfaces are described in commonly assigned U.S. Pat. No. 8,275,442, entitled “TREATMENT PLANNING SYSTEMS AND METHODS FOR BODY CONTOURING APPLICATIONS,” which is incorporated herein in its entirety by reference.

### M. Conclusion

The treatment systems, applicators, and methods of treatment can be used reduce adipose tissue or treat subcutaneous tissue, acne, hyperhidrosis, wrinkles, structures (e.g., structures in the epidermis, dermis, subcutaneous fat, muscle, nerve tissue, etc.), and so on. Systems, components, and techniques for reducing subcutaneous adipose tissue are disclosed in U.S. Pat. No. 7,367,341 titled “METHODS AND DEVICES FOR SELECTIVE DISRUPTION OF FATTY TISSUE BY CONTROLLED COOLING” to Anderson et al., U.S. Patent Publication No. US 2005/0251120 titled “METHODS AND DEVICES FOR DETECTION AND CONTROL OF SELECTIVE DISRUPTION OF FATTY TISSUE BY CONTROLLED COOLING” to Anderson et al., and U.S. Patent Publication No. 2007/0255362 titled “CRYOPROTECTANT FOR USE WITH A TREATMENT DEVICE FOR IMPROVED COOLING OF SUBCUTANEOUS LIPID-RICH CELLS,” the disclosures of which are incorporated herein by reference in their entireties. Vacuum applicators can stretch, stress, and/or mechanically alter skin to increase damage and fibrosis in the skin, affect glands, control freeze events (including initiating freeze events), etc. Methods for cooling tissue and related devices and systems in accordance with embodiments of the present invention can at least partially address one or more problems associated with conventional technologies as discussed above and/or other problems whether or not such problems are stated herein.

Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. Use of the word “or” in reference to a list of two or more items covers all of the following interpretations of the word: any of the items in the list, all of the items in the list, and any combination of the items in the list. Furthermore, the phrase “at least one of A, B, and C, etc.” is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.).

Reference throughout this specification to relative terms such as, for example, “generally,” “approximately,” and “about” are used herein to mean the stated value plus or minus 10%.

Any patents, applications and other references, including any that may be listed in accompanying filing papers, are incorporated herein by reference. Aspects of the described technology can be modified, if necessary, to employ the systems, functions, and concepts of the various references described above to provide yet further embodiments. These and other changes can be made in light of the above Detailed Description. While the above description details certain embodiments and describes the best mode contemplated, no matter how detailed, various changes can be made. Implementation details may vary considerably, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated.

1.-106. (canceled)

107. An applicator for selectively affecting a subject's subcutaneous tissue, comprising:
a housing;
a treatment cup mounted in the housing, wherein the treatment cup defines a tissue-receiving cavity and includes a temperature-controlled surface;
at least one thermal device coupled to the treatment cup and configured to cool the temperature-controlled surface; and
at least one vacuum port coupled to the treatment cup and configured to provide a vacuum to draw the subject's tissue into the tissue-receiving cavity and against the temperature-controlled surface to selectively damage and/or reduce the subject's subcutaneous tissue,
wherein the subject's tissue drawn into the tissue-receiving cavity comprises a skin surface area, and wherein at least 90% of the skin surface area contacts the temperature-controlled surface.
